SEQUENCE LISTING (94 sequences)

SEQ ID NO: 1 - DNA, 20 bp, Artificial Sequence
  Description of Artificial Sequence Synthetic oligonucleotide
ggcggcggcggcggcggcgg

SEQ ID NO: 2 - DNA, 20 bp, Artificial Sequence
  Description of Artificial Sequence Synthetic oligonucleotide
gcggcggcggcggcggcggc

SEQ ID NO: 3 - DNA, 20 bp, Artificial Sequence
  Description of Artificial Sequence Synthetic oligonucleotide
cggcggcggcggcggcggcg

SEQ ID NO: 4 - DNA, 20 bp, Artificial Sequence
  Description of Artificial Sequence Synthetic oligonucleotide
cgccgccgccgccgccgccg

SEQ ID NO: 5 - DNA, 20 bp, Artificial Sequence
  Description of Artificial Sequence Synthetic oligonucleotide
gccgccgccgccgccgccgc

SEQ ID NO: 6 - DNA, 20 bp, Artificial Sequence
  Description of Artificial Sequence Synthetic oligonucleotide
ccgccgccgccgccgccgcc

SEQ ID NO: 7 - DNA, 19 bp, Artificial Sequence
  Description of Artificial Sequence Synthetic oligonucleotide
ggggcgtgcggcagcgcgg

SEQ ID NO: 8 - DNA, 20 bp, Artificial Sequence
  Description of Artificial Sequence Synthetic oligonucleotide
ggcgtgcggcagcgcggcgg

SEQ ID NO: 9 - DNA, 20 bp, Artificial Sequence
  Description of Artificial Sequence Synthetic oligonucleotide
gtgcggcagcgcggcggcgg

SEQ ID NO: 10 - DNA, 20 bp, Artificial Sequence
  Description of Artificial Sequence Synthetic oligonucleotide
cgccgctgccagggggcgtg

SEQ ID NO: 11 - DNA, 20 bp, Artificial Sequence
  Description of Artificial Sequence Synthetic oligonucleotide
ggcactgcggctggaggtgg

SEQ ID NO: 12 - DNA, 20 bp, Artificial Sequence
  Description of Artificial Sequence Synthetic oligonucleotide
agcagcagcagcagcagcag

SEQ ID NO: 13 - DNA, 20 bp, Artificial Sequence
  Description of Artificial Sequence Synthetic oligonucleotide
gcagcagcagcagcagcagc

SEQ ID NO: 14 - DNA, 20 bp, Artificial Sequence
  Description of Artificial Sequence Synthetic oligonucleotide
cagcagcagcagcagcagca

SEQ ID NO: 15 - DNA, 20 bp, Artificial Sequence
  Description of Artificial Sequence Synthetic oligonucleotide
tgctgctgctgctgctgctg

SEQ ID NO: 16 - DNA, 20 bp, Artificial Sequence
  Description of Artificial Sequence Synthetic oligonucleotide
gctgctgctgctgctgctgc

SEQ ID NO: 17 - DNA, 20 bp, Artificial Sequence
  Description of Artificial Sequence Synthetic oligonucleotide
ctgctgctgctgctgctgct

SEQ ID NO: 18 - DNA, 20 bp, Artificial Sequence
  Description of Artificial Sequence Synthetic oligonucleotide
aagaagaagaagaagaagaa

SEQ ID NO: 19 - DNA, 20 bp, Artificial Sequence
  Description of Artificial Sequence Synthetic oligonucleotide
gaagaagaagaagaagaaga

SEQ ID NO: 20 - DNA, 20 bp, Artificial Sequence
  Description of Artificial Sequence Synthetic oligonucleotide
agaagaagaagaagaagaag

SEQ ID NO: 21 - DNA, 20 bp, Artificial Sequence
  Description of Artificial Sequence Synthetic oligonucleotide
ttcttcttcttcttcttctt

SEQ ID NO: 22 - DNA, 20 bp, Artificial Sequence
  Description of Artificial Sequence Synthetic oligonucleotide
tcttcttcttcttcttcttc

SEQ ID NO: 23 - DNA, 20 bp, Artificial Sequence
  Description of Artificial Sequence Synthetic oligonucleotide
cttcttcttcttcttcttct

SEQ ID NO: 24 - DNA, 20 bp, Artificial Sequence
  Description of Artificial Sequence Synthetic oligonucleotide
cccggccccggccccggccc

SEQ ID NO: 25 - DNA, 20 bp, Artificial Sequence
  Description of Artificial Sequence Synthetic oligonucleotide
ccggccccggccccggcccc

SEQ ID NO: 26 - DNA, 20 bp, Artificial Sequence
  Description of Artificial Sequence Synthetic oligonucleotide
cggccccggccccggccccg

SEQ ID NO: 27 - DNA, 20 bp, Artificial Sequence
  Description of Artificial Sequence Synthetic oligonucleotide
ggccccggccccggccccgg

SEQ ID NO: 28 - DNA, 20 bp, Artificial Sequence
  Description of Artificial Sequence Synthetic oligonucleotide
gccccggccccggccccggc

SEQ ID NO: 29 - DNA, 20 bp, Artificial Sequence
  Description of Artificial Sequence Synthetic oligonucleotide
ccccggccccggccccggcc

SEQ ID NO: 30 - DNA, 20 bp, Artificial Sequence
  Description of Artificial Sequence Synthetic oligonucleotide
gggccggggccggggccggg

SEQ ID NO: 31 - DNA, 20 bp, Artificial Sequence
  Description of Artificial Sequence Synthetic oligonucleotide
ggccggggccggggccgggg

SEQ ID NO: 32 - DNA, 20 bp, Artificial Sequence
  Description of Artificial Sequence Synthetic oligonucleotide
gccggggccggggccggggc

SEQ ID NO: 33 - DNA, 20 bp, Artificial Sequence
  Description of Artificial Sequence Synthetic oligonucleotide
ccggggccggggccggggcc

SEQ ID NO: 34 - DNA, 20 bp, Artificial Sequence
  Description of Artificial Sequence Synthetic oligonucleotide
cggggccggggccggggccg

SEQ ID NO: 35 - DNA, 20 bp, Artificial Sequence
  Description of Artificial Sequence Synthetic oligonucleotide
ggggccggggccggggccgg

SEQ ID NO: 36 - DNA, 20 bp, Artificial Sequence
  Description of Artificial Sequence Synthetic oligonucleotide
cggacggacggacggacgga

SEQ ID NO: 37 - DNA, 20 bp, Artificial Sequence
  Description of Artificial Sequence Synthetic oligonucleotide
ggacggacggacggacggac

SEQ ID NO: 38 - DNA, 20 bp, Artificial Sequence
  Description of Artificial Sequence Synthetic oligonucleotide
gacggacggacggacggacg

SEQ ID NO: 39 - DNA, 20 bp, Artificial Sequence
  Description of Artificial Sequence Synthetic oligonucleotide
acggacggacggacggacgg

SEQ ID NO: 40 - DNA, 20 bp, Artificial Sequence
  Description of Artificial Sequence Synthetic oligonucleotide
ctgcctgcctgcctgcctgc

SEQ ID NO: 41 - DNA, 20 bp, Artificial Sequence
  Description of Artificial Sequence Synthetic oligonucleotide
tgcctgcctgcctgcctgcc

SEQ ID NO: 42 - DNA, 20 bp, Artificial Sequence
  Description of Artificial Sequence Synthetic oligonucleotide
gcctgcctgcctgcctgcct

SEQ ID NO: 43 - DNA, 20 bp, Artificial Sequence
  Description of Artificial Sequence Synthetic oligonucleotide
cctgcctgcctgcctgcctg

SEQ ID NO: 44 - DNA, 20 bp, Artificial Sequence
  Description of Artificial Sequence Synthetic oligonucleotide
agaatagaatagaatagaat

SEQ ID NO: 45 - DNA, 20 bp, Artificial Sequence
  Description of Artificial Sequence Synthetic oligonucleotide
gaatagaatagaatagaatg

SEQ ID NO: 46 - DNA, 20 bp, Artificial Sequence
  Description of Artificial Sequence Synthetic oligonucleotide
aatagaatagaatagaatga

SEQ ID NO: 47 - DNA, 20 bp, Artificial Sequence
  Description of Artificial Sequence Synthetic oligonucleotide
atagaatagaatagaatgaa

SEQ ID NO: 48 - DNA, 20 bp, Artificial Sequence
  Description of Artificial Sequence Synthetic oligonucleotide
tagaatagaatagaatgaat

SEQ ID NO: 49 - DNA, 20 bp, Artificial Sequence
  Description of Artificial Sequence Synthetic oligonucleotide
attctattctattctattct

SEQ ID NO: 50 - DNA, 20 bp, Artificial Sequence
  Description of Artificial Sequence Synthetic oligonucleotide
ttctattctattctattcta

SEQ ID NO: 51 - DNA, 20 bp, Artificial Sequence
  Description of Artificial Sequence Synthetic oligonucleotide
tctattctattctattctat

SEQ ID NO: 52 - DNA, 20 bp, Artificial Sequence
  Description of Artificial Sequence Synthetic oligonucleotide
ctattctattctattctatt

SEQ ID NO: 53 - DNA, 20 bp, Artificial Sequence
  Description of Artificial Sequence Synthetic oligonucleotide
tattctattctattctattc

SEQ ID NO: 54 - DNA, 20 bp, Artificial Sequence
  Description of Artificial Sequence Synthetic oligonucleotide
tggaatggaatggaatggaa

SEQ ID NO: 55 - DNA, 20 bp, Artificial Sequence
  Description of Artificial Sequence Synthetic oligonucleotide
ggaatggaatggaatggaat

SEQ ID NO: 56 - DNA, 20 bp, Artificial Sequence
  Description of Artificial Sequence Synthetic oligonucleotide
gaatggaatggaatggaatg

SEQ ID NO: 57 - DNA, 20 bp, Artificial Sequence
  Description of Artificial Sequence Synthetic oligonucleotide
aatggaatggaatggaatgg

SEQ ID NO: 58 - DNA, 20 bp, Artificial Sequence
  Description of Artificial Sequence Synthetic oligonucleotide
atggaatggaatggaatgga

SEQ ID NO: 59 - DNA, 20 bp, Artificial Sequence
  Description of Artificial Sequence Synthetic oligonucleotide
ttccattccattccattcca

SEQ ID NO: 60 - DNA, 20 bp, Artificial Sequence
  Description of Artificial Sequence Synthetic oligonucleotide
tccattccattccattccat

SEQ ID NO: 61 - DNA, 20 bp, Artificial Sequence
  Description of Artificial Sequence Synthetic oligonucleotide
ccattccattccattccatt

SEQ ID NO: 62 - DNA, 20 bp, Artificial Sequence
  Description of Artificial Sequence Synthetic oligonucleotide
cattccattccattccattc

SEQ ID NO: 63 - DNA, 20 bp, Artificial Sequence
  Description of Artificial Sequence Synthetic oligonucleotide
attccattccattccattcc

SEQ ID NO: 64 - protein, 1368 aa, Streptococcus pyogenes [sequence omitted]

SEQ ID NO: 65 - DNA, 4 bp, Homo sapiens
cctg

SEQ ID NO: 66 - DNA, 12 bp, Homo sapiens
ccccgccccgcg

SEQ ID NO: 67 - DNA, 6 bp, Homo sapiens
ggggcc

SEQ ID NO: 68 - DNA, 6 bp, Homo sapiens
ccccgg

SEQ ID NO: 69 - DNA, 5 bp, Homo sapiens
attct

SEQ ID NO: 70 - DNA, 5 bp, Homo sapiens
tggaa

SEQ ID NO: 71 - DNA, 6 bp, Homo sapiens
ccctct

SEQ ID NO: 72 - RNA, 19 nt, Artificial Sequence
  Description of Artificial Sequence Synthetic oligonucleotide
gcaccucauuugccgaaua

SEQ ID NO: 73 - RNA, 19 nt, Artificial Sequence
  Description of Artificial Sequence Synthetic oligonucleotide
auaaaugaaugguggauca

SEQ ID NO: 74 - RNA, 19 nt, Artificial Sequence
  Description of Artificial Sequence Synthetic oligonucleotide
ccugagcccuaccgaauug

SEQ ID NO: 75 - RNA, 19 nt, Artificial Sequence
  Description of Artificial Sequence Synthetic oligonucleotide
ggacgacccugaccucaaa

SEQ ID NO: 76 - RNA, 19 nt, Artificial Sequence
  Description of Artificial Sequence Synthetic oligonucleotide
ugguuuacaugucgacuaa

SEQ ID NO: 77 - RNA, 19 nt, Artificial Sequence
  Description of Artificial Sequence Synthetic oligonucleotide
ugguuuacauguuguguga

SEQ ID NO: 78 - RNA, 19 nt, Artificial Sequence
  Description of Artificial Sequence Synthetic oligonucleotide
ugguuuacauguuuucuga

SEQ ID NO: 79 - RNA, 19 nt, Artificial Sequence
  Description of Artificial Sequence Synthetic oligonucleotide
ugguuuacauguuuuccua

SEQ ID NO: 80 - DNA, 20 bp, Artificial Sequence
  Description of Artificial Sequence Synthetic oligonucleotide
  Description of Combined DNA/RNA Molecule Synthetic oligonucleotide
ccgccgccgcgctgccgcac

SEQ ID NO: 81 - DNA, 20 bp, Artificial Sequence
  Description of Artificial Sequence Synthetic oligonucleotide
  Description of Combined DNA/RNA Molecule Synthetic oligonucleotide
gcgactatacgcgcaauaug

SEQ ID NO: 82 - DNA, 22 bp, Artificial Sequence
  Description of Artificial Sequence Synthetic primer
caccgacacatgcaacttcgac

SEQ ID NO: 83 - DNA, 22 bp, Artificial Sequence
  Description of Artificial Sequence Synthetic primer
agtagcccacaaaaggcaggtg

SEQ ID NO: 84 - DNA, 22 bp, Artificial Sequence
  Description of Artificial Sequence Synthetic primer
tcacgcaggtcagcaactggtt

SEQ ID NO: 85 - DNA, 19 bp, Artificial Sequence
  Description of Artificial Sequence Synthetic primer
gaaggtgaaggtcggagtc

SEQ ID NO: 86 - DNA, 20 bp, Artificial Sequence
  Description of Artificial Sequence Synthetic primer
gaagatggtgatgggatttc

SEQ ID NO: 87 - DNA, 20 bp, Artificial Sequence
  Description of Artificial Sequence Synthetic oligonucleotide
tcaagttgaggccagaagga

SEQ ID NO: 88 - protein, 4 aa, Unknown
  Description of Unknown ROCK1/2 inhibitor peptide
YSPS

SEQ ID NO: 89 - protein, 7 aa, Unknown
  Description of Unknown ROCK1/2 inhibitor peptide
ERTYSPS

SEQ ID NO: 90 - protein, 12 aa, Unknown
  Description of Unknown ROCK1/2 inhibitor peptide
ERTYSPSTAVRS

SEQ ID NO: 91 - DNA, 10 bp, Artificial Sequence
  Description of Artificial Sequence Synthetic oligonucleotide
tgtctgcctg

SEQ ID NO: 92 - DNA, 12 bp, Homo sapiens
cggcggcggcgg

SEQ ID NO: 93 - DNA, 12 bp, Homo sapiens
ccgccgccgccg

SEQ ID NO: 94 - DNA, 15 bp, Homo sapiens
ccgccgccgccgccg

(19) United States
(12) Patent Application Publication
(10) Pub. No.: US 2024/0173358 A1
(43) Pub. Date: May 30, 2024

(54) Compositions and Methods for the Treatment of Conditions Associated with Nucleotide Repeat Expansion

(71) Applicant: The General Hospital Corporation, Boston, MA (US)

(72) Inventors: Jeannie T. Lee, Boston, MA (US); Hun-goo Lee, Belmont, MA (US)

(21) Appl. No.: 18/282,421

(22) PCT Filed: Mar. 16, 2022
(86) PCT No.: PCT/US2022/020631

§ 371 Date: Sep. 15, 2023

Related U.S. Application Data
(60) Provisional application No. 63/161,841, filed on Mar. 16, 2021.
(60) Provisional application No. 63/161,821, filed on Mar. 16, 2021.

(51) Int. Cl. A61K 35/545; A61K 48/00; C12N 9/22; C12N 15/11; C12N 15/90

(52) U.S. Cl. CPC A61K 35/545; A61K 48/005; C12N 9/22; C12N 15/11; C12N 15/907; C12N 2310/20; C12N 2800/80

(57) ABSTRACT

Described herein are compositions and methods that can be used to contract nucleotide repeat expansion (e.g., CGG) in a gene (e.g., FMR1) in cells of subjects having a condition associated with nucleotide repeat expansion, e.g., subjects with Fragile X syndrome (FXS), FXTAS-Parkinsonism, myotonic dystrophy, and other repeat disorders, by treating the cells using an inactive Cas9 protein and a guide RNA that directs the Cas9 to the gene or by treating the cells using a reactivation cocktail comprising small molecules.

### CLAIM OF PRIORITY

This application claims the benefit of U.S. Provisional Patent Application Ser. No. 63/161,841, filed on Mar. 16, 2021, and 63/161,821, filed on Mar. 16, 2021. The entire contents of the foregoing are hereby incorporated by reference.

### TECHNICAL FIELD

Described herein are compositions and methods that can be used to contract nucleotide repeat expansions, e.g., the CGG repeat in the FMR1 gene in cells of subjects, e.g., subjects with Fragile X syndrome (FXS), FXTAS Parkinsonism, or a disease associated with the expansion of nucleotide repeats, by treating the cells using a catalytically dead CRISPR-Cas9 targeted to the nucleotide repeat of the affected gene and/or by treating the cells using small molecules.

### BACKGROUND

Instability of repetitive DNA sequences within the genome is associated with a number of human diseases, including Fragile X syndrome (FXS), fragile X-associated tremor/ataxia syndrome (FXTAS), Parkinson's Disease (PD), Huntington's disease (HD), myotonic dystrophy, and others. The mutation, sometimes referred to as “nucleotide repeat expansion,” (also called “repeat expansion” or “tandem nucleotide repeat expansion” or, when 3 nucleotides are repeated, “trinucleotide repeat expansion” herein), occurs when the number of triplets/repeats present in a mutated gene is greater than the number found in a normal gene. The expansion of nucleotide repeats is recognized as a major cause of neurological and neuromuscular diseases. See, e.g., Budworth, H. and McMurray, C. T. (2013) Methods Mol Biol. 1010:3-17; Ellerby, Neurotherapeutics 16:924-927 (2019).

The nucleotide repeats are defined as simple sequences of 1-6 nucleotides (or longer) that are repeated multiple times (Budworth, H. and McMurray, C. T. (2013) Methods Mol Biol. 1010:3-17). The threshold at which the repeat expansions become symptomatic varies with the specific disease (Ellerby, L. M. (2019) Neurotherapeutics 16:924-927). For example, Fragile X syndrome (FXS), also termed Martin-Bell syndrome or marker X syndrome, is one of the most common heritable neurodegenerative disorders and is caused by the expansion of CGG nucleotide repeats in the 5′-UTR of the FMR1 gene. There are two types of FXS with distinct pathological lesions depending on the length of the CGG repeats: pre-mutation and full mutation. Very long expansions resulting in over 200 CGG repeats—so-called full mutation (FM)—cause DNA hyper-methylation and consequent epigenetic silencing of the fragile X mental retardation 1 (FMR1) gene, which in turn causes a loss of expression of the fragile X mental retardation protein (FMRP) (McLennan et al., Curr Genomics. 2011 May; 12(3): 216-224). On the other hand, carriers of the pre-mutation alleles (55-200 CGG repeats) exhibit increased FMR1 mRNA levels but normal or lower FMRP expression, implying a different pathological mechanism (Tassone, F., et al., RNA, 2007. 13(4): p. 555-62). Such carriers are asymptomatic earlier in life but develop a condition called FXTAS and presumed to be caused by toxic accumulation of CGG-FMR1 RNA (Hagerman et al., Neurology. 2001 Jul. 10; 57(1):127-30), which is often also diagnosed as Parkinsonism (Niu et al., Parkinsonism Relat Disord. 2014 April; 20(4): 456-459). FMRP is a polyribosome-associated RNA binding protein that regulates the translation of mRNAs from a wide variety of genes, including many genes encoding synaptic proteins (Darnell, J. C., et al., Cell, 2011. 146(2): p. 247-61). Currently, there is no cure for FXS or FXTAS Parkinsonism available, and treatments are limited to alleviating symptoms.

In another example, Huntington's disease (HD), the nucleotide repeat lies within the gene-coding region of the huntingtin (HTT) gene; expansion of the CAG nucleotide repeat creates abnormal proteins with a gain of function as a result of the enlargement of the polyglutamine tract. Unaffected individuals may have roughly 6-29 CAG triplets in both alleles; yet, in HD patients, the disease allele may contain 36 to hundreds of CAG triplets. As the repeat number grows, the growing polyglutamine tract produces an HD gene product (called huntingtin) with increasingly aberrant properties that causes death of brain cells controlling movement (Budworth, H. and McMurray, C. T. (2013) Methods Mol Biol. 1010:3-17).

Currently there are over 40 distinct diseases known to be caused by these nucleotide repeat expansions in DNA sequence, for which there are currently no cures (Ellerby, L. M. (2019) Neurotherapeutics 16:924-927).

### SUMMARY

This application is based, in part, on the discovery that nucleotide repeat expansion (also, “tandem nucleotide repeat expansion” or, when 3 nucleotides are repeated, “trinucleotide repeat expansion” herein) can be retracted and gene function restored by administering an inactive, or catalytically dead, Cas9 protein (“dCas9”) and a guide RNA (“gRNA”) that directs the dCas9 to the gene or nucleotide repeat.

Described herein, inter alia, are methods for contracting an expansion of nucleotide repeats in a gene in a cell. The methods comprise or consist of contacting the cell with or expressing in the cell an inactive Cas9 protein (dCas9) and a guide RNA that directs the dCas9 to the gene and/or nucleotide repeat, in an amount sufficient to reduce the number of nucleotide repeats in the cell. In some embodiments, the expansion is in a fragile X mental retardation 1 (FMR1) gene, wherein the FMR1 gene is inactive due to the presence of expansion of CGG nucleotide repeats in the 5′-UTR of the FMR1 gene, and the number of nucleotide repeats in at least one allele of the gene is reduced after contact with the dCas9 and gRNA, e.g., wherein the number of CGG nucleotide repeats in the 5′-UTR of at least one allele of the FMR1 gene is reduced after contact with the dCas9 and gRNA. In some embodiments, the cell is from a subject who has a disorder caused by the expansion of nucleotide repeats, e.g., a neurodevelopmental or neurodegenerative disorder caused by the expansion of CGG nucleotide repeats, e.g., in the 5′-UTR of the FMR1 gene. In some embodiments, the cell is an induced pluripotent stem cell (iPSC) derived from a somatic cell of a subject who has a disorder caused by the expansion of nucleotide repeats, e.g., a subject who has Fragile X syndrome (FXS). In some embodiments the cell is from a subject who has >200 CGG repeats, or 50-200 CGG repeats.

Also provided herein, inter alia, are populations of cells generated by any of the methods described herein. In some embodiments, the populations of cells are generated by a method comprising contacting a population of cells with or expressing in a population of cells an inactive Cas9 protein and a guide RNA that directs the dCas9 to the gene and/or nucleotide repeat, in an amount sufficient to reduce the number of nucleotide repeats in the cell. In some embodiments, the expansion is in a fragile X mental retardation 1 (FMR1) gene, wherein the FMR1 gene is inactive due to the presence of expansion of CGG nucleotide repeats in the 5′-UTR of the FMR1 gene, and wherein the number of nucleotide repeats in at least one allele of the gene is reduced after contact with dCas9/gRNA, e.g., wherein the number of CGG nucleotide repeats in the 5′-UTR of at least one allele of the FMR1 gene is reduced after contact with the dCas9 and gRNA. In some embodiments, the cells are from a subject who has a disorder caused by the expansion of nucleotide repeats, e.g., a neurodevelopmental or neurodegenerative disorder caused by the expansion of CGG nucleotide repeats, e.g., in the 5′-UTR of the FMR1 gene, thereby creating a population of cells autologous to the subject who has a disorder caused by the expansion of nucleotide repeats or allogenic to a different subject who has a disorder caused by the expansion of nucleotide repeats. In some embodiments, the cells are an induced pluripotent stem cell (iPSC) derived from a somatic cell of a subject who has a disorder caused by the expansion of nucleotide repeats, e.g., a subject who has Fragile X syndrome (FXS), thereby creating a population of iPSC cells. In some embodiments the cells are from a subject who has >200 CGG repeats, or 50-200 CGG repeats.

Described herein, inter alia, are methods for treating a subject who has a condition associated with nucleotide repeat expansion in a gene. These methods comprise or consist of: obtaining iPSC derived from differentiated somatic cells obtained from the subject; exposing the iPSC to, or expressing in the iPSC, an inactive Cas9 protein and a guide RNA that directs the Cas9 to the gene, in an amount sufficient to reduce the number of nucleotide repeats in the cell for a time and under conditions sufficient for reduction of the number of nucleotide repeats; promoting differentiation of the reactivated cells to neural precursor cells or other neuronal cell types; and administering the cells to the brain or spinal cord of the subject (e.g., via ICV, cisternae magna, or intrathecal administration) with a condition associated with nucleotide repeat expansion.

Described herein, inter alia, are methods for treating a subject who has a condition associated with nucleotide repeat expansion in a gene. The methods comprise or consist of administering to the subject, e.g., to the brain or spinal cord of the subject (e.g., via ICV, cisternae magna, or intrathecal administration), a therapeutically effective amount of an inactive Cas9 protein and a guide RNA that directs the dCas9 to the gene or the nucleotide repeats, in an amount sufficient to reduce the number of nucleotide repeats in the cell.

Also described herein, inter alia, are methods for contracting nucleotide repeats in a gene a living cell, e.g., a cell having a number of repeats above a reference number or in a reference range. The method comprise or consist of contacting the cell with an inactive Cas9 protein and a guide RNA that directs the Cas9 to the gene or the nucleotide repeats, in an amount sufficient to reduce the number of nucleotide repeats in the cell. In some embodiments, the reference number is 30 repeats, 40 repeats, 50 repeats, or 200 repeats; or the range is 30-100 repeats, 40-200 repeats, or 50-200 repeats. In some embodiments, the reference number is the number of nucleotide repeats in a healthy cell not having a condition associated with nucleotide repeat expansion or a control cell. In some embodiments, the reference number is 0-30 repeats, 0-40, or 0-50 repeats. In some embodiments, the cell is in a living subject. In some embodiments, the cell is in the brain of the subject. In some embodiments, the cell is an iPSC derived from differentiated somatic cell obtained from a subject who has a condition associated with nucleotide repeat expansion. In some embodiments, the dCas9 and gRNA is administered to the CNS, e.g., brain or spinal cord of the subject (e.g., via ICV, cisternae magna, or intrathecal administration), or is administered systemically to the subject.

In some embodiments of any of the methods described herein, the nucleotide repeats comprise any one or more repeats listed in Table A, e.g., CGG repeats, GGC repeats, CAG repeats, CTG repeats, GAA repeats, CCCCGG repeats, CCTG repeats, ATTCT repeats, CAAA repeats, and TGGAA repeats.

In some embodiments of any of the methods described herein, the condition associated with nucleotide repeat expansion is any one or more of FXS, FXTAS, PD, HD, myotonic dystrophy, or any known disorder with repeat expansion, e.g., as listed in Table A.

Also described herein are methods for preparing a population of neural precursor cells or other neuronal cell type with decreased nucleotide repeats. The methods comprise or consist of: obtaining iPSC derived from differentiated somatic cells obtained from a subject who has a nucleotide repeat expansion, e.g., who has a condition associated with nucleotide repeat expansion; exposing the iPSC to an inactive Cas9 protein and a guide RNA that directs the dCas9 to the gene for a time and under conditions sufficient for reduction of the number of nucleotide repeats; and promoting differentiation of the iPSC to neural precursor cells or other neuronal cell type, thereby promoting differentiation of the reactivated cells to neural precursor cells or other neuronal cell types.

Provided herein, inter alia, are populations of neural precursor cells or other neuronal cell type with decreased nucleotide repeats prepared using any of the methods described herein. In some embodiments, a method of preparing a population of neural precursor cells or other neuronal cell type with decreased nucleotide repeats comprises: obtaining iPSC derived from differentiated somatic cells obtained from a subject who has a condition associated with nucleotide repeat expansion; exposing the iPSC to an inactive Cas9 protein and a guide RNA that directs the Cas9 to the gene for a time and under conditions sufficient for reduction of the number of nucleotide repeats; and promoting differentiation of the iPSC to neural precursor cells or other neuronal cell type, thereby promoting differentiation of the reactivated cells to neural precursor cells or other neuronal cell types.

This application is also based, in part, on the discovery that nucleotide repeat expansion (also called “repeat expansion” or “tandem nucleotide repeat expansion” or, when 3 nucleotides are repeated, “trinucleotide repeat expansion” herein) can be retracted and gene function restored by a combination of small molecules, wherein the combination of small molecules is any two or more of a MEK inhibitor, a Raf inhibitor, a ROCK1 inhibitor, a GSK-3β inhibitor, and a Src Inhibitor.

Described herein, inter alia, are methods for reactivating an inactive gene in a living cell, wherein the gene is inactive due to the presence of expansion of nucleotide repeats, optionally wherein the inactive gene is fragile X mental retardation 1 (FMR1), wherein the gene is inactive due to the presence of expansion of nucleotide repeats (e.g., CGG nucleotide repeats in the 5′-UTR of the FMR1 gene). The methods comprise or consist of contacting the cell with a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor; or
(v) a MEK inhibitor, a Raf inhibitor, a ROCK1 inhibitor, a GSK-3β inhibitor, and a Src Inhibitor, for a time and under conditions sufficient for reactivation of the gene.

In some embodiments, the number of nucleotide repeats in at least one allele of the gene is reduced after contact with the reactivation cocktail, e.g., wherein the number of CGG nucleotide repeats in the 5′-UTR of at least one allele of the FMR1 gene is reduced after contact with the reactivation cocktail. In some embodiments, the cell is from a subject who has a disorder caused by the expansion of nucleotide repeats, e.g., a neurodevelopmental or neurodegenerative disorder caused by the expansion of CGG nucleotide repeats in the 5′-UTR of the FMR1 gene. In some embodiments, the cell is an induced pluripotent stem cell (iPSC) derived from a somatic cell of a subject who has a disorder caused by the expansion of nucleotide repeats, e.g., a subject who has Fragile X syndrome (FXS). In some embodiments, the cell is from a subject who has >200 CGG repeats, or 50-200 CGG repeats.

Also provided herein, inter alia, are populations of cells generated by any of the methods described herein. In some embodiments, the populations of cells are generated by a method comprising contacting a population of cells with a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor; or
(v) a MEK inhibitor, a Raf inhibitor, a ROCK1 inhibitor, a GSK-3β inhibitor, and a Src Inhibitor. In some embodiments, the number of nucleotide repeats in at least one allele of the gene is reduced after contact with the reactivation cocktail, e.g., wherein the number of CGG nucleotide repeats in the 5′-UTR of at least one allele of the FMR1 gene is reduced after contact with the reactivation cocktail. In some embodiments, the population of cells is from a subject who has a disorder caused by the expansion of nucleotide repeats, e.g., a neurodevelopmental or neurodegenerative disorder caused by the expansion of CGG nucleotide repeats in the 5′-UTR of the FMR1 gene. In some embodiments, the population of cells is an induced pluripotent stem cell (iPSC) derived from a somatic cell of a subject who has a disorder caused by the expansion of nucleotide repeats, e.g., a subject who has Fragile X syndrome (FXS). In some embodiments, the population of cells is from a subject who has >200 CGG repeats, or 50-200 CGG repeats.

Described herein, inter alia, are methods of treating a subject who has a condition associated with nucleotide repeat expansion. The methods comprise or consist of: obtaining iPSC derived from differentiated somatic cells obtained from the subject; exposing the iPSC to a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor; or
(v) a MEK inhibitor, a Raf inhibitor, a ROCK1 inhibitor, a GSK-3β inhibitor, and a Src Inhibitor, for a time and under conditions sufficient for reduction of the number of nucleotide repeats;
promoting differentiation of the reactivated cells to neural precursor cells or other neuronal cell types; and administering the cells to the brain or spinal cord of the subject (e.g., via ICV, cisternae magna, or intrathecal administration) with a condition associated with nucleotide repeat expansion.

Also described herein are methods for treating a subject who has a condition associated with nucleotide repeat expansion. The methods comprise or consist of administering to the subject, e.g., to the brain or spinal cord of the subject (e.g., via ICV, cisternae magna, or intrathecal administration), a therapeutically effective amount of a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor;
(v) a MEK inhibitor, a Raf inhibitor, a ROCK1 inhibitor, a GSK-3β inhibitor, and a Src Inhibitor.

Described herein, inter alia, are methods for contracting nucleotide repeats in a living cell, e.g., a cell having a number of repeats above a reference number or in a reference range. The methods comprise or consist of contacting the cell with a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor; or
(v) a MEK inhibitor, a Raf inhibitor, a ROCK1 inhibitor, a GSK-3β inhibitor, and a Src Inhibitor, for a time and under conditions sufficient for contraction of the nucleotide repeats.

In some embodiments, the reference number is 30 repeats, 40 repeats, 50 repeats, or 200 repeats; or the range is 30-100 repeats, 40-200 repeats, or 50-200 repeats. In some embodiments, the reference number is the number of nucleotide repeats in a healthy cell not having a condition associated with nucleotide repeat expansion or a control cell. In some embodiments, the reference number is 0-30 repeats, 0-40, or 0-50 repeats. In some embodiments, the cell is in a living subject. In some embodiments, the cell is in the brain of the subject. In some embodiments, the cell is an iPSC derived from differentiated somatic cell obtained from a subject who has a condition associated with nucleotide repeat expansion. In some embodiments, the reactivation cocktail is administered to the CNS, e.g., brain or spinal cord of the subject (e.g., via ICV, cisternae magna, or intrathecal administration), or is administered systemically to the subject.

In some embodiments of any of the methods described herein, the nucleotide repeats comprise any one or more repeats listed in Table A, e.g., CGG repeats, GGC repeats, CAG repeats, CTG repeats, GAA repeats, CCCCGG repeats, CCTG repeats, ATTCT repeats, CAAA repeats, and TGGAA repeats.

In some embodiments of any of the methods described herein, the condition associated with nucleotide repeat expansion is any one or more of FXS, FXTAS, PD, HD, myotonic dystrophy, or any known disorder with repeat expansion, e.g., as listed in Table A.

Also described herein are methods for preparing a population of neural precursor cells or other neuronal cell type with decreased nucleotide repeats. The methods comprise or consist of: obtaining iPSC derived from differentiated somatic cells obtained from a subject who has a nucleotide repeat expansion, e.g., who has a condition associated with nucleotide repeat expansion; exposing the iPSC to a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor;
(v) a MEK inhibitor, a Raf inhibitor, a ROCK1 inhibitor, a GSK-3β inhibitor, and a Src Inhibitor,
for a time and under conditions sufficient for reduction of the number of nucleotide repeats, and promoting differentiation of the iPSC to neural precursor cells or other neuronal cell type thereby promoting differentiation of the reactivated cells to neural precursor cells or other neuronal cell types.

Provided herein, inter alia, are populations of neural precursor cells or other neuronal cell type with decreased nucleotide repeats prepared using any of the methods described herein. In some embodiments, the method of preparing a population of neural precursor cells or other neuronal cell type with decreased nucleotide repeats, the method comprising obtaining iPSC derived from differentiated somatic cells obtained from a subject who has a nucleotide repeat expansion, e.g., who has a condition associated with nucleotide repeat expansion; exposing the iPSC to a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor; or
(v) a MEK inhibitor, a Raf inhibitor, a ROCK1 inhibitor, a GSK-3β inhibitor, and a Src Inhibitor,
for a time and under conditions sufficient for reduction of the number of nucleotide repeats; and promoting differentiation of the iPSC to neural precursor cells or other neuronal cell type, thereby promoting differentiation of the reactivated cells to neural precursor cells or other neuronal cell types.

In some embodiments, any of the methods or treatments described herein are used in combination with an inhibitor of DNA methyltransferase (DNMT), as described herein. In some embodiments, a useful DNMT inhibitor is at least one of is RG108, 5-azacytidine, decitabine, Zebularine, procainamide, procaine, psammaplin A, sinefungin, temozolomide, OM173-alphaA, DNMT3A-binding protein, theaflavin 3,3′-digallate, 1-Hydrazinophthalazine, SGI-1027, hydralazine, NSC14778, Olsalazine, Nanaomycin, SID 49645275, Δ2-isoxazoline, epigallocatechin-3-gallate (EGCG), MG98, SGI-110, SGI-1027, SW155246, SW15524601, SW155246-2, or DZNep, an ASO targeting DNMT, optionally comprising SEQ ID NO: 87, TCAAGTTGAGGCCAGAAGGA, or an siRNA targeting DNMT, optionally comprising SEQ ID NOs: 72-75.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1J. FMR1 reactivation by cellular reprogramming to a naive state. 1A) Electrophoresis of PCR products from repeat PCR (RPT-PCR) assays examined repeat length and DNA methylation status in various patient cells lines, as indicated. Addition (+) of HpaII to genomic DNA prior to RPT-PCR tested methylation status. RPT-PCR product lengths (black) and corresponding CGG repeat copy numbers (red) are shown. 1B) FMR1 mRNA quantitation from the cells shown in (1A). Expression levels normalized to WT hiPSC levels. A schematic map depicting FMR1 gene shown above the bar graph. 1C) Schematic depiction of experimental timeline showing periods of acclimation to mTeSR, RSeT, and 5i media, along with accompanying changes in FMR1 mRNA levels after 12 days of 5i treatment. 1D) Western blot analysis of FMRP in WT hiPSC versus FXS hESC grown in indicated media. GAPDH served as loading control. 1E) Time course analysis of CGG repeat length and DNA methylation status (HpaII+/−) in FXS full mutation clone, 848-1c, between 0-36 days of 5i treatment. Gel electrophoresis of RPT-PCR products shown, with RPT-PCR product lengths (black) and corresponding repeat copy numbers (red). 1F) Bioanalyzer quantitation of CGG repeat lengths in a 0-36 day time course. RPT-PCR product lengths (black) and corresponding repeat copy numbers (red) are shown. 1G) Box plots for quantitation of repeats in high copy number (210-277×CGGs) and low copy number (44-110×CGGs) ranges after 0-36 days of 5i treatment. 1H) To verify contraction, RPT-PCR assays of hiPSC clones from 848-1h were performed after further subcloning following 5i treatment, as shown in the schematic depicting the timeline. Six subclones and their repeat numbers are shown. 1I) Quantitation of FMR1 mRNA levels in subclones of 848-1h in (H) by RT-qPCR, shown relative to WT hiPSC levels. 1J) Western blot analysis of FMRP levels in the indicated 848-1h subclones. Tubulin, loading control. CGG repeat numbers shown below each subclone.

FIGS. 2A-2E. FMR1 reactivation and repeat contraction by MEK and BRAF inhibition. 2A) Table of kinase inhibitors, abbreviations, and concentrations used in 5i media. 2B) Determination of active compounds in 5i media for FMR1 reactivation by testing single, double, and triple combinations of inhibitors in 848-1c cells. FMR1 RNA levels determined by RT-qPCR. 2C) Box plots showing the DNA methylation levels at FMR1 promoter CpG island determined by pyrosequencing assay after treating 848-1c cells with RSet, 3i (PSR), 4i (PISR), or 5i. P value determined by the Student t-test. ****, P<0.0001. 2D) Bioanalyzer quantitation of CGG repeat length/copy number after treatment with various combinations indicated for 12 days. 2E) Box plots quantitation of repeats in high copy number (210-277×CGGs) and low copy number (44-110×CGGs) ranges after 12 days of treatment with indicated combinations. P value determined by the Student t-test. *, P<0.05, ***, P<0.001, ****, P<0.0001.

FIGS. 3A-3I. Repeat contraction attributed to DNA demethylation and inhibiting DNMT1 potentiated the 5i effect. 3A) MeDIP-qPCR assay using anti-5 mC antibodies measured DNA methylation levels at FMR1 in 848-1c iPSC grown in RSeT media versus 9 days of 5i. P-value determined by the Student t-test. ****, P<0.0001. 3B) Pyrosequencing analysis of DNA methylation at CpG islands in FMR1 promoter with single nucleotide resolution in WT versus FXS cells grown in various media. 3C) ChIP-qPCR assay at FMR1 promoter in 848-1c cells grown in RSeT media versus 6 days of 5i. P value determined by the Student t-test (*, P<0.05). IgG ChIP, negative control. 3D) Bioanalyzer analysis of CGG repeat length distribution after DNMT1 knockdown. 3E) Box plots quantitation of repeats in high copy number (210-277×CGGs) and low copy number (44-110×CGGs) ranges after 6 days of siDNMT1 knockdown. P-value determined by the Student t-test. ****, P<0.0001. 3F) RT-qPCR of FMR1 expression in 848-1c cells treated with siCtrl versus siDNMT1 in 5i for 6 days. P-value determined by the Student t-test. *, P<0.05. 3G) FMR1 reactivation after 848-1c cells were exposed to dCas9-Tet1, dCas9-Tet-DEAD, or only gRNA for 0-27 days in mTeSR. P-value determined by t-test. ns, not significant, *, P<0.05, **, P<0.01, ****, P<0.0001. 3H) Bioanalyzer traces of repeat length/copy number distribution after 27 days of treatment. 3I) Bioanalyzer signals for ranges of 210-277 CGGs versus 44-110 CGGs for each condition as indicated. P<0.0001, t-test.

FIGS. 4A-4L. Site-specific R-loop formation triggered CGG contraction. 4A) DRIP assay at the FMR1 5′UTR in 848-1c iPSC after 6 days of 5i. Treatment with RNaseH (RH+) abolished DRIP signals. *, P<0.05, t-test. ns, not significant. 4B) Bioanalyzer profiles for RPT-PCR product length and estimated CGG repeat copy number in 848-1c iPSC after 12 days treatment with FMR1 Gapmer as compared to control Gapmer treatment. ASO designs depicted above. 4C) Box plots quantitation of repeats in high copy number (210-277×CGGs) and low copy number (44-110×CGGs) ranges after indicated treatment for 12 days. t-test: ***, P<0.001, ****, P<0.0001. 4D) DRIP assay at the FMR1 5′UTR in 848-1c iPSC treated as shown in schematic, with gCGG alone, dCas9+gCGG, or dCas9−RH+gCGG for 6 days. t-test: *, P<0.05, ***, P<0.001, ns, not significant. 4E) Gel electrophoresis of CGG RPT-PCR products+/−HpaII digestion in 848-1c iPSC, treated for 6 days as shown in (4D). 4F) Bioanalyzer analysis of RPT-PCR product length and estimated CGG repeat copy number for the samples depicted in (4D-4E). 4G) Box plots for quantitation of repeats in high copy number (210-277×CGGs) and low copy number (44-110×CGGs) ranges after indicated treatment for 12 days. t-test: ****, P<0.0001. 4H) 5 hmC MeDIP assay at the FMR1 5′UTR in 848-1c iPSC treated, with gCCG alone, dCas9+gCGG, or dCas9−RH+gCGG for 6 days. t-test: *, P<0.05, ns, not significant. 4I) RT-qPCR of FMR1 mRNA levels for corresponding samples shown in (4A-4E). t-test: **, P<0.01; ***, P<0.001. 4J) Schematic timeline for experiments to test sufficiency of dCas9-targeted R-loops in 848-1c hiPSCs without accompanying 5i treatment, and RT-qPCR of FMR1 mRNA levels following 24-day exposure to gCGG alone, dCas9+gCGG, or dCas9−RH+gCGG. Cells were grown without 5i. t-test: *, P<0.05. 4K) Box plots for quantitation of repeats in high copy number (210-277×CGGs) and low copy number (44-110×CGGs) ranges after indicated treatment shown in (4J). T-test: ****, P<0.0001. 4L) Pyrosequencing examined DNA methylation status at FMR1 promoter-associated CpG island for samples shown in (4J-4K).

FIGS. 5A-5I. Strong on-target CGG contraction and gene reactivation triggered by R-loop formation. 5A) Schematic depicting a map of FMR1 gene and design of FMR1-specific gRNAs (gNHG2, gNHG3). gNHG2 and gNHG3 spans both unique sequence and CGG repeats. Scrambled gRNA (gScr), negative control. 5B) Bioanalyzer profiles for CGG length and repeat copy number for 848-1c hiPSCs targeted as shown in (5A) for 36 days in mTeSR regular media. 5C) Box plots quantitation of repeats in high copy number (210-277×CGGs) and low copy number (44-110×CGGs) ranges after indicated treatment per (5A) above. t-test: ***, P<0.001, ****, P<0.0001. 5D) Pyrosequencing examined DNA methylation status at FMR1 promoter-associated CpG island for samples targeted as indicated per (5A-5B) above. 5E) RT-qPCR of FMR1 mRNA levels for samples shown in (5A-5D). t-test: ****, P<0.0001. ns, not significant. 5F) Western blot analysis of FMRP levels for the experiments in (5A-5E). Tubulin, loading control. 5G) Volcano plot of transcriptomic analysis of 848-1c hiPSCs targeted by dCas9+gNHG3 versus dCas9+gScr. Log2 fold-change for differentially expressed (DE) genes were plotted against statistical significance (−Log₁₀P). Red dots, significantly changed genes. 5H) Integrated Genome browser (IGV) views for two biological replicates (rep1, rep2) of the RNA-seq analysis of dCas9+gScr versus dCas9+gNHG3. Three genes with CGG tracts are shown. Scale is indicated in brackets. 5I) RPT-PCR results showed absence of off-target CGG contraction at RGPD2 (middle plot) and RGPD1 (bottom plot) following dCas9 targeting by gScr, gNHG3, and gCGG in 848-1c FXS hiPSCs.

FIGS. 6A-6E. Positive feedback loop for FMR1 reactivation and DNA mismatch repair mechanism-mediated repeat contraction. 6A) Diagram depicting R-loop formation and two major DNA repair pathways known to be related to R-loop. 6B) ChIP-qPCR for γ-H2AX at FMR1 locus in 848-1c cells grown in either RSeT or 5i media. 6C) Bioanalyzer profiles for RPT-PCR product length and estimated CGG repeat copy number in 848-1c iPSC after 12 days treatment with siMSH2 or siCSB, as compared to control siRNA treatment. Cells were grown in 5i media. 6D) Box plots quantitation of repeats in high copy number (210-277×CGGs) and low copy number (44-110×CGGs) ranges after indicated treatment for 12 days. t-test: ***, P<0.001, ****, P<0.0001. 6E) Schematic depiction of a positive feedback cycle of DNA demethylation, transcription, R-loop formation, and repeat contraction driving reactivation of FMR1.

FIGS. 7A-7C. FMRP restoration and single-cell cloning of FXS hiPSC and hESC lines. 7A) Establishment of multiple single cell clones for FXS full mutation hiPSC line 848 and hESC line WCMC37 (“37”). 7B) Bioanalyzer profiles of RPT-PCR product length and estimated CGG repeat copy number for 848-1c hiPSCs on day 0 and day 6 of 5i treatment. 7C) RPT-PCR analysis of additional FXS iPSC and hESC clones after 0, 27 or 36 day 5i treatment.

FIGS. 8A-8B. No repeat contraction detected in WT and pre-mutation cells by 5i. 8A) Gel electrophoresis of CGG RPT-PCR products+/−HpaII digestion for pre-mutation hiPSC clone 131-1a, grown in RSeT or treated for 27 days with 5i. 8B) Gel electrophoresis of CGG RPT-PCR products+/−HpaII digestion for WT hiPSC line 8330, grown in mTeSR, RSeT, or treated for 12 days with 5i.

FIGS. 9A-9B. DNA de-methylation by MEKi and BRAFi. 9A) DNA methylation status at FMR1 promoter CpG island determined by pyrosequencing assay after treating 848-1c cells with RSeT, 3i (PSR), 4i (PISR), or 5i. 9B) Box plots for quantification of the DNA methylation levels at FMR1 promoter CpG island determined by pyrosequencing assay.

FIGS. 10A-10C. Partial depletion of DNMT1 alone not sufficient to trigger repeat contraction. 10A) RT-qPCR of DNMT1 mRNA levels in the 848-1c cells treated with siCtrl or siDNMT1 for 6 days in RSeT media. t-test: **, P<0.01. 10B) RT-qPCR of FMR1 mRNA levels in 848-1c cells treated with siCtrl or siDNMT1 for 6 days in RSeT media. 10C) RT-qPCR of DNMT1 mRNA levels in 848-1c cells treated with siCtrl or siDNMT1 for 6 days in 5i media. t-test: **, P<0.01.

FIGS. 11A-11C. CGG repeat contraction by 5i dependent upon R-loop, and R-loop formation triggered active DNA demethylation. 11A) Gel electrophoresis of CGG RPT-PCR products in 848-1c iPSC treated for 12 days with FMR1 gapmer (cleaving) or control Gapmer. 11B) RT-qPCR of MSH2 mRNA levels in the 848-1c cells treated with siCtrl or siMSH2 for 6 days in 5i media. t-test: ****, P<0.0001. 11C) 5 hmC MeDIP assay at the FMR1 5′UTR in 848-1c iPSC treated with gCCG alone, dCas9+gCGG, or dCas9−RH+gCGG for 6 days. t-test: *, P<0.05, ns, not significant.

FIGS. 12A-12B. R-loop formation by dCas9 and CGG triggered R-loop dependent CGG repeat contraction and DNA demethylation in FMR1 locus without 5i treatment. 12A) Bioanalyzer traces of repeat length/copy number distribution in 848-1c cells exposed to 24 days of dCas9+gCGG, dCas9−RNaseH+gCGG, or gCGG alone in mTeSR media for 24 days. 12B) Quantitation of pyrosequencing to examine DNA methylation status at FMR1 promoter-associated CpG island for the profiles in FIG. 12A. t-test: ****, P<0.0001.

FIGS. 13A-13E. R-loop formation by dCas9 with gCGG and gNHG3 can trigger DNA demethylation in FMR1 locus, and the repeat contraction and transcription activation were FMR1 locus specific. 13A) Box plot quantitation of pyrosequencing examined DNA methylation status at FMR1 promoter-associated CpG island for 848-1c cells exposed to 36 days of dCas9+gCGG, dCas9−RNaseH1+gCGG, or gCGG alone in mTeSR media. t-test: ****, P<0.0001. 13B) Gel electrophoresis of RPT-PCR products using a distinct primer pair for CGG repeat tracts in RGPD1 promoter region. FXS iPS 848-1c cells exposed to dCas9 targeted by gScr, gNHG3, or gCGG for 36 days in mTeSR media. 13C) Genome browser (IGV) views for two biological replicates (rep1, rep2) of the RNA-seq analysis of dCas9+gScr versus dCas9+gNHG3. Two negative control genes with CGG tracts at the 5′ UTR were shown. Scale is indicated in brackets. 13D) Gel electrophoresis of FMR1 RPT-PCR products+/−HpaII pre-digestion for WT hiPSC line 8330 exposed to dCas9 targeted by gScr, gNHG3, or gCGG for 36 days in mTeSR media. 13E) Gel electrophoresis of RPT-PCR products+/−HpaII pre-digestion showed absence of off-target CGG contraction at AFF2 following 25 days of 5i treatment in 848-1c FXS hiPSCs.

FIGS. 14A-14B. DMPK and SIX5 reactivation in DM1 cells via small molecules inhibitors. 2A) Bar graph of DMPK mRNA level (normalized to GAPDH levels). DM1-202 or DM2-221 cells were treated with 50:50 5i and RSET, 50:50 3i and RSET, RG108, or DMSO (control), and DMPK mRNA levels were measured by RT-qPCR at day 8 and day 12. 2B) Bar graph of SIX5 mRNA level (normalized to GAPDH levels). DM1-202 or DM2-221 cells were treated with 50:50 5i and RSET, 50:50 3i and RSET, RG108, or DMSO (control), and DMPK mRNA levels were measured by RT-qPCR at day 8 and day 12.

FIG. 15. DMPK and SIX5 reactivation in DM1 cells via small molecules inhibitors. Bar graph showing RNA levels of DMPK, SIX5, DMWD, MBNL, and CUGBP measured for 6 cells types: WR iPS 8330, DM1-115, DM1-202, DM1-203, DM2-220, and DM2-221 (normalized to iPS 8330).

### DETAILED DESCRIPTION

The molecular mechanism of repeat expansion and contraction in a number of genes, e.g., the FMR1 gene, is largely unknown. A few models for nucleotide repeat expansion were suggested (see, e.g., Mirkin, S. M., Nature, 2007. 447(7147): p. 932-40; Moore, H., et al., Proc Natl Acad Sci USA, 1999. 96(4): p. 1504-9; Usdin, K., N. C. House, and C. H. Freudenreich, Crit Rev Biochem Mol Biol, 2015. 50(2): p. 142-67) including DNA polymerase slippage and Okazaki fragment displacement during DNA replication. Misalignment of the repeat tracts during double-strand brake repair was also suggested as a possible model to cause repeat expansion and contraction. Repeat expansion mostly occurs on maternal transmission through pre-mutation females, and larger repeats carry higher risks of expansion (Rousseau, F., et al., N Engl J Med, 1991. 325(24): p. 1673-81).

Understanding the cellular mechanisms that promotes repeat expansion and contraction could be extremely valuable towards formulating a strategy for treating diseases associated with nucleotide repeat expansion, e.g., an FXS therapeutic, one that would enable permanent reactivation of an affected gene, e.g., the silent FMR1 gene, in a patient by shortening the repeat expansion, e.g., the CGG repeat.

As shown herein, surprisingly, it is possible inter alia to contract the CGG repeat by treating cells using a specific protocol involving small molecules and that at least the FMR1 gene can be concurrently reactivated in full. The FMR1 gene was completely silenced in human induced pluripotent stem (iPS, iPSC, hPSCs, iPSCs) and embryonic stem (ES) cell lines from FXS full mutation patients. By treating these cells with sets of small molecules, we demonstrated de-repressed FMR1 gene transcription and a very strong increase in the protein FMRP levels. In these cells, we also demonstrated that the long CGG repeat was contracted and DNA methylation was decreased.

As shown herein, it was possible to fully reactivate the FMR1 gene, inter alia, and contract the CGG repeat by inducing epigenetic state change either towards naïve state or targeted DNA de-methylation on FMR1 locus in FXS hPSCs. Without wishing to be bound by theory, since H3K9me3 didn't show a decrease and DNA de-methylation by Tet1 recruitment to FMR1 locus was sufficient to trigger both FMR1 gene reactivation and CGG repeat contraction, DNA de-methylation is thought to be a potential cause for the observed phenomena. A significant FMR1 transcription reactivation (about ˜50% of normal cell level) preceded the CGG repeat shortening, implying that it is not a major cause of the observed increase of FMR1 gene transcription although it might contribute further FMR1 gene upregulation at later phase of observed reactivation.

The present methods can be utilized to correct patient-derived iPSCs ex vivo using small molecules to trigger repeat contraction (e.g., CGG repeat contraction), and a patient's own corrected cells can be administered to treat FXS in the subject. In addition, the compositions described herein can be administered directly to the subject, e.g., to the CNS of the subject.

As the epigenetic reprogramming by the optimized small molecule treatment re-awakens the native FMR1 gene carried within the patient's own cells and, more importantly, permanently shrinks the long CGG repeat by cell-intrinsic DNA repair mechanism without introducing exogenous factors, this reactivation strategy can be utilized for a cell therapy treatment regimen for iPSC-based cell autonomous therapy for FXS. Stem cell therapy is emerging as a treatment option for various severe diseases. For example, the first successful clinical trial for an autonomous cell-based Parkinson's treatment was reported in 2020, treating by engrafting dopaminergic neuronal progenitor cells derived from the patient's iPSCs (Schweitzer, J. S., et al., N Engl J Med, 2020. 382(20): p. 1926-1932). The use of patient-specific stem cells would circumvent immune issues normally associated with transplant. Moreover, inter alia, our approach would bypass the safety concerns associated with gene editing or gene therapy relating to the introduction of foreign genetic material and the possibility of off-target mutagenesis.

As this approaches directly corrects the actual cause by permanently shortening the long CGG repeats, it differs from most of the unsuccessful FXS studies that focused on the downstream pathway dysfunctions in FXS, including recent failed clinical trials with metabotropic glutamate receptor 5 (mGluR5) antagonists (Erickson, C. A., et al., J Neurodev Disord, 2017; 9: 7.). Since FMRP modulates many different levels of pathways, FXS exhibits molecular, synaptic, and circuit levels of dysfunctions, which cannot be easily corrected via a downstream method. As the present approach reactivates or restores the function of the FMR1 by dealing with the upstream cause, the long CGG repeats, it has much greater potential to successfully alleviate a number of symptoms of FXS.

As shown herein, surprisingly, it is possible inter alia to contract the CGG repeat by treating cells using a catalytically dead CRISPR-Cas (dCas) protein targeted to the FMR1 locus; without wishing to be bound by theory, it is believed that this results in formation of an R-loop and initiation of double-strand break repair (DSBR), which in turn leads to contraction of the CGG repeat and reactivation of the FMR1 gene at therapeutic levels. The FMR1 gene was completely silenced in human induced pluripotent stem (iPS, hPSCs, iPSCs) and embryonic stem (ES) cell lines from FXS full mutation patients. By treating these cells with dCas9, we demonstrated de-repressed FMR1 gene transcription and a very strong increase in the protein FMRP levels. In these cells, we also demonstrated that the long CGG repeat was contracted and DNA methylation was decreased. In addition, premutation cells with intermediate length of CGG repeats also showed contraction by dCas9 targeting combined with naïve state media treatment, indicating this method can be utilized for other repeat-mediated diseases including, but not limited to, FXTAS, PD, and HD.

Also as shown herein, surprisingly, it is possible to contract the CGG repeat by treating cells using a specific protocol involving small molecules and that the FMR1 gene can be concurrently reactivated in full. The FMR1 gene was completely silenced in human induced pluripotent stem (iPS, hPSCs, iPSCs) and embryonic stem (ES) cell lines from FXS full mutation patients. By treating these cells with sets of small molecules, we demonstrated de-repressed FMR1 gene transcription and a very strong increase in the protein FMRP levels. In these cells, we also demonstrated that the long CGG repeat was contracted and DNA methylation was decreased.

As shown herein, it was possible to fully reactivate the FMR1 gene and contract the CGG repeat by inducing epigenetic state change either towards naïve state or targeted DNA de-methylation on FMR1 locus in FXS hPSCs. Without wishing to be bound by theory, since H3K9me3 didn't show a decrease and DNA de-methylation by Tet1 recruitment to FMR1 locus was sufficient to trigger both FMR1 gene reactivation and CGG repeat contraction, DNA de-methylation is thought to be the cause for the observed phenomena. A significant FMR1 transcription reactivation (about ˜50% of normal cell level) preceded the CGG repeat shortening, implying that it is not a major cause of the observed increase of FMR1 gene transcription although it might contribute further FMR1 gene upregulation at later phase of observed reactivation. The epigenetic reprogramming by the optimized small molecule treatment re-awakens the native FMR1 gene carried within the patient's own cells and, more importantly, permanently shrinks the long CGG repeat by cell-intrinsic DNA repair mechanism without introducing exogenous factors.

The present methods can be utilized to correct patient-derived iPSCs ex vivo using (i) dCas9 and a targeting guide RNA and/or (ii) a reactivation cocktail of small molecules to trigger trinucleotide, e.g., CGG, repeat contraction, and the patient's own corrected cells can then be administered to treat a disease associated with repeat expansion, e.g., FXS, in the subject. In addition, the compositions described herein can be administered directly to the subject, e.g., to the CNS of the subject.

The repeat contraction strategies described herein can be utilized directly (e.g., by delivering the dCas9/gRNA or reactivation cocktail to the target tissues of subject), or for a cell therapy treatment regimen for iPSC-based cell autonomous therapy for FXS. Stem cell therapy is emerging as a treatment option for various severe diseases. For example, the first successful clinical trial for an autonomous cell-based Parkinson's treatment was reported in 2020, treating by engrafting dopaminergic neuronal progenitor cells derived from the patient's iPSCs (Schweitzer, J. S., et al., N Engl J Med, 2020. 382(20): p. 1926-1932). The use of patient-specific stem cells would circumvent immune issues normally associated with transplant. Moreover, this approach would bypass the safety concerns associated with gene editing or gene therapy relating to the introduction of foreign genetic material and the possibility of off-target mutagenesis.

As this approaches directly corrects the actual cause by permanently shortening the long CGG repeats, it differs from most of the unsuccessful FXS studies that focused on the downstream pathway dysfunctions in FXS, including recent failed clinical trials with metabotropic glutamate receptor 5 (mGluR5) antagonists (Erickson, C. A., et al., J Neurodev Disord, 2017; 9:7). Since FMRP modulates many different levels of pathways, FXS exhibits molecular, synaptic, and circuit levels of dysfunctions, which cannot be easily corrected via a downstream method. As the present approach restore the function of the FMR1 gene by dealing with the upstream cause, the long CGG repeats, it has much greater potential to successfully alleviate a number of symptoms of FXS.

### CRISPR/Cas Targeting of Nucleotide Repeats

The present methods include using catalytically dead versions of Cas9 (dCas9) which are derived from Cas9 proteins, e.g., SpCas9 or SpCas9 variants, e.g., variants with altered PAM specificity or that have improved on-target editing capabilities. These methods can be applied to other CRISPR-Cas proteins, including other Cas9 orthologs with various levels of basal activity (SaCas9, St1Cas9, St3Cas9, NmeCas9, Nme2Cas9, CjeCas9, etc.), Cas12a orthologs, and other Cas3, Cas12, Cas13, and Cas14 proteins.

The Cas proteins can be incorporated into existing and widely used vectors, e.g., by simple site-directed mutagenesis, and can also be combined with other previously described improvements to the SpCas9 platform (e.g., truncated sgRNAs (Tsai et al., Nat Biotechnol 33, 187-197 (2015); Fu et al., Nat Biotechnol 32, 279-284 (2014)), nickase mutations (Mali et al., Nat Biotechnol 31, 833-838 (2013); Ran et al., Cell 154, 1380-1389 (2013)), dimeric FokI-dCas9 fusions (Guilinger et al., Nat Biotechnol 32, 577-582 (2014); Tsai et al., Nat Biotechnol 32, 569-576 (2014)); and high-fidelity variants (Kleinstiver et al. Nature 2016).

### SpCas9

The SpCas9 wild type sequence is as follows:

```
(SEQ ID NO: 64)
                10 20 30 40 50 60
MDKKYSIGLD IGTNSVGWAV ITDEYKVPSK KFKVLGNTDR HSIKKNLIGA LLFDSGETAE
                70 80 90 100 110 120
ATRLKRTARR RYTRRKNRIC YLQEIFSNEM AKVDDSFFHR LEESFLVEED KKHERHPIFG
              130 140 150 160 170 180
NIVDEVAYHE KYPTIYHLRK KLVDSTDKAD LRLIYLALAH MIKFRGHFLI EGDLNPDNSD
              190 200 210 220 230 240
VDKLFIQLVQ TYNQLFEENP INASGVDAKA ILSARLSKSR RLENLIAQLP GEKKNGLFGN
              250 260 270 280 290 300
LIALSLGLTP NFKSNFDLAE DAKLQLSKDT YDDDLDNLLA QIGDQYADLF LAAKNLSDAI
              310 320 330 340 350 360
LLSDILRVNT EITKAPLSAS MIKRYDEHHQ DLTLLKALVR QQLPEKYKEI FFDQSKNGYA
              370 380 390 400 410 420
GYIDGGASQE EFYKFIKPIL EKMDGTEELL VKLNREDLLR KQRTFDNGSI PHQIHLGELH
              430 440 450 460 470 480
AILRRQEDFY PFLKDNREKI EKILTFRIPY YVGPLARGNS RFAWMTRKSE ETITPWNFEE
              490 500 510 520 530 540
VVDKGASAQS FIERMTNFDK NLPNEKVLPK HSLLYEYFTV YNELTKVKYV TEGMRKPAFL
              550 560 570 580 590 600
SGEQKKAIVD LLFKTNRKVT VKQLKEDYFK KIECFDSVEI SGVEDRENAS LGTYHDLLKI
              610 620 630 640 650 660
IKDKDFLDNE ENEDILEDIV LTLTLFEDRE MIEERLKTYA HLFDDKVMKQ LKRRRYTGWG
              670 680 690 700 710 720
RLSRKLINGI RDKQSGKTIL DFLKSDGFAN RNFMQLIHDD SLTFKEDIQK AQVSGQGDSL
              730 740 750 760 770 780
HEHIANLAGS PAIKKGILQT VKVVDELVKV MGRHKPENIV IEMARENQTT QKGQKNSRER
              790 800 810 820 830 840
MKRIEEGIKE LGSQILKEHP VENTQLQNEK LYLYYLQNGR DMYVDQELDI NRLSDYDVDH
              850 860 870 880 890 900
IVPQSFLKDD SIDNKVLTRS DKNRGKSDNV PSEEVVKKMK NYWRQLLNAK LITQRKFDNL
              910 920 930 940 950 960
TKAERGGLSE LDKAGFIKRQ LVETRQITKH VAQILDSRMN TKYDENDKLI REVKVITLKS
              970 980 990 1000 1010 1020
KLVSDFRKDF QFYKVREINN YHHAHDAYLN AVVGTALIKK YPKLESEFVY GDYKVYDVRK
            1030 1040 1050 1060 1070 1080
MIAKSEQEIG KATAKYFFYS NIMNFFKTEI TLANGEIRKR PLIETNGETG EIVWDKGRDF
            1090 1100 1110 1120 1130 1140
ATVRKVLSMP QVNIVKKTEV QTGGFSKESI LPKRNSDKLI ARKKDWDPKK YGGFDSPTVA
            1150 1160 1170 1180 1190 1200
YSVLVVAKVE KGKSKKLKSV KELLGITIME RSSFEKNPID FLEAKGYKEV KKDLIIKLPK
            1210 1220 1230 1240 1250 1260
YSLFELENGR KRMLASAGEL QKGNELALPS KYVNFLYLAS HYEKLKGSPE DNEQKQLFVE
            1270 1280 1290 1300 1310 1320
QHKHYLDEII EQISEFSKRV ILADANLDKV LSAYNKHRDK PIREQAENII HLFTLTNLGA
            1330 1340 1350 1360
PAAFKYFDTT IDRKRYTSTK EVLDATLIHQ SITGLYETRI DLSQLGGD
```

In some embodiments, the Cas9/dCas9 has altered PAM specificity, e.g., xCas9 3.7, as described in Hu et al., Nature volume 556, pages 57-63(2018); or SpG (mutations at D1135L/S1136W/G1218K/E1219Q/R1335Q/T1337R, which targets NGN PAM sequences), or SpRY (D1135L/S1136W/G1218K/E1219Q/R1335Q/T1337R/L1111R/A1322R/A61R/N1317R/R1333P mutations, which targets almost all PAM sequences (NRN and to a lesser extent NYN PAMs) (Walton et al., Science 26 Mar. 2020:eaba8853). In some embodiments, the SpCas9 comprises a mutation at D1135E (NGG PAM); mutations at D1135V, R1335Q and T1337R (NGAN or NGNG PAM); mutations at D1135E, R1335Q and T1337R (NGAG PAM); mutations at D1135V, G1218R, R1335E and T1337R (NGCG PAM).

The SpCas9 proteins include mutations at one of the following amino acid positions to reduce or (preferably) destroy the nuclease activity of the Cas9: D10, E762, D839, H983, or D986 and H840 or N863, e.g., D10A/D10N and H840A/H840N/H840Y, to render the nuclease portion of the protein catalytically inactive; substitutions at these positions could be alanine (as they are in Nishimasu al., Cell 156, 935-949 (2014)), or other residues, e.g., glutamine, asparagine, tyrosine, serine, or aspartate, e.g., E762Q, H983N, H983Y, D986N, N863D, N863S, or N863H (see WO 2014/152432). In some embodiments, the variant includes mutations at D10A or H840A (which creates a single-strand nickase), or mutations at D10A and H840A (which abrogates nuclease activity; this mutant is known as dead Cas9 or dCas9).

The methods can include the delivery of a catalytically inactive Cas9 protein, e.g., dCas9 or dxCas9, and one or more guide RNAs that target the dCas9 to the repeat expansion region. A number of exemplary guide RNAs are provided in Table 1.

TABLE 1

| guide RNAs list | gRNA sequence | SEQ ID NO: | dCas9/dxCas9 |
|---|---|---|---|
| CGG repeat gRNA-1 | GGCGGCGGCGGCGGCGGCGG | 1. | dCas9 |
| CGG repeat gRNA-2 | GCGGCGGCGGCGGCGGCGGC | 2. | dxCas9 |
| CGG repeat gRNA-3 | CGGCGGCGGCGGCGGCGGCG | 3. | |
| CGG repeat gRNA-4 | CGCCGCCGCCGCCGCCGCCG | 4. | |
| CGG repeat gRNA-5 | GCCGCCGCCGCCGCCGCCGC | 5. | dxCas9 |
| CGG repeat gRNA-6 | CCGCCGCCGCCGCCGCCGCC | 6. | |
| FMR1-CGG-HG1 | GGGGCGTGCGGCAGCGCGG | 7. | dCas9 |
| FMR1-CGG-HG2 | GGCGTGCGGCAGCGCGGCGG | 8. | dCas9 |
| FMR1-CGG-HG3 | GTGCGGCAGCGCGGCGGCGG | 9. | dCas9 |
| FMR1-Unique-HG5 | CGCCGCTGCCAGGGGGCGTG | 10. | dCas9 |
| NegativeControl-gRNA | GGCACTGCGGCTGGAGGTGG | 11. | dCas9 |
| CAG repeat gRNA-1 | AGCAGCAGCAGCAGCAGCAG | 12. | dxCas9 |
| CAG repeat gRNA-2 | GCAGCAGCAGCAGCAGCAGC | 13. | |
| CAG repeat gRNA-3 | CAGCAGCAGCAGCAGCAGCA | 14. | |
| CTG repeat gRNA-1 | TGCTGCTGCTGCTGCTGCTG | 15. | dxCas9 |
| CTG repeat gRNA-2 | GCTGCTGCTGCTGCTGCTGC | 16. | |
| CTG repeat gRNA-3 | CTGCTGCTGCTGCTGCTGCT | 17. | |
| GAA repeat gRNA-1 | AAGAAGAAGAAGAAGAAGAA | 18. | dxCas9 |
| GAA repeat gRNA-2 | GAAGAAGAAGAAGAAGAAGA | 19. | dxCas9 |
| GAA repeat gRNA-3 | AGAAGAAGAAGAAGAAGAAG | 20. | dxCas9 |
| GAA repeat gRNA-4 | TTCTTCTTCTTCTTCTTCTT | 21. | |
| GAA repeat gRNA-5 | TCTTCTTCTTCTTCTTCTTC | 22. | |
| GAA repeat gRNA-6 | CTTCTTCTTCTTCTTCTTCT | 23. | |
| CCCCGG gRNA-1 | CCCGGCCCCGGCCCCGGCCC | 24. | dCas9 |
| CCCCGG gRNA-2 | CCGGCCCCGGCCCCGGCCCC | 25. | dxCas9 |
| CCCCGG gRNA-3 | CGGCCCCGGCCCCGGCCCCG | 26 | |
| CCCCGG gRNA-4 | GGCCCCGGCCCCGGCCCCGG | 27 | |
| CCCCGG gRNA-5 | GCCCCGGCCCCGGCCCCGGC | 28. | |
| CCCCGG gRNA-6 | CCCCGGCCCCGGCCCCGGCC | 29 | |
| CCCCGG gRNA-7 | GGGCCGGGGCCGGGGCCGGG | 30 | |
| CCCCGG gRNA-8 | GGCCGGGGCCGGGGCCGGGG | 31 | |
| CCCCGG gRNA-9 | GCCGGGGCCGGGGCCGGGGC | 32. | dCas9 |
| CCCCGG gRNA-10 | CCGGGGCCGGGGCCGGGGCC | 33. | dCas9 |
| CCCCGG gRNA-11 | CGGGGCCGGGGCCGGGGCCG | 34. | dCas9 |
| CCCCGG gRNA-12 | GGGGCCGGGGCCGGGGCCGG | 35. | dxCas9 |
| CCTG repeat gRNA-1 | CGGACGGACGGACGGACGGA | 36. | dCas9 |
| CCTG repeat gRNA-2 | GGACGGACGGACGGACGGAC | 37. | dxCas9 |
| CCTG repeat gRNA-3 | GACGGACGGACGGACGGACG | 38 | |
| CCTG repeat gRNA-4 | ACGGACGGACGGACGGACGG | 39. | |
| CCTG repeat gRNA-5 | CTGCCTGCCTGCCTGCCTGC | 40. | dxCas9 |
| CCTG repeat gRNA-6 | TGCCTGCCTGCCTGCCTGCC | 41. | dxCas9 |
| CCTG repeat gRNA-7 | GCCTGCCTGCCTGCCTGCCT | 42 | |
| CCTG repeat gRNA-8 | CCTGCCTGCCTGCCTGCCTG | 43. | |
| ATTCT repeat gRNA-1 | AGAATAGAATAGAATAGAAT | 44. | dxCas9 |
| ATTCT repeat gRNA-2 | GAATAGAATAGAATAGAATG | 45. | |
| ATTCT repeat gRNA-3 | AATAGAATAGAATAGAATGA | 46. | |
| ATTCT repeat gRNA-4 | ATAGAATAGAATAGAATGAA | 47. | |
| ATTCT repeat gRNA-5 | TAGAATAGAATAGAATGAAT | 48. | |
| ATTCT repeat gRNA-6 | ATTCTATTCTATTCTATTCT | 49. | |
| ATTCT repeat gRNA-7 | TTCTATTCTATTCTATTCTA | 50 | |
| ATTCT repeat gRNA-8 | TCTATTCTATTCTATTCTAT | 51 | |
| ATTCT repeat gRNA-9 | CTATTCTATTCTATTCTATT | 52. | |
| ATTCT repeat gRNA-10 | TATTCTATTCTATTCTATTC | 53. | |
| TGGAA repeat gRNA-1 | TGGAATGGAATGGAATGGAA | 54. | dCas9 |
| TGGAA repeat gRNA-2 | GGAATGGAATGGAATGGAAT | 55. | dxCas9 |
| TGGAA repeat gRNA-3 | GAATGGAATGGAATGGAATG | 56. | dxCas9 |
| TGGAA repeat gRNA-4 | AATGGAATGGAATGGAATGG | 57. | |
| TGGAA repeat gRNA-5 | ATGGAATGGAATGGAATGGA | 58. | |
| TGGAA repeat gRNA-6 | TTCCATTCCATTCCATTCCA | 59. | |
| TGGAA repeat gRNA-7 | TCCATTCCATTCCATTCCAT | 60. | |
| TGGAA repeat gRNA-8 | CCATTCCATTCCATTCCATT | 61. | |
| TGGAA repeat gRNA-9 | CATTCCATTCCATTCCATTC | 62. | |
| TGGAA repeat gRNA-10 | ATTCCATTCCATTCCATTCC | 63. | |

### Delivery and Expression Systems

The methods can include delivering the dCas9 in a nucleic acid that encodes them. This can be performed in a variety of ways. For example, the nucleic acid encoding the dCas9 can be delivered as mRNA, or can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the dCas9 for production of the dCas9. The nucleic acid encoding the dCas9 can also be cloned into an expression vector, for administration to a plant cell, animal cell, preferably a mammalian cell or a human cell, fungal cell, bacterial cell, or protozoan cell.

To obtain expression, a sequence encoding a dCas9 is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In contrast, when the dCas9 is to be administered in vivo for gene regulation, either a constitutive or an inducible promoter can be used, depending on the particular use of the dCas9. In addition, a preferred promoter for administration of the dCas9 can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88: 1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the dCas9, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the dCas9, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

Expression vectors containing regulatory elements from eukaryotic viruses are often used in eukaryotic expression vectors, e.g., lentiviral vectors, adenoviral vectors, SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the dCas9s can include RNA Pol III promoters to drive expression of the guide RNAs, e.g., the H1, U6 or 7SK promoters. These human promoters allow for expression of dCas9s in mammalian cells following plasmid transfection.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the Cas9.

Alternatively, the methods can include delivering the dCas9 protein and guide RNA together, e.g., as a complex. For example, the dCas9 and gRNA can be overexpressed in a host cell and purified, then complexed with the guide RNA (e.g., in a test tube) to form a ribonucleoprotein (RNP), and delivered to cells. In some embodiments, the variant dCas9 can be expressed in and purified from bacteria through the use of bacterial dCas9 expression plasmids. For example, His-tagged variant dCas9 proteins can be expressed in bacterial cells and then purified using nickel affinity chromatography. The use of RNPs circumvents the necessity of delivering plasmid DNAs encoding the nuclease or the guide, or encoding the nuclease as an mRNA. RNP delivery may also improve specificity, presumably because the half-life of the RNP is shorter and there's no persistent expression of the nuclease and guide (as you'd get from a plasmid). The RNPs can be delivered to the cells in vivo or in vitro, e.g., using lipid-mediated transfection or electroporation. See, e.g., Liang et al. “Rapid and highly efficient mammalian cell engineering via Cas9 protein transfection.” Journal of biotechnology 208 (2015): 44-53; Zuris, John A., et al. “Cationic lipid-mediated delivery of proteins enables efficient protein-based genome editing in vitro and in vivo.” Nature biotechnology 33.1 (2015): 73-80; Kim et al. “Highly efficient RNA-guided genome editing in human cells via delivery of purified Cas9 ribonucleoproteins.” Genome research 24.6 (2014): 1012-1019.

### Methods of Use

In some embodiments, the present methods and compositions can be used to contract nucleotide expansions in a living cell or population of cells from a subject with a condition associated with nucleotide expansions, e.g., a number of nucleotide repeats above a reference number, where the reference number represents a number in a normal individual or in a subject who is not affected; in some embodiments the reference number represents a range of numbers of repeats present in a person who has a permutation or is a carrier of the disease but is unaffected. A non-limiting list of conditions includes FXS, fragile X-associated tremor/ataxia syndrome (FXTAS), Parkinson's disease (PD), Huntington's disease (HD), myotonic dystrophy, and others. A non-limiting list of nucleotide repeats includes CGG repeats, GGC repeats, CAG repeats, CTG repeats, and GAA repeats. Repeats of more than three nucleotides can also be targeted as nucleotide repeats are defined as sequences of 1-6 nucleotides (or longer, e.g., 10 nts) that are repeated multiple times. See, e.g., Budworth, H. and McMurray, C. T. (2013) Methods Mol Biol. 1010:3-17; Ellerby (2019) Neurotherapeutics 16:924-927. Table 2 provides a list of exemplary repeats and associated diseases.

TABLE 2
Repeat expansion diseases

| | | | | |
|---|---|---|---|---|
| | | | Normal | Symptomatic |
| Disorder | Affected gene | Repeat | repeat no | repeat no. |

Repeats in coding regions

| | | | | |
|---|---|---|---|---|
| DRPLA | ATNI | CAG | 7-25 | 49-88 |
| HD | HTT | CAG | 6-34 | 36-180 |
| HDL2 | JPH3 | CAG or CTG | 6-28 | 40-58 |
| PD/NIID | NOTCH2NLC | GGC | 5-39 | 40-517 |
| SBMA | AR | CAG | 11-24 | 40-62 |
| SCA1 | ATXNI | CAG | 6-39 | 39-83 |
| SCA2 | ATXN2 | CAG | 15-29 | 34-59 |
| SCA3 | ATXN3 | CAG | 13-36 | 55-84 |
| SCA6 | CACNAIA | CAG | 4-16 | 21-30 |
| SCA7 | ATXN7 | CAG | 4-35 | 34→300 |
| SCA8 | ATXN8 | CAG | <80 | 80-250 |
| SCA17 | TBP | CAG | 25-44 | 45-66 |
| XLMR | ARX | GCG | <20 | 20-23 |

Repeats in Noncoding loci (including 5′ UTR, 3′ UTR and intron)

| | | | | |
|---|---|---|---|---|
| DM1 | DMPK | CTG | 5-37 | >50→2000 |
| DM2 | CNBP | CCTG | <27 | 75-11,000 |
| | | (SEQ ID NO: 65) | | |
| EPM1 | CSTB | (C)₄G(C)₄GCG | 2-3 | 30-75 |
| | | (SEQ ID NO: 66) | | |
| FTD/ALS | C9ORF72 | GGGGCC | 2-23 | 700-1600 |
| | | (SEQ ID NO: 67) | | |
| | | or CCCCGG | | |
| | | (SEQ ID NO: 68) | | |
| FXS/FXTAS | FMRI | CGG | 6-50 or | 55->2000 |
| | | | 52 | |
| FRAXE MR | AFF2/FMR3 | CCG | 6-25 | >200 |
| FRA12A MR | DIP28 | CGG | 6-23 | >200 |
| FRDA | FXN | GAA | 7-22 | >66->900 |
| SCA10 | ATXN10 | ATTCT | 10-29 | 280-4500 |
| | | (SEQ ID NO: 69) | | |
| SCA31 | ATAXN31 | TGGAA | <110 | > = 110 |
| | | (SEQ ID NO: 70) | | |
| XDP | TAF1 | CCCTCT | <35 | > = 35 |
| | | (SEQ ID NO: 71) | | |

Repeats in coding or noncoding loci

| | | | | |
|---|---|---|---|---|
| SCA8 | ATXNB/ATXNBOS | CAG and CTG | 6-37 | ~107-250 |

Repeats with uncertain loci

| | | | | |
|---|---|---|---|---|
| SCA12 | PPP2R28 | CAG or CTG | <66 | > = 66 |

(DM1) myotonic dystrophy type 1; (DM2) myotonic dystrophy type 2; (DRPLA) dentatorubral-pallidoluysian atrophy; (EPM1) progressive myoclonus epilepsy type I; (FTD/ALS) Frontotemporal dementia (FTD) and amyo-trophic lateral sclerosis (ALS); (FXS) Fragile X syndrome; (MR) mental retardation; (FRDA) Friedreich ataxia; (HD) Huntington disease; (HDL-2) Huntington disease-like 2; (NCT) noncoding transcript; (SBMA) spinobulbar muscular atrophy; (SCA) spinocerebellar ataxia. Table adapted in part from Usdin, K (2008) Genome Res. 18: 1011-1019.

The methods can include obtaining iPSC generated from differentiated somatic cells obtained from the subject; exposing the iPSC to a treatment described herein to contract (reduce the number of) nucleotide repeats; optionally promoting differentiation of the corrected cells, e.g., to neural precursor cells; and administering the cells to the subject, e.g., to the CNS (spinal cord or brain) of a subject, such as to the cortex, cerebellum, hypothalamus, substantia nigra, spinal cord, putamen, hippocampus, or other CNS regions (see, e.g., Duma et al., Molecular Biology Reports volume 46, pages 5257-5272(2019); Schweitzer et al., N Engl J Med. 2020 May 14; 382(20):1926-1932; Kim et al., Alzheimers Dement (N Y). 2015 September; 1(2): 95-102), or to one or more organs (e.g., liver, lung, heart, kidney, or gut). Alternatively, the methods can include administering a composition as described herein to the subject, e.g., to the CNS of the subject (e.g., via ICV, cisternae magna, or intrathecal administration), to an organ (e.g., liver, lung, heart, kidney, or gut) or systemically. A few exemplary conditions are discussed in greater detail in the following paragraphs.

### Methods of Treating FXS

The present methods and compositions can be used to treat subjects who have fragile X syndrome (FXS). A diagnosis of FXS can be made using methods known in the art. For example, a standard diagnostic test for FXS involves using molecular genetic techniques to detect and/or sequence the FMR1 gene; in some cases the exact number of CGG triplet repeats can be determined. Methods for diagnosing FXS include Southern blotting and polymerase chain reaction (PCR) (see, e.g., Stone W L, Basit H, Los E. Fragile X Syndrome. [Updated 2020 May 4]. In: StatPearls [Internet]. Treasure Island (FL): StatPearls Publishing; 2020 January Available from ncbi.nlm.nih.gov/books/NBK459243/). Normal numbers of CGG repeats are between five and 40 repeats. Individuals with 55 to 200 repeats are considered to have an FMR1 gene premutation (PM) and typically have a normal intellect. Individuals with greater than 200 CGG repeats have a full mutation (FM) for FXS. In some embodiments, the methods and compositions described herein can be administered to a cell or subject having >50 repeats, e.g., 50-200 repeats, or >200 repeats. In subjects with 50-200, the methods can be used for contracting CGG repeats, e.g., for the purposes of reducing CGG-FMR1 accumulation in the brains of Fragile X premutation carriers.

Fragile X syndrome, also termed Martin-Bell syndrome or marker X syndrome, is one of the most common heritable neurodegenerative disorders and is caused by the expansion of CGG nucleotide repeats in the 5′-UTR of the FMR1 gene. There are two types of FXS with distinct pathological lesions depending on the length of the CGG repeats: pre-mutation and full mutation. Very long expansion resulting in over 200 CGG repeats—so-called full mutation (FM)—cause DNA hyper-methylation and consequent epigenetic silencing of the fragile X mental retardation 1 (FMR1) gene, which in turn causes a loss of expression of the fragile X mental retardation protein (FMRP) (McLennan et al., Curr Genomics. 2011 May; 12(3): 216-224). FMRP is a polyribosome-associated RNA binding protein that regulates the translation of mRNAs from a wide variety of genes, including many genes encoding synaptic proteins (Darnell, J. C., et al., Cell, 2011. 146(2): p. 247-61). Currently, there is no cure for FXS or FXTAS Parkinsonism available, and treatments are limited to alleviating symptoms.

While typically apparently normal at birth, signs of FXS can develop in early childhood, signaled by developmental delays, psychomotor delays, intellectual disabilities, elongated face, prominent ears, and post-pubertal macroorchidism (McLennan Y, et al., Curr Genomics. 2011 May; 12(3):216-24). Otitis media and sinusitis can be frequent, leading to conductive hearing problems and further developmental delays. Approximately 30% to 60% of individuals with FXS have autism. Obesity is a common problem in subjects with FXS.

The present methods and compositions can be used to treat subjects who have FXS. In some embodiments, the subject has demonstrated signs of FXS; in some embodiments, the subject has not yet demonstrated signs of FXS. The methods can thus be used to ameliorate one or more symptoms of FXS, e.g., to reduce severity of one or more symptoms; to reduce the likelihood that a subject will develop one or more symptoms of FXS; or to slow progression or worsening of one or more symptoms of FXS.

### Methods of Treating FXTAS

The present methods and compositions can be used to treat subjects who have fragile X-associated tremor/ataxia syndrome (FXTAS). A diagnosis of FXTAS can be made using methods known in the art. Some signs and symptoms of FXTAS include, but are not limited to, parkinsonism (resting tremors), intention tremors, ataxias, gait disturbances, MRI findings (white matter lesions involving middle cerebellar peduncles (MCP) signs, moderate to severe generalized brain atrophy, etc.), and neuropathology signs (e.g., inclusions with brain cells), short-term memory problems, and defects in “executive function” and decision-making.

The cause of FXTAS is PM (pre-mutation; discussed above), where subjects typically have 50-200 repeats of CGG. Carriers of the pre-mutation alleles (55-200 CGG repeats) exhibit increased FMR1 mRNA levels but normal or lower FMRP expression, implying a different pathological mechanism (Tassone, F., et al., RNA, 2007. 13(4): p. 555-62). Such carriers are asymptomatic earlier in life but develop Parkinsonism later in life—a condition called FXTAS and presumed to be caused by toxic accumulation of CGG-FMR1 RNA (Ma, L, et al., (2019) Acta Neuropathologica Communications 7:143; Kraff J, et al., (2007) Arch Neurol 64(7):1002-1006). As discussed above, individuals with PM have 50-200 CGG repeats (e.g., in FMR1 or NOTCH2NLC). FXTAS affects 40% of PM male carries and 15% of female PM carries. Generally, there is a high prevalence of premutation, which is 1 in 259 for men and 1 in 813 for women. Premutation causes FXTAS and is one of the most common causes of age-related neurodegenerative disorders (e.g., ALS, PD, etc). In some embodiments, the methods and compositions described herein can be administered to a cell or subject having >50 repeats, 50-200 repeats, or >200 repeats.

The present methods and compositions can be used to treat subjects who have FXTAS. In some embodiments, the subject has demonstrated signs of FXTAS; in some embodiments, the subject has not yet demonstrated signs of FXTAS. The methods can thus be used to ameliorate one or more symptoms of FXTAS, e.g., to reduce severity of one or more symptoms; to reduce the likelihood that a subject will develop one or more symptoms of FXTAS; or to slow progression or worsening of one or more symptoms of FXTAS.

### Methods of Treating PD

The present methods and compositions can be used to treat subjects who have Parkinson's disease (PD) associated with nucleotide repeat expansion. A diagnosis of PD with TNR expansion can be made using methods known in the art. The presence of Notch homolog 2 N-terminal-like C (NOTCH2NLC) GGC (or CGG) expansion causes neuronal intranuclear inclusion body disease (NIID), a neurodegenerative condition characterized by eosinophilic intranuclear inclusions in neuronal and glial cells. Large GGC repeat numbers, ranging from 66 to 517 units, have been reported in patients affected by NIID, while control participants had fewer than 40 repeats (5-39). An intermediate-length (40-65) NOTCH2NLC GGC expansion may also occur in human neurological disease (Shi et al., Ann Neurol 2021; 89:182-187). Patients with NIID have overlapping clinical features of dementia, ataxia, parkinsonism, and peripheral neuropathy. While parkinsonism is part of the spectrum of the clinical phenotype and may be a predominant sign in some patients with NIID and a family history, subjects with NOTCH2NLC GGC repeat expansions can present with typical sporadic PD with no other clinical or imaging features of NIID, even after several years of follow-up. The higher frequency of GGC expansions of 41 to 64 repeats in patients with PD compared with controls (Ma, D., et al. (2020) JAMA Neurol. 2020 Aug. 24; 77(12):1-5). GGC expansion in NOTCH2NLC is also associated with adult leukoencephalopathy (Okubo et al., Ann Neurol. 2019 December; 86(6):809-811).

The cause of PD is typically a mix of environmental and genetic causes. However, nucleotide repeat expansion has been shown in subjects with PD. As discussed above, some individuals with PD have 40-517 CGG repeats in genes associated with PD (e.g., NOTCH2NLC). In some embodiments, the methods and compositions described herein can be administered to a cell or subject having >40 repeats, e.g., 40-200 repeats, or >200 repeats.

The present methods and compositions can be used to treat subjects who have PD or NIID. In some embodiments, the subject has demonstrated signs of PD; in some embodiments, the subject has not yet demonstrated signs of PD. The methods can thus be used to ameliorate one or more symptoms of PD, e.g., to reduce severity of one or more symptoms; to reduce the likelihood that a subject will develop one or more symptoms of PD; or to slow progression or worsening of one or more symptoms of PD.

### Methods of Treating HD

The present methods and compositions can be used to treat subjects who have Huntington's disease (HD). A diagnosis of HD can be made using methods known in the art. The cause of Huntington's disease was found to be a CAG expansion in exon 1 of the huntingtin gene (HTT). The disease protein contains a polyglutamine expansion in the N-terminal region of the Huntingtin protein (HTT) (Ellerby, L. M. (2019) Neurotherapeutics 16:924-927). Unaffected individuals may have roughly 6-29 CAG triplets in both alleles; yet, in HD patients, the disease allele may contain 36 to hundreds of CAG triplets. As the repeat number grows, the growing polyglutamine tract produces an abnormal HD gene product (called huntingtin) with increasingly aberrant properties that causes death of brain cells controlling movement (Budworth, H. and McMurray, C. T. (2013) Methods Mol Biol. 1010:3-17). In some embodiments, the methods and compositions described herein can be administered to a cell or subject having >30 repeats, e.g., 30-100 repeats, or >100 repeats. In some embodiments, the methods and compositions described herein methods can reduce levels of huntingtin.

The present methods and compositions can be used to treat subjects who have HD. In some embodiments, the subject has demonstrated signs of HD; in some embodiments, the subject has not yet demonstrated signs of HD. The methods can thus be used to ameliorate one or more symptoms of HD, e.g., to reduce severity of one or more symptoms; to reduce the likelihood that a subject will develop one or more symptoms of HD; or to slow progression or worsening of one or more symptoms of HD.

### Methods of Treating Myotonic Dystrophy

The present methods and compositions can be used to treat subjects who have Myotonic dystrophy, or dystrophia myotonica (DM), including myotonic dystrophy type 1 (DM1) and/or myotonic dystrophy type 2 (DM2). A diagnosis of DM1 and/or DM2 can be made using methods known in the art. Myotonic dystrophy exists in two clinically and molecularly defined forms: myotonic dystrophy type 1 (DM1), also known as Steinert's disease; and myotonic dystrophy type 2 (DM2), also known as proximal myotonic myopathy, both of which are inherited in an autosomal dominant fashion [9]. DM1 is caused by a CTG expansion in the 3′ untranslated region of the dystrophia myotonica protein kinase (DMPK) gene on chromosome 19q13, while DM2 is caused by a CCTG expansion located within intron 1 of the cellular nucleic-acid-binding protein (CNBP, formerly ZNF9) gene on chromosome 3q21 (Yum K, et al. (2017) Curr Opin Genet Dev. 44: 30-37).

A healthy individual with normal DMPK alleles has 5 to 37 repeats (35 has also commonly been used as an upper threshold for normal repeat length). DM1 patients who have repeats between 38 and 50 are said to have a “pre-mutation” allele and can be asymptomatic throughout their lifetime. However, they are at increased risk of having children with larger repeats. In DM1, there can be hundreds or even thousands of CTG repeats in the DMPK gene. In DM1, the number of repeats correlates with the age of onset and the severity of the disorder. However, in DM2 there is no definite correlation between repeat length and the severity of disease. It is important to remember that these correlations are by no means perfect and should not be taken as absolute predictors of the course of the disease.

Individuals with a CTG repeat size between 38 and 49, designated premutation status or mutable normal, are asymptomatic. A mutation of 50 to approximately 150 CTG repeats can manifest as a mild DM1 type. Repeats in the range of 50 to 1,000 are seen in individuals with classic DM1. CTG repeat lengths greater than 800 may manifest as childhood DM1. With CTG repeat lengths greater than 1,000, DM1 may manifest as congenital MD. A phenomenon known as somatic mosaicism was observed in DM1 patients. This phenomenon results in expansion of CTG repeats in the DNA due to abnormal DNA repair throughout life. When the DMPK gene expansion is transmitted from parent to child, it often expands, causing the disease to manifest earlier with each generation in a family.

Penetrance tends to grow as repeat length increases, but extreme variability in penetrance of specific symptoms exists in the patient population. Somatic mosaicism and intergenerational instability are biased towards expansion in DM1, although contraction can rarely occur. It is estimated that a decrease in the CTG repeat size during transmission from parents to child is about 6.4%, most frequently during paternal transmissions. Children of DM1 parents typically inherit repeat lengths considerably larger than those present in the transmitting parent, the phenomenon known as “anticipation,” where disease severity increases and age of onset decreases in successive generations. Up to 5% of DM1 patients have interrupted repeats, in which the CTG repeat tract contains GGC, CCG, or CTC repeats. Some of these interruptions have been associated with stabilization of the CTG repeat tract length.

The repeat expansion of DM2 in intron 1 of CNBP is found within the context of a complex (TG)n(TCTG)n(CCTG)n sequence or CCTG—repeated far more times than average. The normal gene has 11 to 26 repeats; on genes of those with DM2, there are from 75 to more than 11,000 repeats, with a mean of 5,000 repeats. CCTG repeat tracts also display somatic instability. While non-pathogenic alleles contain up to 26 repeats, the range of repeats in patients is extremely broad, with measurements from 75 to 11,000 units (on average 5,000). Unlike DM1, the size of the repeat DNA expansion in DM2 does not correlate with age of onset or disease severity. This is further supported by the observation that individuals homozygous for repeat expansions have clinical features indistinguishable from that of their heterozygous siblings. Phenotypes and anticipation in DM2 are almost always milder than DM1, and DM2 lacks the congenital form. Despite these key differences, DM1 and DM2 share several hallmark clinical features such as myotonia, cataracts, and cardiac conduction defects (Yum K, et al. (2017) Curr Opin Genet Dev. 44: 30-37).

In some embodiments, the methods and compositions described herein can be administered to a cell or subject having >25 repeats, e.g., 25-100 repeats, >35 repeats, e.g., 35-100 repeats, >50 repeats, e.g., 50-150 repeats, or >100 repeats.

The present methods and compositions can be used to treat subjects who have DM1 and/or DM2. In some embodiments, the subject has demonstrated signs of DM1 and/or DM2; in some embodiments, the subject has not yet demonstrated signs of DM1 and/or DM2. The methods can thus be used to ameliorate one or more symptoms of DM1 and/or DM2, e.g., to reduce severity of one or more symptoms; to reduce the likelihood that a subject will develop one or more symptoms of DM1 and/or DM2; or to slow progression or worsening of one or more symptoms of DM1 and/or DM2.

### Induced Pluripotent Stem Cells (iPSC)

The methods described herein can include the use of human induced pluripotent stem cells (hiPSCs), which can be generated using methods known in the art or described herein. In some embodiments, the methods for generating hiPSC can include obtaining a population of primary somatic cells from a subject who has been diagnosed with a disease associated with nucleotide repeats, e.g., FXS, and is in need of treatment for the disease. Preferably the subject is a mammal, e.g., a human. In some embodiments, the somatic cells are fibroblasts. Fibroblasts can be obtained from connective tissue in the mammalian body, e.g., from the skin, e.g., skin from the eyelid, back of the ear, a scar (e.g., an abdominal cesarean scar), or the groin (see, e.g., Fernandes et al., Cytotechnology. 2016 March; 68(2): 223-228), e.g., using known biopsy methods. Other sources of somatic cells for hiPSC include hair keratinocytes (Raab et al., Stem Cells Int. 2014; 2014:768391), blood cells, or bone marrow mesenchymal stem cells (MSCs) (Streckfuss-Bömeke et al., Eur Heart J. 2013 September; 34(33):2618-29).

According to the present methods, the cells (e.g., fibroblasts) are exposed to factors to induce reprogramming to iPSC. Although other protocols for programming can be used (e.g., as known in the art or described herein), in preferred embodiments the methods include introducing four transcription factors, i.e., Oct4, Sox2, Klf4, and L-Myc. In some embodiments, the methods comprise transfecting the cells with an OCT4, KLF4, SOX2, and L-MYC-expressing polycistronic episomal vector. See, e.g., WO 2020/237104.

References to exemplary sequences for OCT4, KLF4, SOX2, and L-MYC are provided in table 3.

TABLE 3
Sequences for some transcription factors.

| Gene | Nucleic acid → protein | |
|---|---|---|
| OCT4 | NM_002701.6 → NP_002692.2 | Isoform 1 |
| (POU class 5 | NM_001173531.2 → NP_001167002.1 | Isoform 2 |
| homeobox 1 | NM_001285987.1 → NP_001272916.1 | Isoform 3 |
| (POU5F1)) | NM_001285986.1 → NP_001272915.1 | Isoform 4 |
| KLF4 | NM_001314052.1 → NP_001300981.1 | Isoform 1 |
| (Kruppel like | NM_004235.6 → NP_004226.3 | Isoform 2 |
| factor 4) | | |
| SOX2 | NM_003106.4 → NP_003097.1 | |
| (SRY-box 2) | | |
| L-MYC | NM_001033081.3 → NP_001028253.1 | Isoform 1 |
| (MYCL proto- | NM_005376.4 → NP_005367.2 | Isoform 2 |
| oncogene, bHLH | NM_001033082.2 → NP_001028254.2 | Isoform 3 |
| transcription | | |
| factor) | | |

In some embodiments, the methods also or alternatively include expressing in the cells one or more exogenous microRNAs, e.g., one or more of miR-106a, -106b, -136s, -200c, -302s, -369s, and -371/373. miR-302s indicates the miR-302 cluster which encompasses five miRNAs including 302a, 302b, 302c, 302d, and 367; any one or more of them can be used. In some embodiments, the methods include expressing in the cells miR-302s and miR-200c, e.g., from a single episomal vector. In some embodiments, the methods comprise introducing into the cells an episomal vector that comprises sequences coding for miR-302s and miR-200c. See, e.g., WO 2020/237104.

The sequences used can be at least 80, 85, 90, 95, or 100% identical to the exemplary (reference) sequences provided herein, but should retain the desired activity of the exemplary (reference) sequence. Calculations of “identity” between two sequences can be performed as follows. The sequences are aligned for optimal comparison purposes (e.g., gaps can be introduced in one or both of a first and a second nucleic acid sequence for optimal alignment and non-identical sequences can be disregarded for comparison purposes). The length of a sequence aligned for comparison purposes is at least 60% (e.g., at least 70%, 80%, 90% or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two nucleotide sequences is determined using the GAP program in the GCG software package, using a BLOSUM 62 scoring matrix with a gap penalty of 12, a gap extend penalty of 4, and a frameshift gap penalty of 5.

The primary somatic cells can be transfected directly, or they can be cultured first, removed from the culture plate and resuspended before transfection is carried out. The cells are combined with exogenous nucleic acid sequence, e.g., to—express the reprogramming factors by transient transfection or to stably integrate into the genomes. As used herein, the term “transfection” includes a variety of techniques for introducing an exogenous nucleic acid into a cell including calcium phosphate or calcium chloride precipitation, microinjection, DEAE-dextrin-mediated transfection, lipofection, or electroporation, all of which are known in the art. Where the vectors are viral vectors, transfection can include transducing the cells with viral particles. For episomal vector delivery, cells can be electroporated with reprogramming factors-expressing episomal vectors (e.g. using a commercially available or known method, e.g., AMAXA or NEON transfection system; Song et al., (2020) J Clin Invest. 130(2): 904-920; Meggis et al., (2016) Stem Cell Research 16: 128-132). Alternatively, the cells can be transfected with mRNA encoding each of the factors.

After introducing these factors into the cells, the cells are maintained in conditions and for a time sufficient for expression of the factors and induction of reprogramming to iPS cells, e.g., cells that express alkaline phosphatase (AP) as well as the more stringent pluripotency marker, TRA-1-60 (Chan et al., 2009; Tanabe et al., 2013). A number of methods are known in the art; see, e.g., Malik and Rao, Methods Mol Biol. 2013; 997:23-33. In some embodiments, the conditions comprise maintaining the cells in media, e.g., media comprising DMEM/F-12, L-glutamine (e.g., 2 mM), serum, e.g., fetal bovine serum (FBS) (e.g., 10%), Non-essential amino acid (NEAA, e.g., 1×), Nicotinamide (NAM, e.g., 1 mM), Sodium butyrate (NaB) (e.g., 25 mM), and Ascorbic acid (AA, e.g., 50 μg/ml); alternatively, DMEM media with knockout serum replacement (KSR, a defined, FBS-free medium), glutamine, and β-mercaptoethanol can be used. One of skill in the art will appreciate that other concentrations can be used. In some embodiments, the conditions comprise maintaining the cells in media with feeder cells, which are non-dividing supporter cells (e.g., irradiated embryonic fibroblasts, irradiated mouse embryonic fibroblasts). Methods can also be found the following references: Yu G., et al., (2015) “Feeder Cell Sources and Feeder-Free Methods for Human iPS Cell Culture.” In: Sasaki K., Suzuki O., Takahashi N. (eds) Interface Oral Health Science 2014. Springer, Tokyo. (pp. 145-159; doi.org/10.1007/978-4-431-55192-8_12); Thomson J A, et al. (1998) Embryonic stem cell lines derived from human blastocysts. Science 282(5391): 1145-7; Hoffman L M, Carpenter M K. Characterization and culture of human embryonic stem cells. Nat Biotechnol. 2005; 23(6):699-708; Pera M F, Reubinoff B, Trounson A. Human embryonic stem cells. J Cell Sci. 2000; 113(1):5-10, for example.

Following reprogramming to iPSC, the cells can be maintained in an hiPSC medium, e.g., comprising DMEM/F-12, L-glutamine (e.g., 2 mM), KSR (e.g., 20%), NEAA, NAM, NaB, and bFGF with feeder cells, until formation of ES-like colonies. Once established, the pluripotency of hiPSCs can be further confirmed by differentiating into three germ layers (mesoderm, ectoderm, and endoderm) and test their identities by (1) staining with antibodies against the three germ layer markers (OTX2, an ectodermal marker; SOX17, an endodermal marker; and BRACHYURY, a mesodermal marker) and (2) gene expression of lineage-specific markers (e.g., PAX6 and MAP2 for ectoderm, FOXA2, SOX17 and CK8 for endoderm, and MSX1, MYL2A and COL6A2 for mesoderm). In some embodiments for serum-free and feeder-free conditions, the iPSC cells are maintained in ESSENTIAL 8 medium or an equivalent thereof, i.e., comprising or consisting essentially of DMEM F-12, L-ascorbic acid, Selenium, Transferrin, NaHCO₃, Insulin, FGF2, and TGFβ1. See, e.g., Chen et al., Nat Methods 8(5):424-429. In some embodiments, the iPSC cells are maintained in mTeSR media (from StemCell Technology) designed for serum-free and feeder-free conditions. See, e.g., Shi, M.-J., Stencel, K. and Borowski, M. (2010). “Human Embryonic Stem Cell Culture on BD Matrigel™ with mTeSR®1 Medium.” In Human Stem Cell Technology and Biology (eds G. S. Stein, M. Borowski, M. X. Luong, M.-J. Shi, K. P. Smith and P. Vazquez). https://doi.org/10.1002/9780470889909.ch11. Methods of reprogramming and maintaining iPSCs can also be found in the art; see, e.g., Ghaedi, M. and Niklason, L. E. (2019) Methods Mol Biol. 1576: 55-92; and Takahashi K, et al. (2007) Cell 131(5):861-72.

Once iPS cells are generated, they can be maintained as an iPS cell line. In some embodiments, for each patient, multiple iPSC lines are generated and characterized and then the best lines (e.g., the best 1, 2, 3 or more lines) are chosen.

### Methods for Contracting Nucleotide Repeats, Reactivating Silent Genes, and Restoring Function of Disease-Related Genes

Once iPSC are obtained or generated from a subject with a disease associated with nucleotide repeats, e.g., FXS, the cells or cell lines can be subjected to treatment with a combination of dCas9 and one or more guide RNAs (e.g., introduced by viral, e.g., lentiviral or adenoviral vectors) that leads to contraction of nucleotide repeats and subsequent alleviation of the abnormality caused by the expanded repeats, e.g., restoring the function of FMR1 gene by the contraction of CGG repeats.

The cells are provided with dCas9 and gRNAs for a time sufficient for the cells to restore the gene function by contraction of nucleotide repeats to occur, e.g., at least 2, 3, 4, 5, 6, 7, 8, 9, 10, 11, 12, 13, 14, 15, 16, 17, 18, 19, 20, 21, 22, 23, 24, 25, 26, 27, or 28 days or longer. The cells can be provided with dCas9 and gRNAs via transfection methods known in the art and discussed elsewhere herein. For example, cells can be combined with exogenous nucleic acid sequence, e.g., to transiently express the reprogramming factors by transient transfection or to stably integrate into the genomes. Where the vectors are used viral vectors (e.g., lentiviral vector), transfection can include transducing the cells with viral particles (e.g., lentivirus). For episomal vector delivery, cells can be, e.g., electroporated, with reprogramming factors-expressing episomal vectors (e.g. using a commercially available or known method, e.g., AMAXA or NEON transfection system; Song et al., (2020) J Clin Invest. 130(2): 904-920; Meggis et al., (2016) Stem Cell Research 16: 128-132). In some embodiments, an RNP (RNA-Protein complex) can be used to deliver the dCas9 and gRNA to the cells, which can be repeated every 2-3 days.

The methods can include generating new clonal cell lines, e.g., from single cells, and determining the presence of restored disease-related gene (e.g. FMR1) function and reduced numbers of CGG repeats, and selecting clonal cell lines with restored gene function and reduced numbers of CGG repeats (e.g., fewer than 200, 100, 75, 80, or 50 CGG repeats). The presence of other spontaneous mutations can also be determined, e.g., using next generation sequencing to identify cells with oncogenic or other deleterious mutations. Once these cells with restored FMR1 gene function are generated, they can be maintained as an iPS cell line. In some embodiments, for each patient, multiple iPSC lines with restored FMR1 gene function are generated and characterized and then the best lines (e.g., the best 1, 2, 3 or more lines) are chosen.

The cells can be cultured in the presence of a catalytically inactive Cas9 protein, e.g., dCas9 or a variant thereof, and one or more guide RNAs that target the dCas9 to the repeat expansion region. A number of exemplary guide RNAs are provided in Table 1, above.

In some embodiments, the cells are maintained in the reactivation cocktail for a time sufficient for the cells to reactivate gene expression and for contraction of nucleotide repeats to occur, e.g., at least 2, 3, 4, 5, 6, 7, 8, 9, 10, 11, 12, 13, 14, 15, 16, 17, 18, 19, 20, 21, 22, 23, 24, 25, 26, 27, or 28 days or longer. The methods can include generating new clonal cell lines, e.g., from single cells, and determining the presence of restored disease-related gene (e.g. FMR1) function, increased gene expression (e.g., of FMR1) and/or reduced numbers of repeats, and selecting clonal cell lines with increased gene expression (e.g., of FMR1) and reduced numbers of nucleotide repeats (e.g., CGG repeats (e.g., fewer than 200, 100, 75, 80, or 50 CGG repeats)). The presence of other spontaneous mutations can also be determined, e.g., using next generation sequencing to identify cells with oncogenic or other deleterious mutations. Once these corrected, e.g., FMR1 gene-reactivated, cells are generated, they can be maintained as an iPS cell line. In some embodiments, for each patient, multiple (e.g., FMR1-reactivated) iPSC lines are generated and characterized and then the best lines (e.g., the best 1, 2, 3 or more lines) are chosen.

The cells can be cultured in the presence of a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor;
(v) a MEK inhibitor, a Raf inhibitor, a ROCK1 inhibitor, a GSK-3β inhibitor, and a Src Inhibitor. In some embodiments, the factors do not include a Src Inhibitor and/or a GSK-3β inhibitor. In some embodiments, the factors do not include a Src Inhibitor and either a GSK-3β inhibitor or a ROCK1 inhibitor. In some embodiments, the Src inhibitor is a dual Lck/Src Inhibitor.
   MEK inhibitors

A number of MEK inhibitors (which specifically inhibit mitogen-activated protein kinase kinase enzymes MEK1 and/or MEK2) are known in the art. For example, Trametinib (GSK1120212) has been used for the treatment of certain cancers. Other examples of MEK inhibitors include Selumetinib, MEK162, PD-325901, cobimetinib (XL518; [3,4-Difluoro-2-(2-fluoro-4-iodoanilino)phenyl] {3-hydroxy-3-[(2S)-piperidin-2-yl]azetidin-1-yl} methanone), CL-1040, and PD035901.

### ROCK1 2 Inhibitors

A number of small molecule inhibitors of ROCK1/2 are known in the art and can be used in the present methods and compositions including cyclohexanecarboxamides such as Y-27632 ((+)-(R)-trans-4-(1-aminoethyl)-N-(4-pyridyl)cyclohexanecarboxamide dihydrochloride) and Y-30131 ((+)-(R)-trans-4-(1-aminoethyl)-N-(1H-pyrrolo[2,3-b]pyridin-4-yl)cyclohexanecarboxamide dihydrochloride)(see Ishizaki et al., Mol Pharmacol. 2000 May; 57(5):976-83); dihydropyrimidinones and dihydropyrimidines, e.g., bicyclic dihydropyrimidine-carboxamides (such as those described in Sehon et al. J. Med. Chem., 2008, 51 (21): 6631-6634 and US2018/0170939); ureidobenzamides such as CAY10622 (3-[[[[[4-(aminocarbonyl) phenyl] amino] carbonyl]amino]methyl]-N-(1,2,3,4-tetrahydro-7-isoquinolinyl)-benzamide); Thiazovivin; GSK429286A; RKI-1447 (1-(3-Hydroxybenzyl)-3-(4-(pyridin-4-yl)thiazol-2-yl)urea); GSK180736A (GSK180736); Hydroxyfasudil (HA-1100); OXA 06; Y-39983; Netarsudil (AR-13324, see Lin et al., J Ocul Pharmacol Ther. 2018 Mar. 1; 34(1-2): 40-51, U.S. Pat. Nos. 8,450,344 and 8,394,826); GSK269962/GSK269962A; Fasudil (HA-1077, 1-(5-isoquinolinesulfonyl)-homopiperazine) and its derivatives such Ripasudil (K-115, 4-fluoro-5-[[(2S)-2-methyl-1,4-diazepan-1-yl]sulfonyl]isoquinoline; see WO1999/20620) and others that share the core structure of 5-(1,4-diazepan-1-ylsulfonyl)isoquinoline; KD025 (SLx-2119) and related compound and XD-4000 (see, e.g. Liao et al. 2007 J Cardiovasc Pharmacol 50:17-24; WO2010/104851 US 2012/0202793); SR 3677; AS 1892802; H-1152 ((S)-(+)-2-Methyl-1-[(4-methyl-5-isoquinolinyl)sulfonyl]homopiperazine, Ikenoya et al., J. Neurochem. 81:9, 2002; Sasaki et al., Pharmacol. Ther. 93:225, 2002); N-(4-Pyridyl)-N′-(2,4,6-trichlorophenyl)urea (Takami et al., Bioorg. Med. Chem. 12:2115, 2004); and 3-(4-Pyridyl)-1H-indole (Yarrow et al., Chem. Biol. 12:385, 2005); 3-[2-(aminomethyl)-5-[(pyridin-4-yl)carbamoyl]phenyl] benzoates including AMA0076 (compound 32, Boland et al., Bioorganic & Medicinal Chemistry Letters 23(23): 6442-6446 (2013)) TC-S 7001 and AT13148, and pharmaceutically acceptable salts thereof. Inhibitors with the scaffold 4-Phenyl-1H-pyrrolo [2,3-b] pyridine, including compound TS-f22, are described in Shen et al., Scientific Reports 5:16749 (2015). Other ROCK1/2 inhibitors include isoquinoline sulfonyl derivatives disclosed in WO 97/23222, Nature 389, 990-994 (1997) and WO 99/64011; heterocyclic amino derivatives disclosed in WO 01/56988; indazole derivatives disclosed in WO 02/100833; pyridylthiazole urea and other ROCK1/2 inhibitors as described in 20170049760; and quinazoline derivatives disclosed in WO 02/076976 and WO 02/076977; in WO02053143, p. 7, lines 1-5, EP1163910 A1, p. 3-6, WO02076976 A2, p. 4-9, preferably the compounds described on p. 10-13 and p. 14 lines 1-3, WO02/076977A2, the compounds I-VI of p. 4-5, WO03/082808, p. 3-p. 10 (until line 14), the indazole derivatives described in U.S. Pat. No. 7,563,906 B2, WO2005074643A2, p. 4-5 and the specific compounds of p. 10-11, WO2008015001, pages 4-6, EP1256574, claims 1-3, EP1270570, claims 1-4, and EP 1 550 660. These inhibitors are generally commercially available, e.g., from Santa Cruz Biotechnology, Selleck Chemicals, and Tocris, among others. For example, fasudil and Hydroxy fasudil are obtainable from Asahi Kasei Pharma Corp (Asano et al., J Pharmcol Exp Ther, 1987, 241(3): 1033-1040), Y-39983 is obtainable from Novartis/Senju (Fukiage et al., Biochem Biophys Res Commun, 2001, 288(2):296-300) and Y27632 is obtainable from Mitsubishi Pharma (Fu et al., FEBS Lett, 1998, 440(1-2):183-187). (S)-(+)-2-Methyl-1-[(4-methyl-5-isoquinolinyl) sulfonyl]homopiperazine], N-(4-Pyridyl)-N′-(2,4,6-trichlorophenyl) urea and 3-(4-Pyridyl)-1H-indole are also available at AXXORA (UK) Ltd and other suppliers. Protein or peptide inhibitor of ROCK1/2 are also known in the art, including inhibitors of ROCK1/2, e.g., a peptide consisting of 4-30 residues and exhibiting the sequence YSPS (SEQ ID NO:1), ERTYSPS (SEQ ID NO:2), or ERTYSPSTAVRS (SEQ ID NO:3)(see, e.g., US20170296617), or a kinase-defective mutant of ROCK1 or caspase 3 cleavage-resistant mutant of ROCK1 (e.g., as described in 2006/0142193). In some embodiments, the peptide further comprises one or more, e.g., all, D-amino acid residues.

### Raf Inhibitors

A number of RAF inhibitors are known in the art and used clinically, e.g., for the treatment of cancers. Exemplary RAF inhibitors include: SB590885; GDC0879; Vemurafenib; Dabrafenib; LGX818; AZ628; LY3009120; TAK632; MLN2480 (TAK580); BGB659; BGB283; CCT196969; CCT241161; CCT3833 (BAL3833); PLX7904; PLX8394; HM95573; CEP32496 (also known as RXDX-105); LXH254; RAF709; and BI 882370. See, e.g., Karoulia et al., Nat Rev Cancer. 2017 November; 17(11): 676-691; Jinghua et al., Cancer Management and Research Volume 10:2289-2301 (2018).

### GSK3β Inhibitors

Exemplary GSK3β inhibitors include CHIR-98023; CHIR-99021; CHIR-99030; Hymenialdisine; debromohymeialdisine; dibromocantherelline; Meridianine A; alsterpaullone; cazapaullone; Aloisine A; NSC 693868; (1H-Pyrazolo[3,4-b]quinoxalin-3-amine); Indirubin-3′-oxime; (Indirubin-3′-monoxime; 3-[1,3-Dihydro-3-(hydroxyimino)-2H-indol-2-ylidene]-1,3-dihydro-2H-indol-2-one); A 1070722; (1-(7-Methoxyquinolin-4-yl)-3-[6-(trifluoromethyl)pyridin-2-yl]urea); L803; L803-mts; TDZD8; NP00111; HMK-32; Manzamine A; Palinurin; Tricantin; IM-12; (3-(4-Fluorophenylethylamino)-1-methyl-4-(2-methyl-1H-indol-3-yl)-1H-pyrrole-2,5-dione); NP031112; NP00111; NP031115; VP 2.51; VP2.54; VP 3.16; and VP 3.35.

### Src Inhibitors

A number of Src inhibitors are known in the art, including WH4-023; Saracatinib (AZD0530); RK 24466; ENMD-2076; PRT062607 (P505-15) HCl; MG-47a; PP1; PP2; Src Inhibitor 1; and CCT196969. Other Src inhibitors include Dasatinib (e.g., Dasatinib hydrochloride or Dasatinib Monohydrate); Ponatinib (AP24534); Bosutinib (SKI-606); Pelitinib (EKB-569); Resveratrol; KX2-391 (Tirbanibulin); NVP-BHG712; PP121; MNS (3,4-Methylenedioxy-β-nitrostyrene); XL228; DGY-06-116; eCF506; 1-Naphthyl PP1(1-NA-PP1); AMG-47a; KX1-004; Myristic Acid; 7-Hydroxy-4-chromone; UM-164; Repotrectinib (TPX-0005); ON123300; SU6656; Doramapimod (BIRB 796); Dehydroabietic acid; Ginkgolic acid C17:1; AD80; and Quercetin. In some embodiments, a dual Lck/Src inhibitor is used.

### DNMT Inhibitors

In some embodiments, any of the treatments described herein are used in combination with an inhibitor of DNA methyltransferase (DNMT), e.g., DNMT1, DNMT3a/3b, etc. In some embodiments, the inhibitor of DNMT is RG 108, 5-azacytidine (also called “azacytidine”), decitabine (also called “5-Aza-2′-deoxycytidine”), Zebularine, procainamide, procaine, psammaplin A, sinefungin, temozolomide, OM173-alphaA, DNMT3A-binding protein, theaflavin 3,3′-digallate, 1-Hydrazinophthalazine, SGI-1027, hydralazine, NSC14778, Olsalazine, Nanaomycin, SID 49645275, Δ²-isoxazoline, epigallocatechin-3-gallate (EGCG), MG98, SGI-110, SGI-1027, SW155246, SW15524601, SW155246-2, or DZNep, an ASO targeting an DNMT, e.g. SEQ ID NO: 87, TCAAGTTGAGGCCAGAAGGA, or an siRNA targeting DNMT, optionally comprising SEQ ID NOs: 72-75. In some embodiments, an antisense oligonucleotide (ASO) comprises 12-50 nucleotides that binds to 12-50 consecutive nucleotides of a DNMT sequence. In some embodiments, an isolated antisense oligonucleotide (ASO) comprises at least one modification. In some embodiments, the at least one modification comprises one or more modified bonds or bases. In some embodiments, the modified bases comprise at least one ribonucleotide, at least one deoxyribonucleotide, or at least one bridged nucleotide, wherein the bridged nucleotide is a locked nucleic acid (LNA) nucleotide, a 2′-O-Ethyl (cEt) modified nucleotide, 2′-O-methoxy ethyl (MOE) nucleotide, or a 2′-O,4′-C-ethylene (ENA) modified nucleotide. In some embodiments, the modified bonds comprise phosphorothioate internucleotide linkages between at least two nucleotides, or between all nucleotides. In some embodiments, the ASO is a gapmer or mixer. In some embodiments, the ASO comprises unmodified deoxyribonucleosides in the center flanked by 5′ and 3′ terminal modified (e.g., bridged, locked) nucleosides. In some embodiments, comprises unmodified deoxyribonucleosides in the center flanked by 5′ and 3′ terminal modified (e.g., bridged, locked) nucleosides directs RNAse-H-mediated cleavage of a target DNMT transcript. In some embodiments, the locked nucleosides comprise a methylene bridge between the 2′-oxygen and the 4′-carbon. In some embodiments, there are 1, 2, 3, 4, 5, 6, 7, 8, 9, or 10 modified (e.g., bridged, locked) nucleosides at the 3′end. In some embodiments, there are 1, 2, 3, 4, 5, 6, 7, 8, 9, or 10 modified (e.g., bridged, locked) nucleosides at the 5′ end. In some embodiments, the modified nucleosides at the 3′ end and/or the 5′ end are 2′-O-methoxy ethyl (MOE) nucleotides. In some embodiments, the ASO comprises 1, 2, 3, 4, 5, 6, 7, 8, 9, or 10 2′-MOE nucleosides at the 3′ end and/or 1, 2, 3, 4, 5, 6, 7, 8, 9, or 10 2′-MOE nucleosides at the 5′ end.

### Methods of Generating Neural Precursors

The FMR1-reactivated iPSC can be subject to differentiation to neuronal precursor cells prior to administration. A number of neuronal differentiation protocols are known in the art; see, e.g., Chambers et al., Nat Biotechnol 27(3): 275-80 (dual SMAD inhibition); WO 2020/237104 (dopaminergic neuronal precursors); Salimi et al., Mol Biol Rep. 2014 March; 41(3):1713-21; Gunhnlar et al., Molecular Psychiatry 23:1336-1344 (2018); Trilck et al., Methods Mol Biol. 2016; 1353:233-59; Zhang et al., Stem Cell Res Ther. 2018 Mar. 15; 9(1):67; D'Aiuto et al., Organogenesis. 2014; 10(4):365-77; Marton and Ioannidis, Stem Cells Translational Medicine 2019; 8:366-374; Bell et al. Bio-protocol 9(5): e3188 (2019). DOI: 10.21769/BioProtoc.3188; Bianchi et al., Stem Cell Research 32:126-134 (2018). In some embodiments, the cells are excitatory neuronal cells (e.g., FMRP function has been reported to be important for CA1 pyramidal neurons in hippocampus; see Sawicka et al., eLife 2019; 8:e46919 DOI: 10.7554/eLife.46919).

### EXAMPLES

The invention is further described in the following examples, which do not limit the scope of the invention described in the claims.

### Methods

The following materials and methods were used in the Examples below.

### Cell Culture

WCMC37 hES cells (Colak et al., 2014) and 848-iPS1 (Sheridan et al., 2011) were maintained in mTeSR media with Matrigel-treated plates. Cells were passaged either with Accutase or TrypLE Select (Gibco) by following the manufacturer's manual. Feeder cells from UV-irradiated drug resistant mouse embryonic fibroblasts were used for the RSeT (Stem Cell Technology) or 5i/L/A (Theunissen et al., 2014) media condition, and TrypLE Select or Accutase was treated in 5-10 minutes until the hPSC cells start to be detached by a gentle tapping without disturbing feeder cells. 5i/L/A medium was generated by following the protocol from Theunissen et al 2014 [10]. Briefly, 240 ml DMEM/F12 (Gibco 10565-042), 240 ml Neurobasal media (Gibco 21103049), 1 mM glutamine (Gibco), 1% nonessential amino acids (Gibco), penicillin-streptomycin (Gibco), 50 mg/ml BSA (cell culture grade) were combined and used as a basal media. The following components were freshly added to 48.5 ml of basal media: 0.5 ml N2 supplement (Gibco 17502048), 1 ml B27 supplement (Gibco 17504044), 20 ng/ml recombinant human LIF (Peprotech), 0.1 mM b-mercaptoethanol (Sigma), 10 ng/ml Activin A (Peprotech), and the following small molecules from Naive Stem Cell 5i inhibitor Set (Axon 5011): PD0325901 (1 mM), IM-12 (1 mM), SB590885 (0.5 mM), WH-4-023 (1 mM), and Y-27632 (10 mM). For the composition optimization, the same basal media was used with different small molecule combinations. 40 μm sieve was used to collect the detached cells to minimize feeder contamination after TrypLE Select or Accutase is neutralized by DMEM media containing 10% FBS and 25 mM HEPES pH 7.0.

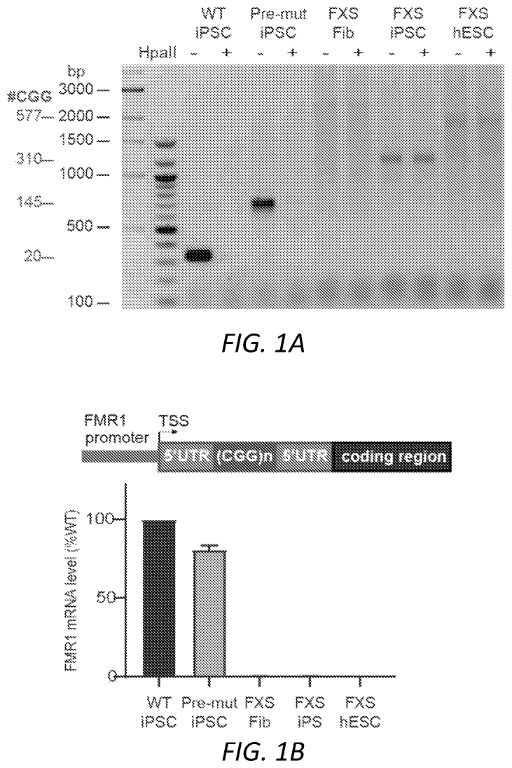

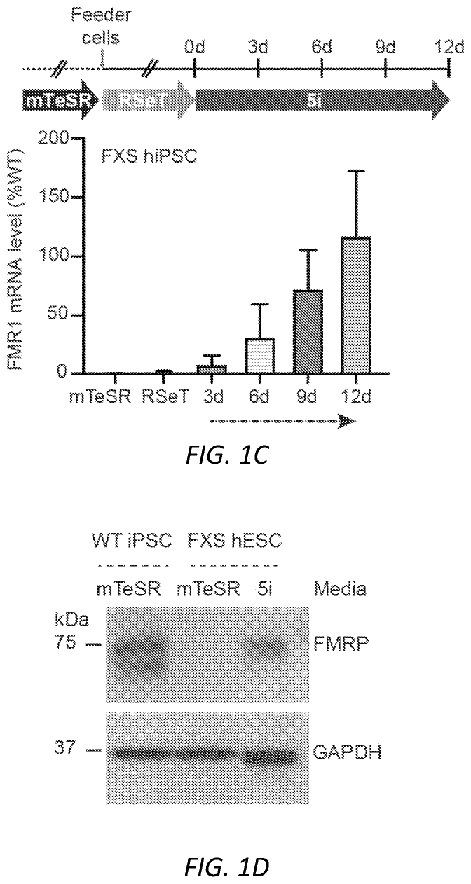

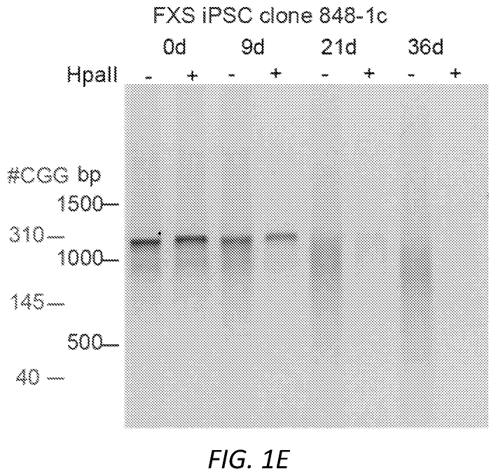

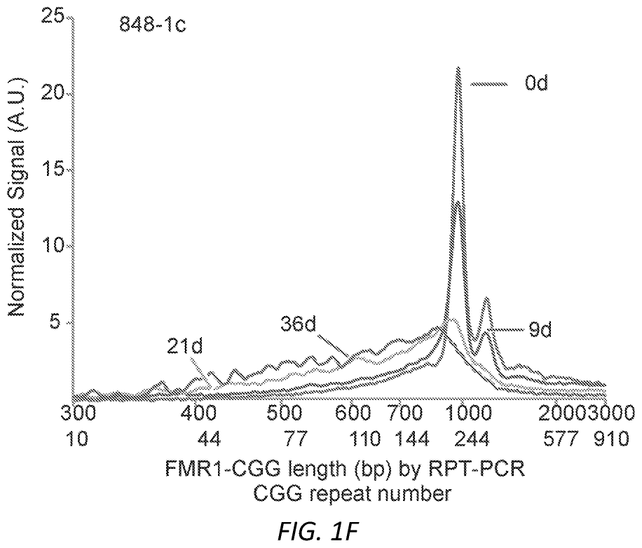

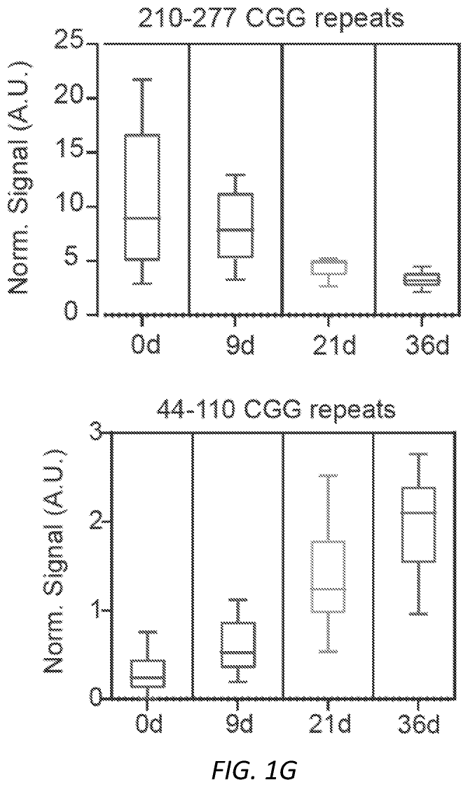

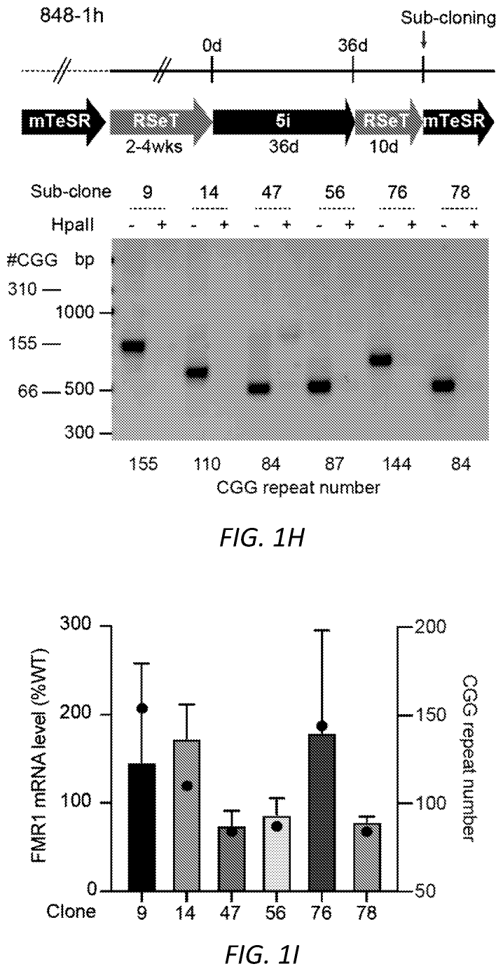

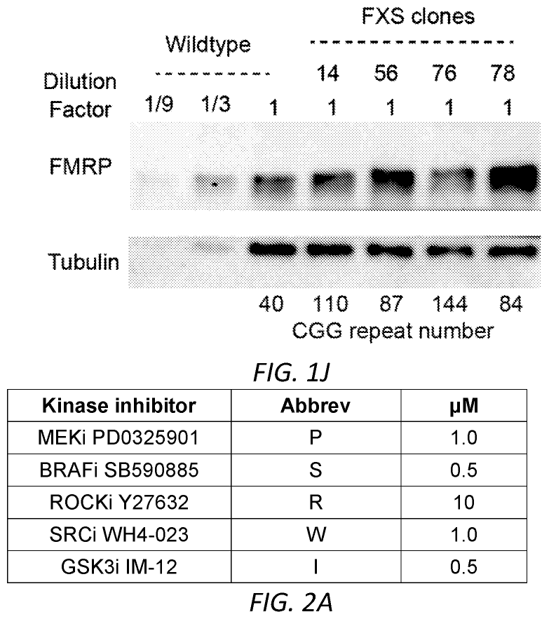

Experimental conditions for small molecule treatments were optimized to minimize the toxicity while sustaining the effects of the small molecules. As the direct conversion from regular human ES/iPS media (mTeSR) to 5i/L/A media caused a massive cell death, we first adopted the hES/iPS cells in RSeT media with feeder condition and gradually introduced 5i media by using 50% mixture of RSeT and 5i/L/A media. While FMR1 gene expression was not significantly increased during the long-term cultivation in RSeT media, treating the FXS hES/iPS cells with 50% 5i media triggered a significant increase in FMR1 gene expression in 6 days (FIG. 1C) without massive cell death.

Lentiviruses were produced by transfecting HEK293T cells with various dCas9-containing constructs or pgRNA constructs together with psPAX2 and pMD2.G-VSVG packaging vectors using Lipofectamine 3000 (Invitrogen). Fuw-dCas9-Tet1CD-P2A-BFP, Fuw-dCas9-dead Tet1CD-P2A-BFP, pgRNA-CGG and pgRNA-modified (for the backbone of the sgRNA construct) were gifts from Rudolf Jaenisch (Addgene plasmid #108245, #108246, #108248, #84477). pGH125 dCas9-Blast was a gift from Michael Bassik (Addgene plasmid #85417). Inducible dCas9 and dCas9-RNaseH1 plasmids were generated by modifying the pGH125 dCas9-Blast backbone (Addgene plasmid #85417). Briefly, EF1a promoter was swapped with TRE-tight Tet-responsive promoter of pTRE-Tight plasmid. The catalytic domain of human RNaseH1 (110-260 aa) was additionally subcloned into the 3′ end of dCas9 coding region as in-frame to create dCas9-RNaseH1 recombinant protein expressing cassette. Puromycin and Blasticidin are used for the selection markers of gRNA and dCas9-related constructs integration, respectively. Produced lentiviruses were further concentrated by Lenti-concentrator (Origene) and stored in Lenti-Stabilizer (Origene).

### RPT-PCR

Repeat-PCR (RPT-PCR), an assay for detecting the very long CGG repeats at the FMR1 locus, was performed as described in Hayward et al (Hayward et al., 2016). Due to the extremely high GC content and repetitive nature of the long CGG repeats, regular PCR protocols cannot be used for analyzing the length of the CGG repeats on full mutation FMR1 locus. Briefly, 600 ng of genomic DNA was treated with HindIII in 50 mM Tris-Cl pH 8.8, 1.5 mM MgCl2, 22 mM (NH4)2SO4, 0.2% Triton X-100 overnight at 37° C. EcoNI was added to remove the RPT-PCR product from mouse feeder cells. HpaII, a methylation-sensitive enzyme, was additionally included in the half of the HindIII digestion mix for monitoring the methylation status of the target regions. RPT-PCR master mix containing 50 mM Tris-HCl (pH 8.8), 1.5 mM MgCl2, 22 mM (NH4)2SO4, 0.2% Triton X-100, 3.3 M betaine (Sigma, St. Louis, MO), 2.67% dimethyl sulfoxide, 0.27 mM dNTPs, 27 U/ml High-Fidelity Phusion DNA polymerase (New England Biolabs) and 0.67 μM of primers (Not-FraxC and Not-FraxR4 for FMR1) was prepared on ice. For the RPT-PCR reactions, 5 μl of the HindIII-digested genomic DNA was added to 15 μl of the PCR master mix on ice and mixed well before PCR reaction. The PCR was performed by following cycles: 98° C. for 3 minutes, 30×(98° C. for 30 seconds, 64° C. for 30 seconds, 72° C. for 210 seconds), and 72° C. for 10 minutes. Since the RPT-PCR product includes 269 base pair flanking regions, the number of CGG repeat length can be calculated by the following formula:

number of CGG repeat=(fragment size−269)/3.

### Bioanalyzer Analysis

RPT-PCR products were purified by using PCR purification kit (Qiagen) and the purified samples were further analyzed by High sensitivity DNA kit with Bioanalyzer 2100 (Agilent) to quantify the proportion of long and short CGG fragments. Estimated fragment size was calculated by the migration time to size conversion based on the ladder migration. Raw signal data was normalized by the average intensity between 300 bp to 3 kb. Box plots are drawn by R package to show the signal intensity distribution on the long (900-1100 bp: 210-277 CGG repeats) and short (400-600 bp: 44-110 CGG repeats) CGG repeat range.

### DRIP (DNA-RNA Hybrid Immunoprecipitation)

R-loop formation (DNA-RNA hybrid) was monitored by DRIP. DRIP assay was performed by following the protocol from Loomis et al (Loomis et al., 2014) with slight modifications. Briefly, cell pellets were resuspended in 10 mM Tris-HCl, 10 mM EDTA, 100 mM NaCl pH 8, lysed with 0.5% SDS, and digested with 400 units of Proteinase K overnight at 37° C. Cell lysates were then extracted once with phenol pH 8 and twice with chloroform. DNA was precipitated with 1 volume of isopropanol and 300 mM sodium acetate, and the pellet was washed twice with 70% ethanol, and was resuspended in 10 mM Tris-HCl pH 8. Harvested nucleic acids (˜10 ug) were digested with EcoRI, HindIII, BsrGI, and XbaI (20 units each) overnight at 37° C. in NEBuffer 2. RNaseH1, which degrades RNA-DNA hybrid, was treated to test the specificity of the DRIP-qPCR signal and served as negative control. After that, samples were purified by phenol and chloroform extraction followed by precipitation in isopropanol. The pellet was washed twice with 70% ethanol. Air-dried pellets were resuspended in 10 mM Tris-HCl pH 7.5, 1 mM EDTA.

Digested nucleic acids (2-5 ug) were diluted in 450 μL of TE, and 10 μL was reserved as input for qPCR. 10×IP buffer was added for a final buffer concentration of 10 mM sodium phosphate, 140 mM sodium chloride, 0.05% Triton X-100, and 20 μL of S9.6 antibody was added and incubated at 4° C. for 2 hours. After the antibody incubation, Protein A/G magnetic beads were washed twice with 800 μL of 1×IP buffer for 5 minutes at room temperature and 40 μl of the beads were added to each sample and incubated for 2 hours at 4° C. Beads were washed three times with 700 μL 1×IP buffer for 10 minutes at room temperature. After the wash, the magnetic beads were resuspended in 250 μL of 1×IP buffer and incubated with 60 units of Proteinase K for 30 minutes at 50° C. Digested DRIP samples were purified by phenol/chloroform extraction and isopropanol precipitation. Pellets were resuspended in 80 μL of 10 mM Tris-HCl pH 8.0.

### Methylated DNA Immunoprecipitation (MeDIP) and Pyrosequencing

5 mC and 5 hmC levels on FMR1 locus were measured by qPCR assay with the pull down material of methylated DNA by antibody recognizing 5 mC (Active Motif monoclonal Ab clone 33D3) and 5 hmC (Active Motif polyclonal Ab #39769). Briefly, genomic DNA was purified from cultured cells by overnight Proteinase K treatment, followed by phenol-chloroform extraction and ethanol precipitation. DNA was fragmented using HindIII, EcoRI, BsrGI, and XbaI at 37 C overnight. We used 2-4 μg of fragmented DNA for a standard MeDIP assay. After denaturing by incubating at 95° C. for 10 min, the sample was immunoprecipitated for 2 h at 4° C. with 10 μl of monoclonal antibody against 5-methylcytidine ( ) in a final volume of 500 μl IP buffer (10 mM sodium phosphate (pH 7.0), 140 mM NaCl, 0.05% Triton X-100). We incubated the mixture with 30 μl of Dynabeads for 2 h at 4° C. and washed it three times with 700 μl of IP buffer. Beads were treated with proteinase K for 3 h at 56° C. and pull-downed methylated DNA was purified by phenol-chloroform extraction and ethanol precipitation. Pyrosequencing was performed by EpigenDx (ADS1451-FS2).

### Chromatin Immunoprecipitation (ChIP)

Harvested cells are fixed in 1% formaldehyde for 5 minutes at room temperature, and glycine was added to a final conc. of 0.125M and incubated for 5 minutes at room temperature to stop crosslinking. After washing twice with cold PBS, cross-linked cells were resuspended in Buffer 1 (50 mM HEPES-KOH PH 7.5, 150 mM NaCl, 1 mM EDTA pH 8.0, 0.5% NP40, 0.25% Triton X-100 with freshly added protease inhibitor cocktail (PIC)) and rotated at 4 C for 10 min. After spin-down at 1700 rcf for 5 min in 4 C, cells were resuspended in Buffer 2 (10 mM Tris-Cl pH 8.0, 200 mM NaCl, 5 mM EDTA pH 8.0, 2.5 mM EGTA pH 8.0 with freshly added PIC) and rotated on a wheel for 10 min at 4C. Finally, cells were incubated in Buffer 3 (10 mM Tris-Cl pH 8.0, 5 mM EDTA pH 8.0, 2.5 mM EGTA pH 8.0 with freshly added PIC) with RNase A and rotated on a wheel for 30 min at 37 C. After adding N-lauroyl sarcosine to final conc. 0.5%, samples were sonicated by using Covaris system with 15×19 mm tube, 5% duty cycle, intensity 140, and 200 burst per cycle for 10-15 min. Sonicated samples were centrifuged by 14000 rpm for 10 min in 4 C and supernatant was used for immunoprecipitation and input samples. For immunoprecipitation, equal volume of 2×IP buffer (2% Triton X-100, 300 mM NaCl, 30 mM Tris-HCl pH 8.0, 1×PIC) was added to the sonicated samples and 2-5 μg of antibody was added per IP and incubated o/n at 4 C on a rotating wheel. 40 μl of Protein G Dynabeads was washed twice with Buffer1 and added to each IP sample and incubated for 2 hrs at 4 C on a rotating wheel. Beads were washed 3 times with ½×RIPA-1 buffer (50 mM HEPES-KOH pH 7.5, 500 mM NaCl, 10 mM EDTA pH 8.0, 1% NP40, 0.5% Sodium Deoxycholate) and 3 times with RIPA-3 buffer (50 mM HEPES-KOH PH 7.5, 50 mM NaCl, 10 mM EDTA pH 8.0, 0.2% NP40) at 4 C for 5 min on a rotating wheel. Washed beads were resuspended to 200 μl TES buffer (50 mM Tris-Cl pH 8.0, 10 mM EDTA pH 8.0, and 1% SDS) and incubated at 65 C for 15 min. After spin down, 40 μg of Proteinase K was added to the supernatant and incubated in 65 C for 4 hrs to O/N for reversing the crosslinking. After phenol/chloroform extraction and ethanol precipitation with glycogen, resulted pellets were resuspended in dH2O and analyzed by qPCR assay with purified input samples.

### RT-qPCR

RNAzol was treated to the collected cell pellets, and total RNA was extracted by following the manufacturer's manual. Reverse transcription reaction was performed by using QuantiTect (Qiagen). Reverse transcribed cDNA was used for quantitative PCR (qPCR) with 2×SyBr Green qPCR mix. GAPDH was used for normalization between samples. The list of primers and their sequences can be found in Supplemental Table S1.

### Western Blot Analysis

Total whole cell extract was prepared by first lysing cells in Lysis buffer containing 50 mM Tris-Cl (pH 8.0), 150 mM NaCl, 1% Triton X-100, 0.5% sodium deoxycholate, and 0.1% SDS. Samples were quantified by Bradford assay for even loading. Serial dilutions of cell lysates were loaded on a NuPAGE 4-12% or BioRad 4-20% gradient SDS polyacrylamide gel, separated by electrophoresis, transferred to a PVDF membrane. After 1 hr blocking with 5% skim milk in PBST (1×PBS, and 0.1% Tween-20), the membrane was incubated with primary antibodies for overnight at 4° C. and then secondary antibodies (1:20,000) at room temperature for 30 min with 1% skim milk in PBST followed by two washing steps. Primary antibodies were used with the following dilutions-1:2000 for anti-FMRP 1c3 (Millipore-Sigma MAB2160), 1:5000 for anti-tubulin (Sigma-Aldrich T5201) and anti-GAPDH.

### Knock-Down Assays

To degrade the nascent FMR1 transcript by RNase H1 activity, FMR1 ASO gapmer that has 10 bp 2′deoxy part (DNA) in the middle of 5 nt 2-methoxyethyl (2′ MOE) each side was used. FMR1-all-2MOE was used as a negative control which has the same sequence but the whole 20 nt sequence is 2′ MOE—no 2′deoxy part in the middle that triggers RNase H activity for gapmers. Control ASO is a scrambled gapmer ASO. For DNMT1, MSH2, and CSB (also known as ERCC6) knock-down, ON-TARGETplus human siRNA SmartPool (Dharmacon) sets were used. Lipofectamine 3000 was used for introducing ASOs (20 nM final conc.) and siRNAs (45-90 nM final conc.) into the cells the day after splitting every 3 days.

### Transcriptome Analysis by RNA-seq

The whole transcriptome analysis was performed by making RNA-seq libraries from Poly(A) selected RNA samples using NEBNext Poly(A) mRNA Magnetic Isolation Module (#E7490) and NEBNext Ultra™ II Directional RNA Library Prep Kit for Illumina (#E7760S) by following the protocols from the manufacturer. DEseq2 (Love et al., 2014), featureCounts (Liao et al., 2014), and enhanced Volcano were used for analyzing the differentially expressed genes.

### Statistical Analysis

We conducted t-test for most of the statistical analysis if not specified. Statistical significance is indicated by the following notations: ns for P>0.05, * for P≤0.05, ** for P≤0.01, *** for P≤0.001, and **** for P≤0.0001.

### Example 1: FMR1 Reactivation by Cellular Reprogramming

To study FXS repeat contraction, we used two established models derived from FXS patients: (i) a human induced pluripotent stem cell (hiPSC) line from a FXS full mutation male (848-iPS1) (Sheridan et al., 2011), and (ii) a human embryonic stem cell (hESC) line outgrown from an IVF-cultured blastocyst with a FXS full mutation (WCMC37)(Colak et al., 2014). Using established repeat-PCR (RPT-PCR) assays for long CGG repeats (Hayward et al., 2016), we confirmed the lengths of CGG repeats in various FXS and WT hiPSCs (FIG. 1A). Previous studies have shown that FMR1 is generally silent in FXS hiPSCs (Sheridan et al., 2011; Mor-Shaked and Eiges, 2016). By contrast, FMR1 was shown to be expressed in early passage FXS hESCs (Colak et al., 2014), though the gene was also shown to be hypermethylated and silenced in other hESC lines (Mor-Shaked and Eiges, 2016). We conducted experiments to test whether prolonged in vitro culturing could impact the epigenetic state of FMR1 chromatin. The results showed that the hESCs and hiPSCs—which were obtained as later-passage lines—showed FMR1 silencing (FIG. 1B). Resistance to digestion by the methylation-sensitive HpaII restriction enzyme indicated that the CGG's were methylated (FIG. 1A). Because early passage FXS hESCs can express FMR1 (Colak et al., 2014), we conducted experiments to test whether that loss of the so-called ‘naïve state’ (that often happens to hESCs during prolonged culture; Gafni et al., 2013; Theunissen et al., 2014), could have changed their epigenetic state, rendering them more similar to developmentally downstream epiblast stem cells. One previous work showed that culturing FXS hiPSCs in Naïve Human Stem cell Media (NHSM) promoted the naïve state and resulted in FMR1 reactivation (Gafni et al., 2013), but another study showed that culturing in NHSM did not cause FMR1 re-expression from FXS hiPSCs (de Esch et al., 2014).

To investigate in our system, we cultured FXS hESC and hiPSC in RSeT media, a modified commercial version of NHSM. We did not observe any significant FMR1 reactivation after growing the cells in RSeT (FIG. 1C). Thus, culturing in NHSM-based naïve state media per se does not invariably lead to FMR1 reactivation. We tested another naïve state formulation dubbed “5i” (Theunissen et al., 2014). To avoid cell shock caused by a sudden change in media, we transitioned cells by firstly co-culturing with feeder cells in RSeT and then gradually titrating in 5i media at 50:50 ratio to RSeT (FIG. 1C). Intriguingly, we observed a progressive increase in FMR1 mRNA expression, first detectable at 3-6 days of culture and achieving full reactivation by day 12. Introducing RSeT media as an intermediate step enabled cells to adapt more easily to 5i conditions and yielded consistently faster FMR1 reactivation.

The 5i media includes a combination of five compounds, including inhibitors of MEK, GSK3β, BRAF, ROCK, and SRC, and supports the expansion of viable OCT4−DPE−GFP+ human pluripotent cells after exogenous transcription factor expression has been removed (Theunissen, T. W., Powell, B. E., Wang, H., Mitalipova, M., Faddah, D. A., Reddy, J., Fan, Z. P., Maetzel, D., Ganz, K., Shi, L., et al. (2014). Systematic Identification of Culture Conditions for Induction and Maintenance of Naive Human Pluripotency. Cell Stem Cell 15, 524-526.).

Individuals with “pre-mutation” (PM) alleles have 50-200 CGG repeats but have either normal or considerably elevated mRNA levels (Tassone et al., 2007; Sheridan et al., 2011; Ludwig et al., 2014). Despite this, PM alleles do not have elevated FMRP protein (Primerano et al., 2002; Sheridan et al., 2011; Ludwig et al., 2014). To determine whether the protein was also restored in the 5i-treated cells, we performed Western blot analysis. The results showed FMRP production was restored to normal levels (FIG. 1D). Together, these data identify new growth conditions with specific media formulation in which FMR1 can be reactivated robustly.

### Example 2: FMR1 Reactivation is Accompanied by CGG Repeat Contraction

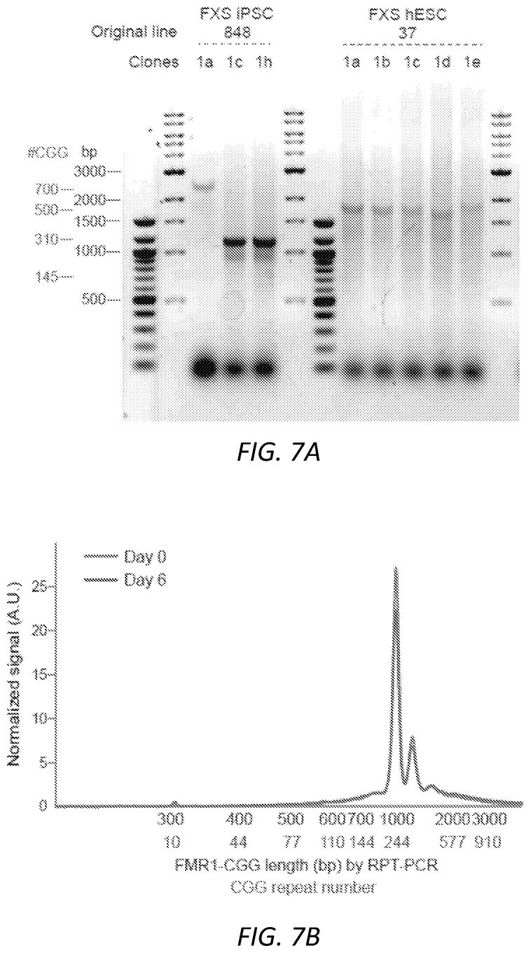

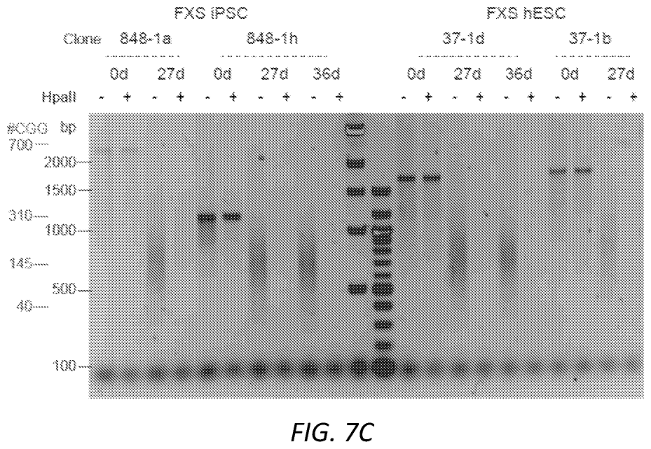

In wildtype cells, FMR1 expression is associated with low CGG repeat numbers and CpG hypomethylation. We asked if the CGG repeat numbers have changed in the reactivated FXS cells exposed to 5i. We derived single-cell clones from the FXS hESC and hiPSC lines to ensure that experiments were performed on cells initially carrying a homogeneous full mutation allele and silent FMR1 (FIG. 7A). We then examined hiPSC clone 848-1c, which carried approximately 310 repeats. RPT-PCR analysis showed that, intriguingly, FMR1 reactivation was consistently accompanied by a progressive shortening of the full-mutation CGG repeats (FIG. 1E). The shortening was partially evident by day 9 and continued at least through day 36. To quantitate CGG repeat lengths, we conducted a time-series RPT-PCR coupled to Bioanalyzer analysis (FIG. 1F) and sampled the population of long (210-277×) versus short (44-110×) CGGs across 36 days (FIG. 1G). Notably, although FMR1 was significantly reactivated by day 6 (FIG. 1C), the FM CGG repeat appeared mostly intact at this time (FIG. 7B). Contraction was gradual and appeared to follow, rather than precede, FMR1 reactivation.

RPT-PCR analysis also revealed that reactivation was accompanied by DNA demethylation. We found that pre-treating genomic DNA with HpaII showed a progressive sensitivity to HpaII during FMR1 reactivation after day 9 when FMR1 was strongly activated (FIGS. 1C and 1E). This observation indicated that DNA demethylation occurs at least contemporaneously with FMR1 reactivation. This finding is also consistent with a previous study in which DNA demethylation induced by targeting TET1 to FMR1 resulted in gene reactivation in FXS cells (Liu et al., 2018). 5i treatment resulted in progressive shortening and a range of shorter repeats. At 36 days of treatment, the majority of the CGG repeat numbers ranged from 200 copies to less than 100 copies (FIGS. 1E-1G), thereby encompassing a range of repeats in the pre-mutation range. Notably, because repeat numbers were estimated by intensity of a DNA-intercalated dye (e.g. Ethidium Bromide), this method intrinsically underestimates representation of shorter repeats, as shorter repeats intercalate less dye. Thus, shorter repeats in the low PM range (50-100×) and normal range (<50×) could both be present in significant numbers.

To rule out clonal artifacts, we repeated the experiments using additional hiPSC clones as well as new hESC clones (FIGS. 7A,7C). hESC clones 37-1b and 37-1d have 450-500 repeats, whereas hiPSC clones 848-1a and 848-1h have 650-700 and ˜310 repeats, respectively. After 5i treatment for 27-36 days, each clone demonstrated strong demethylation and repeat contraction (FIG. 7C). In the HpaII-minus samples, a smear of CGG repeat lengths could be observed in each case, indicating a shortening of CGG repeats to variable lengths within the otherwise clonal population. With HpaII treatment, the DNA became undetectable, consistent with HpaII digestion of the unmethylated DNA. These data indicate that CGG demethylation and contraction are generally observed in all tested clones that initially carried FM FXS repeats. To also exclude the possibility that RPT-PCR smearing observed during 5i treatment resulted from a PCR artifact, we sub-cloned 848-1h after a 36-day 5i treatment and examined lengths of the resulting CGG repeat in each sub-clone (FIG. 1H). Indeed, a range of CGG repeat numbers was observed, with some as low as ˜80 repeats (subclones 47, 56, 78).

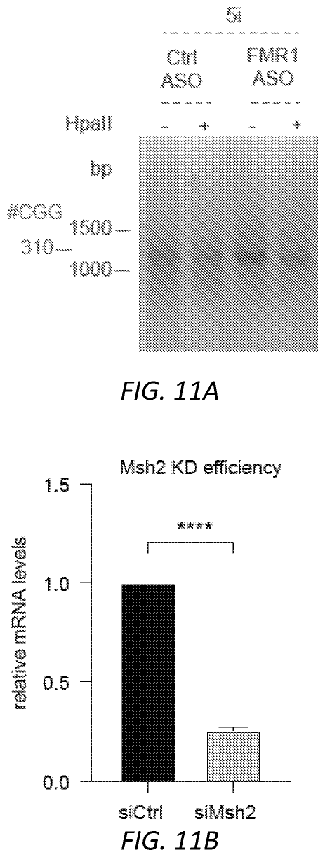

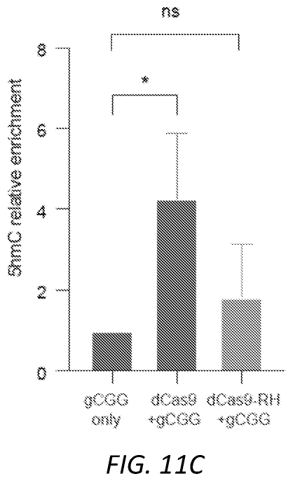

When FM cells were induced by 5i to contract to 50-200 repeat lengths, the sub-clones showed high-level mRNA expression consistent with levels observed in PM cell lines (FIG. 11), in stark contrast to the parental cell 848 hiPSC line (FIG. 1B). Furthermore, each subclone showed FMRP protein restoration to the level comparable to wild-type iPS cells, as determined by Western blotting with anti-FMRP antibodies (FIG. 1J). We noted a correlation between repeat copy number and mRNA expression, such that lower-copy repeats showed nearly normal mRNA levels (subclones 47, 56, 78), whereas higher-copy repeats tend to show higher mRNA levels (subclones 9, 14, 76). We conclude that 5i-induced CGG contraction results not only in mRNA expression but also expression of the protein product.

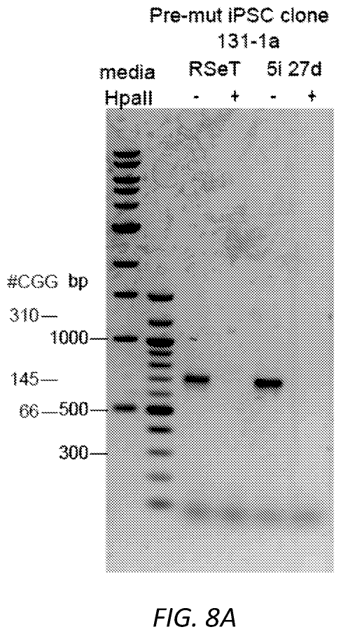

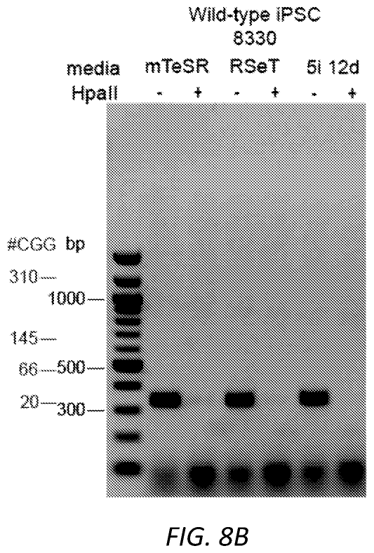

Given these results, we were curious about whether PM cells would also be responsive to 5i treatment, or whether the treatment is only effective in FM cells. We derived clones from a PM iPSC line, 131, and examined clone 131-1a which carried ˜150 CGG repeats. No obvious repeat contraction was observed after 27 days of 5i treatment (FIG. 8A). Similarly, no repeat contraction was observed from wild-type iPSCs after 12 days of 5i treatment (FIG. 8B). Intriguingly, there thus appears to be a copy number threshold of ˜150 CGG below which 5i cannot initiate contraction.

### Example 3: Repeat Contraction Attributed to MEK and BRAF Inhibition

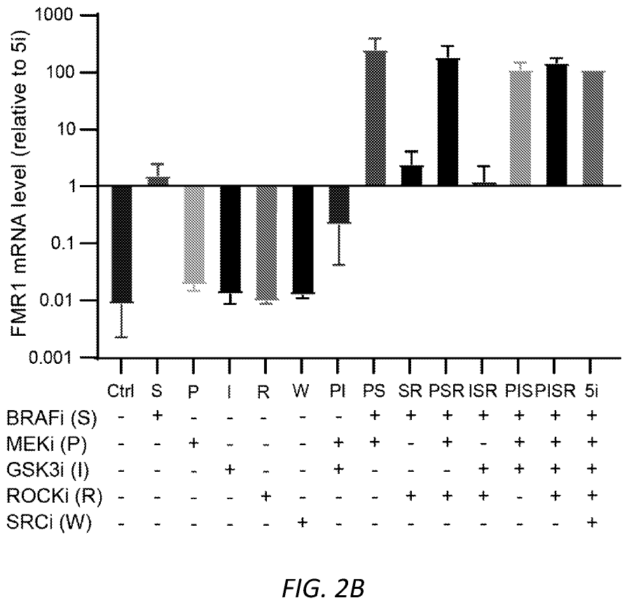

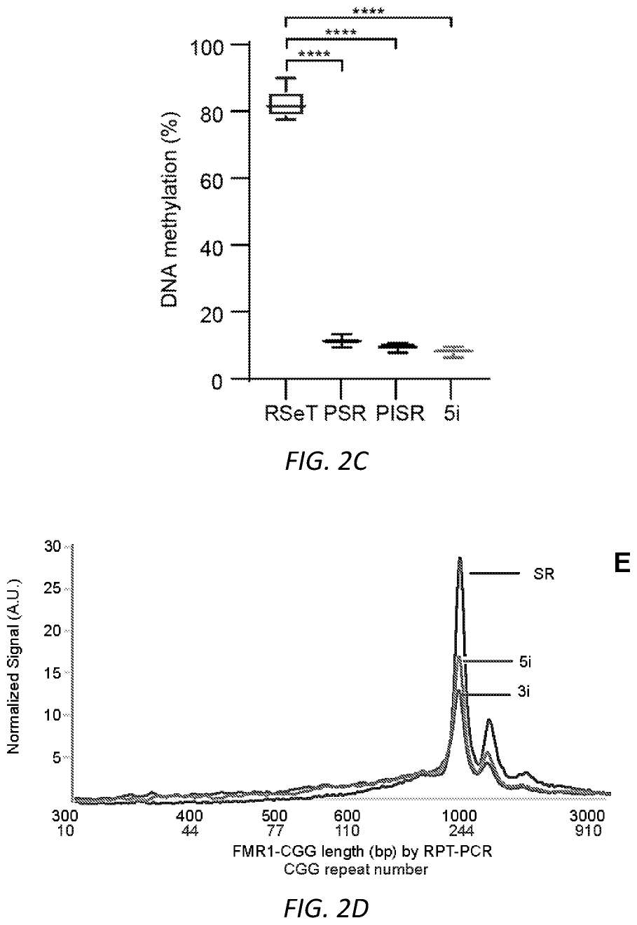

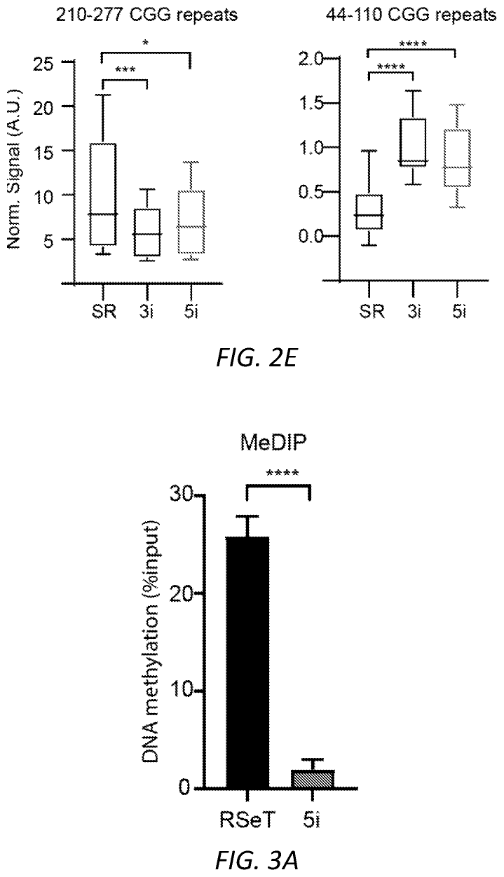

As discussed above and depicted in FIG. 2A, 5i media contains a mixture of 5 small molecule inhibitors (“i”) of various kinases: PD0325901 (“P”, MEKi), IM-12 (“G”, GSK3i), SB590885 (“S”, BRAFi), Y27632 (“R”, ROCKi), and WH4-023 (“W”, SRCi)—a combination previously shown to promote the pluripotent naïve state (Theunissen et al., 2014).

TABLE 4
Small Molecules.

| | Exemplary | Initials used | Final conc. |
|---|---|---|---|
| Small molecule target | Molecule | in FIGS. | (μM) |
| MEK Inhibitor | PD0325901 | P | 1 |
| ROCK1 inhibitor | ROCKi Y27632 | R | 10 |
| Raf inhibitor | SB590885 | S | 0.5 |
| GSK-3β inhibitor | IM-12 | I | 0.5 |
| Src (or dual Lck/Src) | WH4-023 | W | 1 |
| Inhibitor | | | |

We tested which of the 5 inhibitors is responsible for reactivating FMR1. We treated 848-1c hiPSCs with a single inhibitor or various combinations of the 5 small molecules without changing other components of the base media. Critically, no single inhibitor alone was sufficient to trigger reactivation (FIG. 2B). On the other hand, the combination of two inhibitors, MEKi and BRAFi (P+S), was intriguingly effective at triggering full FMR1 expression (FIG. 2B). Although MEK and BRAF kinases belong to the same MAPK pathway, targeting either kinase alone did not trigger reactivation (FIG. 2B), suggesting that MEK and BRAF have downstream targets that do not overlap and that would be necessary to achieve FMR1 reactivation. The P+S combination, however, caused slower cell growth. Addition of ROCKi (P+S+R) restored cell proliferation without negative effects on FMR1 reactivation (FIG. 2B). Thus, while the ROCKi was not essential for FMR1 reactivation, its addition promoted hiPSC viability during cell passaging, consistent with its original use (Watanabe et al., 2007). Other combinations, including S+R, S+R+I, and P+I did not substantially increase FMR1 expression. In cells treated with P+S+R (henceforth “3i”), pyrosequencing revealed the same degree of CpG demethylation in the FMR1 promoter region as compared to the full 5i treatment (FIGS. 2C,9A). Furthermore, RPT-PCR demonstrated CGG repeat contraction at least as good as that observed with full 5i treatment (FIGS. 2D,2E). We conclude that the combination of MEKi+BRAFi is sufficient to trigger DNA demethylation, CGG contraction, and FMR1 reactivation.

### Example 4: A Role for DNA Demethylation

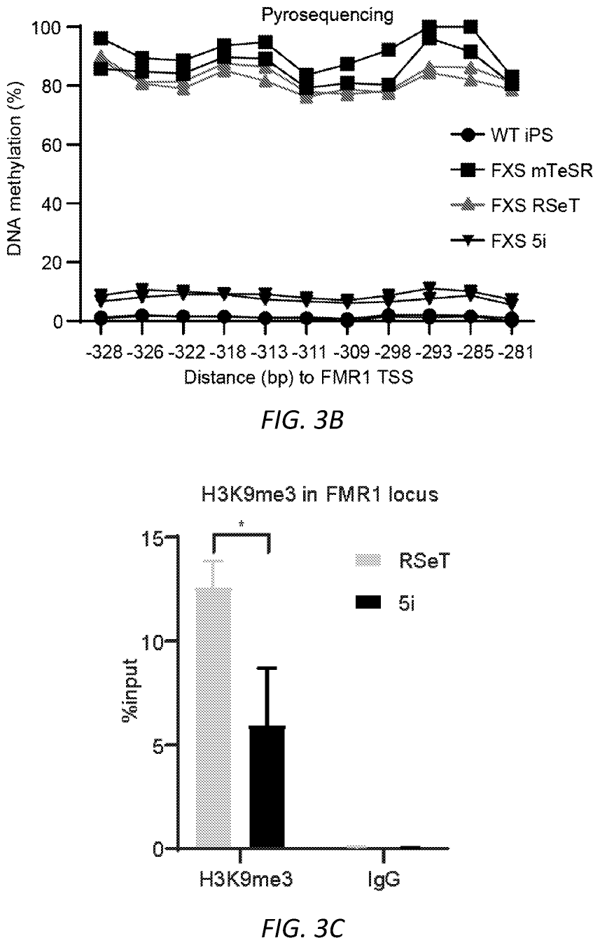

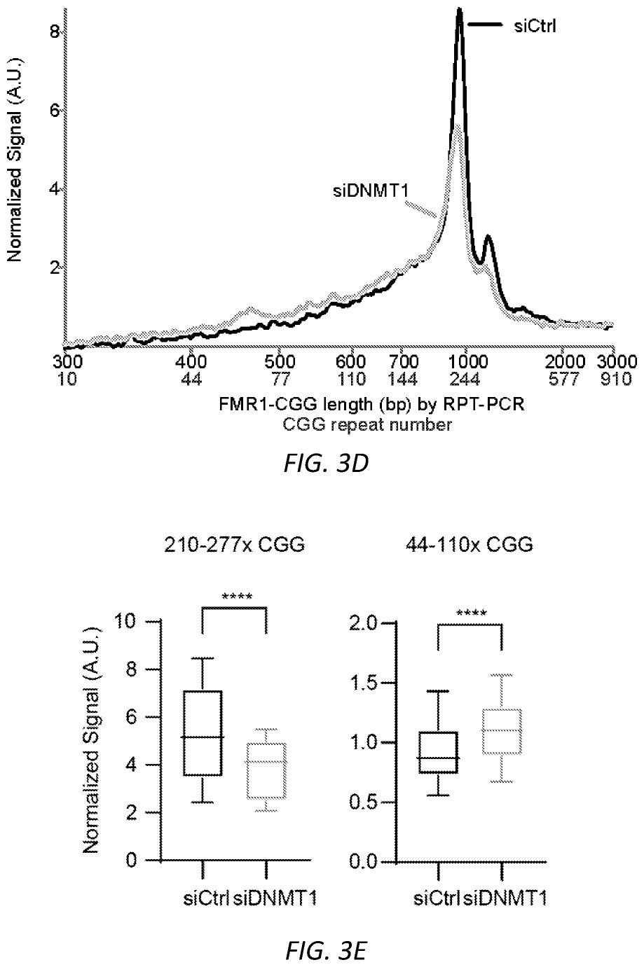

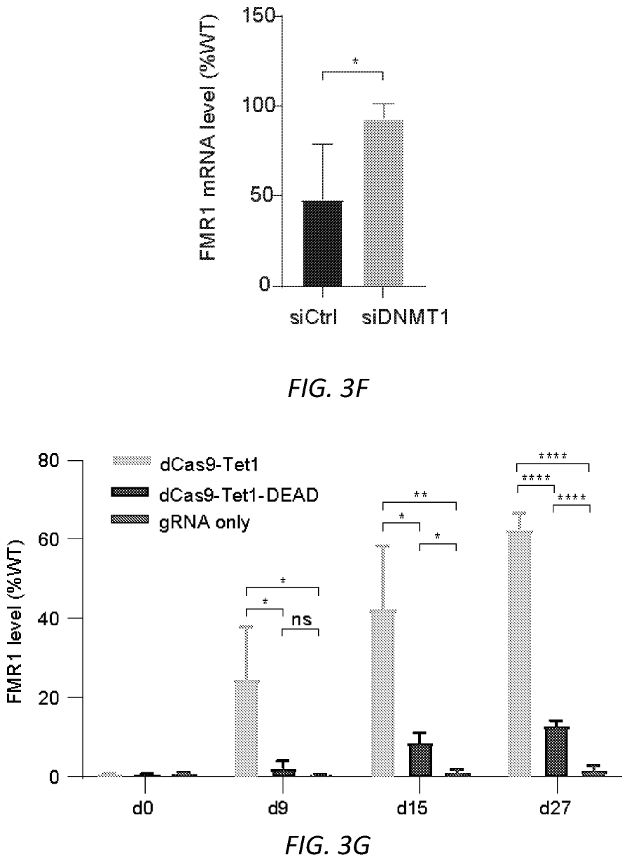

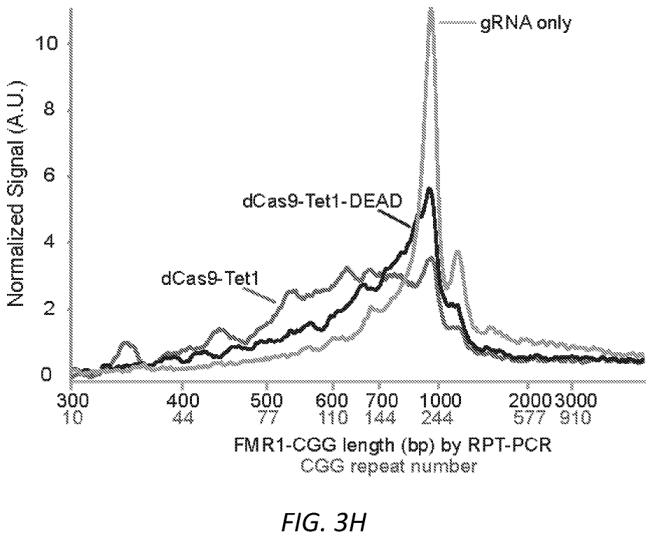

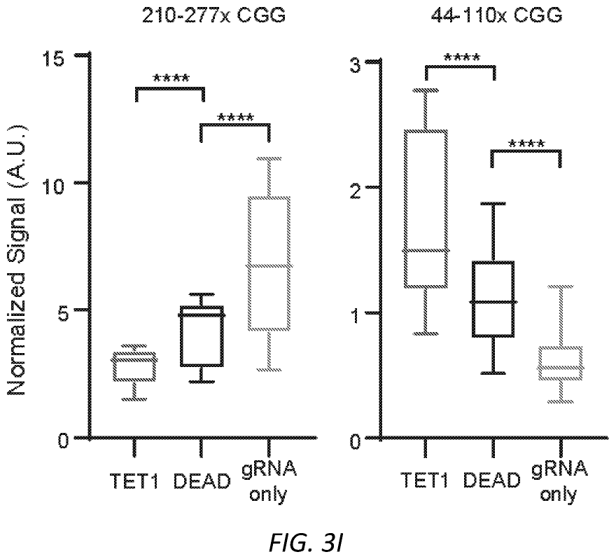

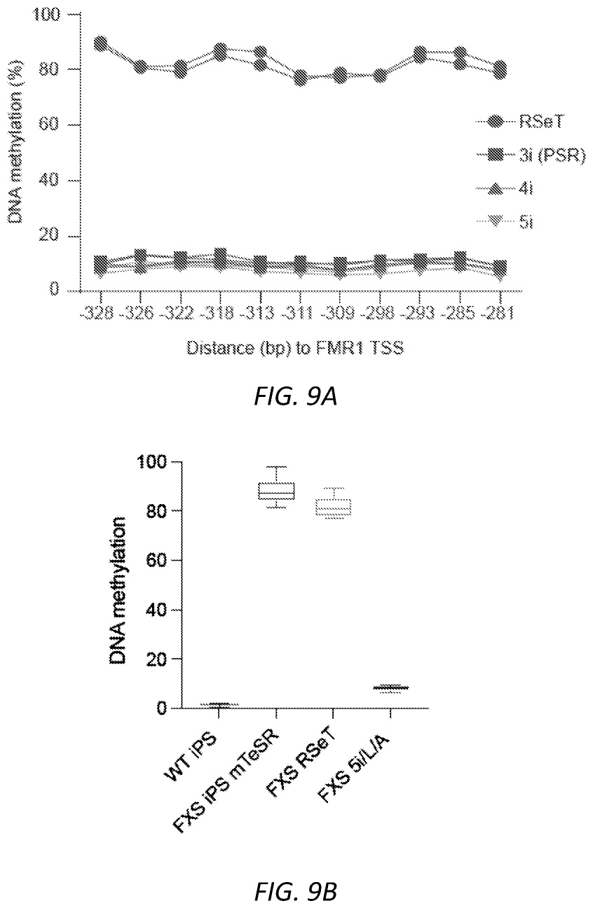

The loss of DNA methylation concurrent with CGG contraction raised the possibility that DNA demethylation may be involved mechanistically. We confirmed DNA demethylation using several orthogonal methods. MeDIP is an immunoprecipitation method that captures methylated DNA using 5-methyl-C (5 mC) antibodies. MeDIP showed a strong loss of DNA methylation in the FMR1 promoter following 5i treatment for 9 days, as compared to cells grown in RSeT (FIG. 3A). To obtain single-nucleotide resolution, we also performed pyrosequencing, a sequencing-by-synthesis method that accurately quantitates DNA methylation levels (FIGS. 3B, 9B). Pyrosequencing detected a strong loss of methylation across all CpGs examined within the FMR1 promoter region after 5i treatment for 12 days, with the unmethylated state resembling the native state of WT iPSCs (FIG. 3B). The CpG demethylation was specific to the 5i treatment and did not occur in FXS cells grown in mTeSR or RSeT. By contrast, there was not a dramatic loss of H3K9me3, a repressive chromatin mark strongly associated with the FM allele (Coffee et al., 2002). ChIP-qPCR analysis revealed a robust H3K9me3 enrichment in FM cells grown in RSeT media (FIG. 3C). Exposure to 5i moderately, but significantly, reduced H3K9me3 to ˜50%, despite strong FMRI reactivation after 6 days (FIG. 3C). This observation suggests that residual H3K9me3 mark is compatible with high level FMR1 expression on day 6. We conclude that FMRI reactivation is associated with loss of promoter CpG methylation but does not require removal of all H3K9me3 marks.

We then tested if depleting DNMT1, the enzyme responsible for maintenance of CpG methylation, might trigger contraction and reactivation. Because treating with the DNA methylation inhibitor, decitabine, is toxic to hiPSCs, we used an RNAi to knock down DNMT1, treating 848-1c hiPSCs with either a DNMT1-specific siRNA or a control scramble siRNA (siCtrl) for 6 days in RSeT media. The DNMT1-specific siRNA from Dharmacon (catalog no.: L-004605-00-0005) which comprised the following four siRNAs:

(SEQ ID NO: 72)
GCACCUCAUUUGCCGAAUA
(SEQ ID NO: 73)
AUAAAUGAAUGGUGGAUCA
(SEQ ID NO: 74)
CCUGAGCCCUACCGAAUUG
(SEQ ID NO: 75)
GGACGACCCUGACCUCAAA

The control non-targeting siRNA (also from Dharmacon catalog no.: D-001810-10-05) comprised the four following siRNAs:

(SEQ ID NO: 76)
UGGUUUACAUGUCGACUAA
(SEQ ID NO: 77)
UGGUUUACAUGUUGUGUGA
(SEQ ID NO: 78)
UGGUUUACAUGUUUUCUGA
(SEQ ID NO: 79)
UGGUUUACAUGUUUUCCUA

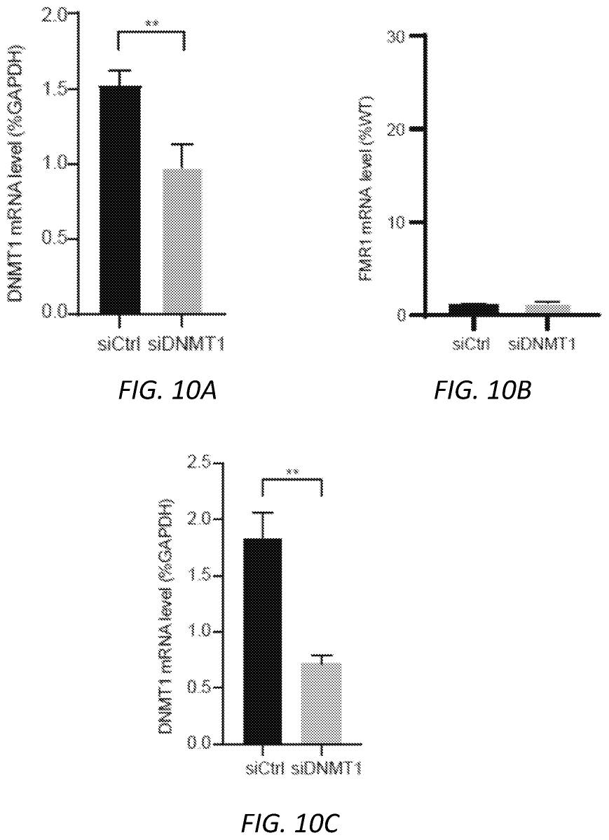

Despite a partial knockdown of DNMT1, we observed no FMR1 reactivation (FIGS. 10A,10B). When RNAi was repeated in 5i media, the DNMT1 knockdown led to a modest but significant CGG contraction, as shown by RPT-PCR and bioanalyzer quantitation (FIGS. 3D,3E,10C). In the range of 210-277 repeats, there was a significant drop in representation, whereas there was a significant increase in the short repeat range of 44-110×CGGs (FIG. 3E). Concurrently, there was a ˜2-fold increase in FMR1 mRNA with 5i/siDNMT1 treatment relative to 5i/siCtrl treatment on day 6 (FIG. 3F), indicating faster and stronger FMR1 reactivation when DNMT1 was partially depleted in 5i-treated cells. Together, these data indicate that DNMT1 inhibition can significantly potentiate contraction and reactivation in the presence of 5i, supporting the involvement of CpG demethylation in the reactivation mechanism.

### Example 5: Site-Specific R-Loops Trigger CGG Repeat Contraction

To investigate additional molecular requirements for CGG contraction, we considered a published observation in which targeting TET1 demethylase to FMR1 yielded FMR1 reactivation (Liu et al., 2018). We designed experiments to target a TET1-tethered catalytically dead Cas9 (dCas9-Tet1) to FMR1 using CGG-specific gRNAs. In 848-1c cells grown in mTeSR, we observed a strong FMR1 reactivation in response to demethylation (FIG. 3G), in agreement with prior observations (Liu et al., 2018). Surprisingly, however, we also observed a robust contraction of CGG repeats that was not previously reported (FIGS. 3H,3I). Further to our surprise, the control cell line harboring dCas9-Tet1-DEAD—which tethers a catalytically dead Tet1 (Tet1-DEAD) to dCas9 (Liu et al., 2018)—also exhibited significant CGG repeat contraction (FIGS. 3H,3I) and reactivation of FMR1 (FIG. 3G, p<0.0001 in 27 days). The degree of repeat contraction and FMR1 reactivation was smaller than for dCas9-Tet1 but was significantly above background. This was still perplexing given the inactive Tet1-DEAD. This finding is the first that suggested that CGG contraction could take place without DNA demethylation as a preceding event.

Although dCas9-Tet1-DEAD does not have a demethylase activity, the complex is targeted to FMR1 via the CGG gRNA (SEQ ID NO:1). We designed experiments to test the possibility that dCas9 would form a three-stranded nucleic acid structure known as an “R-loop” at the target site. R-loops comprise an annealed DNA:RNA hybrid and an associated unannealed single-stranded DNA. Previous studies using structural and single DNA molecule twitching analyses showed that CRISPR-Cas9 induces R-loop formation when bound to its target DNA (Szczelkun et al., 2014; Jiang et al., 2016; Jiang and Doudna, 2017). Catalytically dead Cas9 is also capable of forming R-loops (Szczelkun et al., 2014). When not properly cleared or shielded, R-loops can cause DNA breaks and genomic instability, resulting in the recruitment of the DNA repair machinery (Hegazy et al., 2020; Liu et al., 2020; Niehrs and Luke, 2020; Rinaldi et al., 2020). Notably, previous studies also suggested R-loops can form in the FMR1 locus of normal, PM, and FM FXS cells (Colak et al., 2014; Loomis et al., 2014).

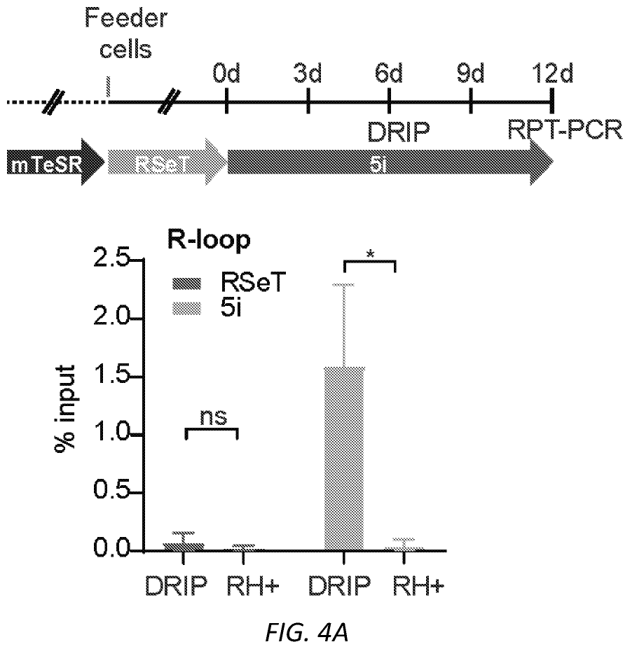

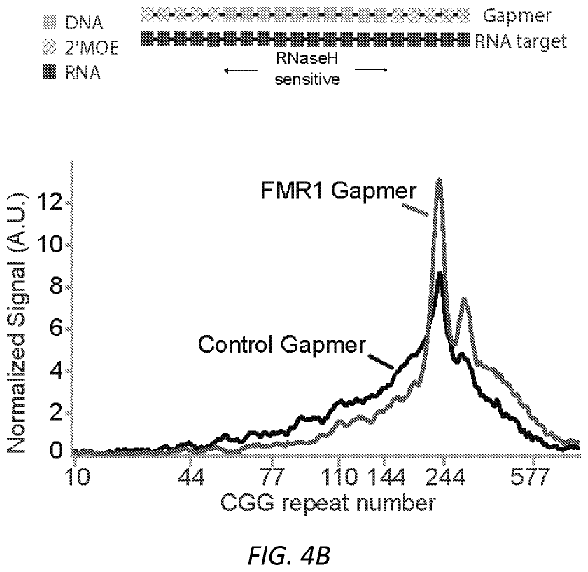

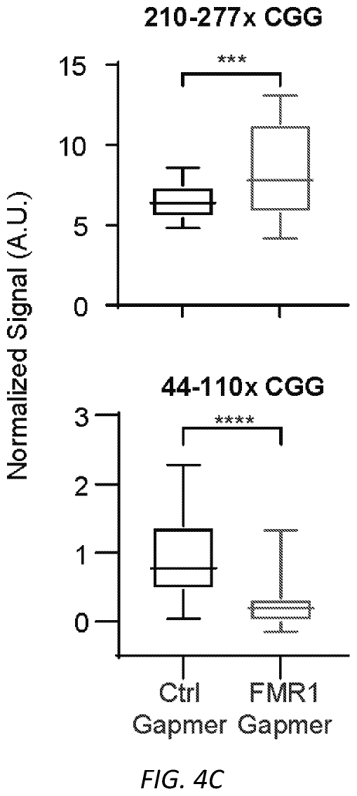

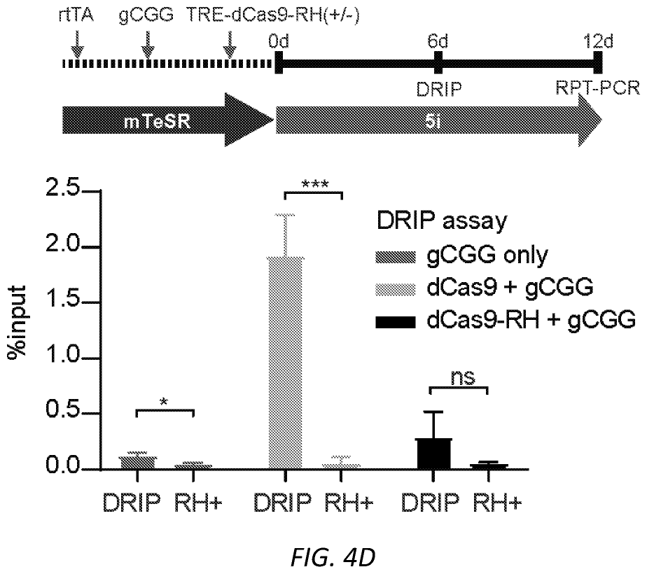

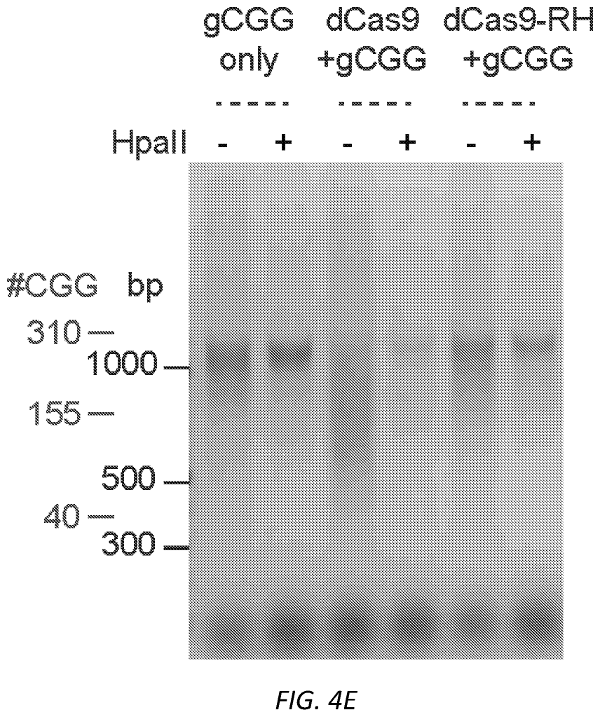

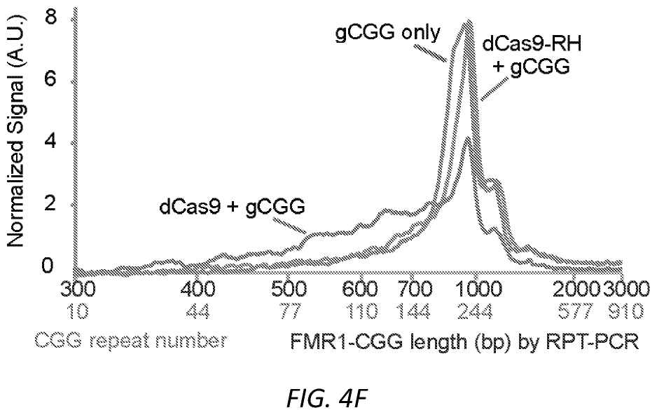

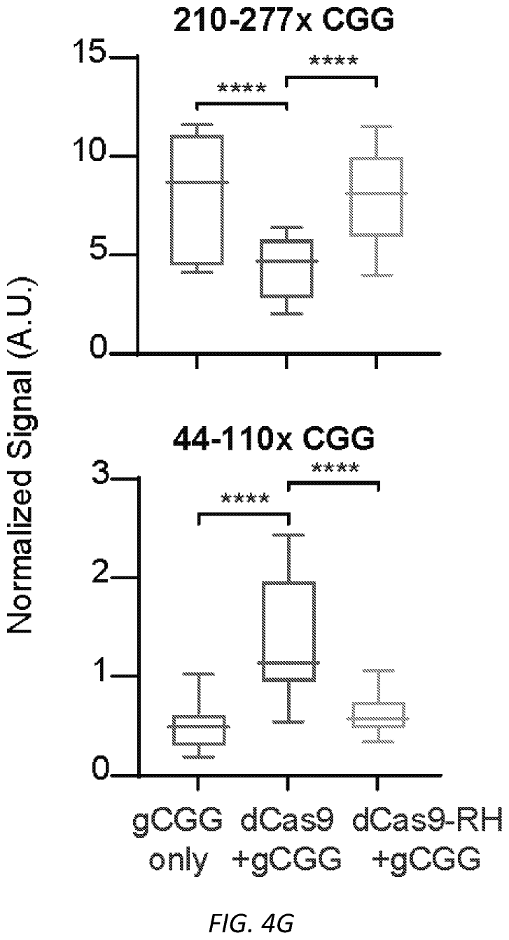

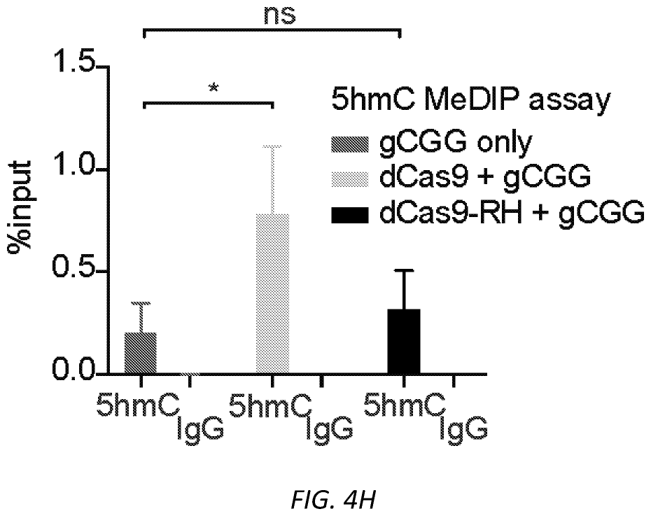

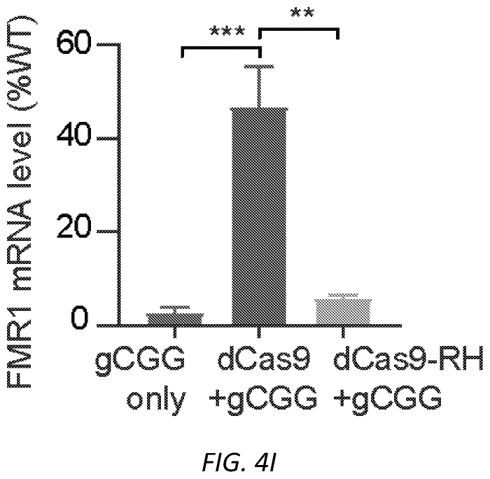

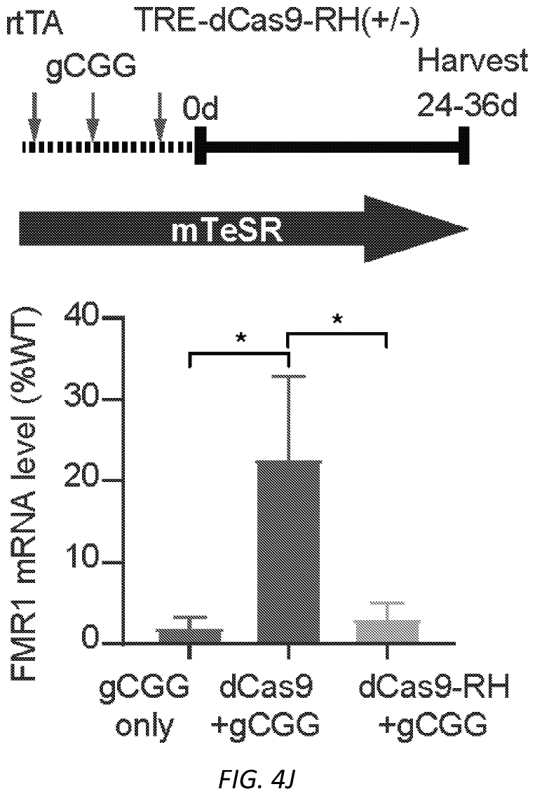

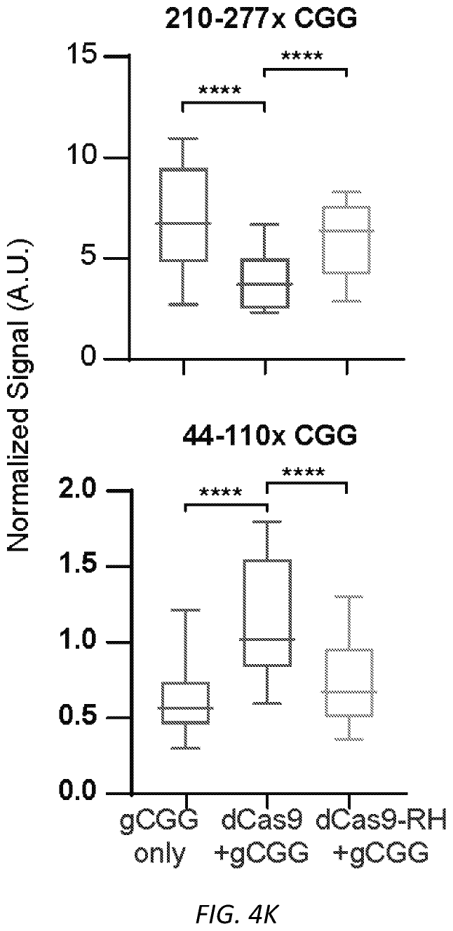

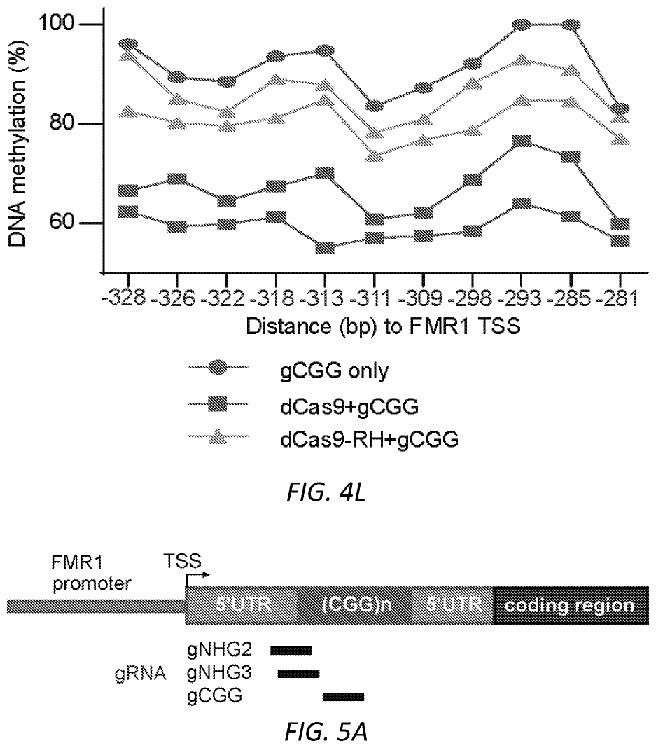

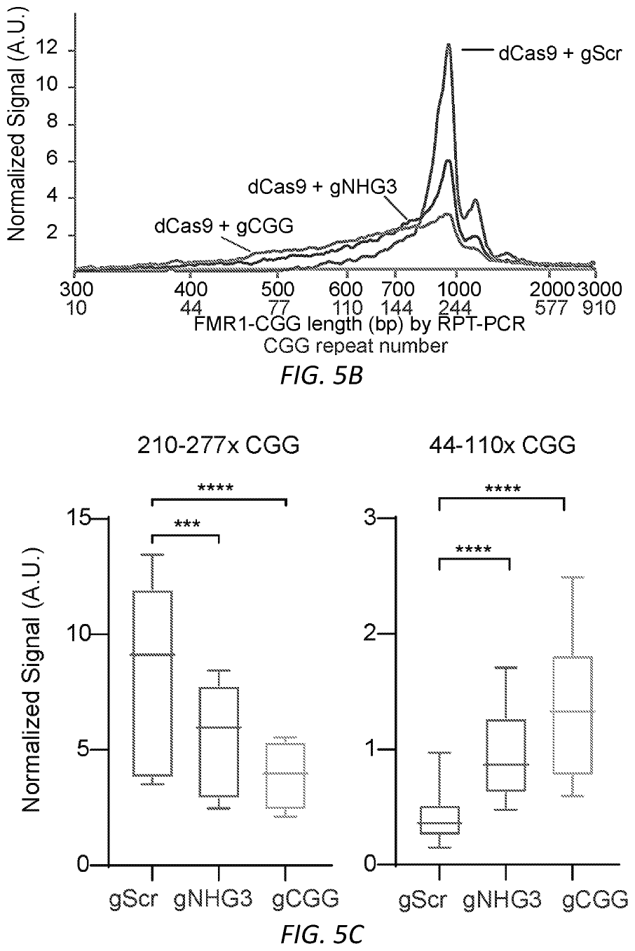

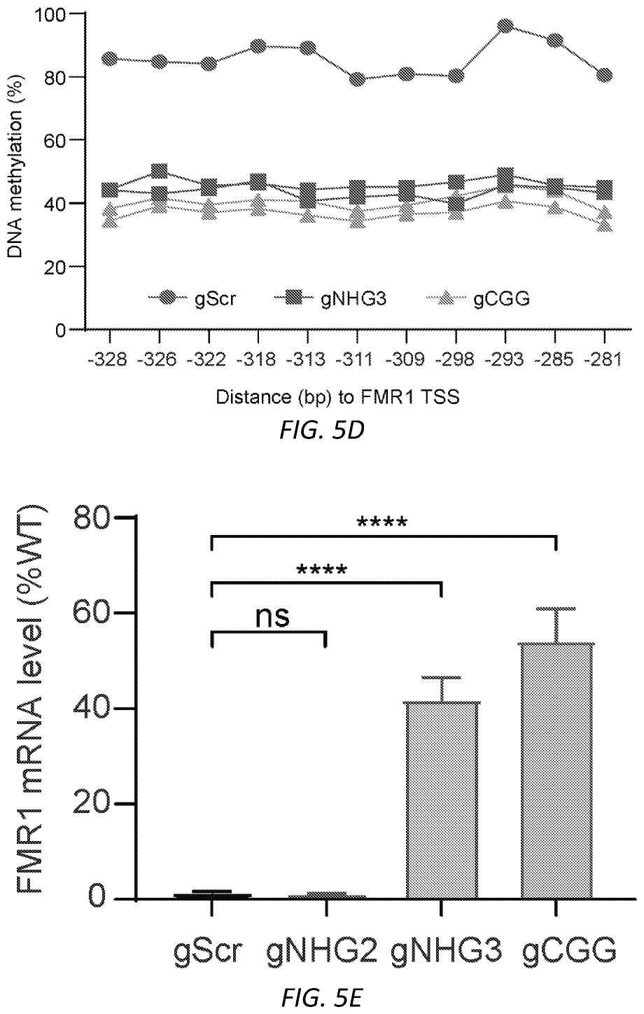

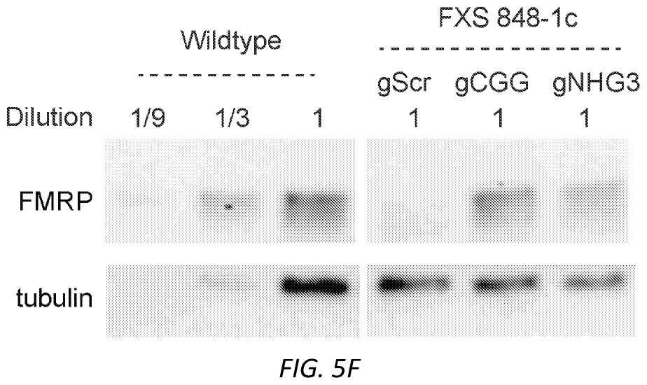

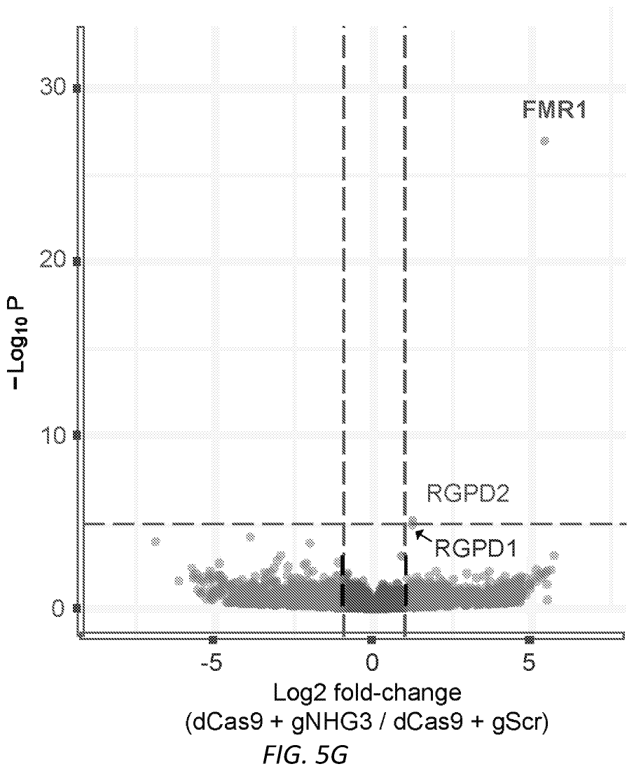

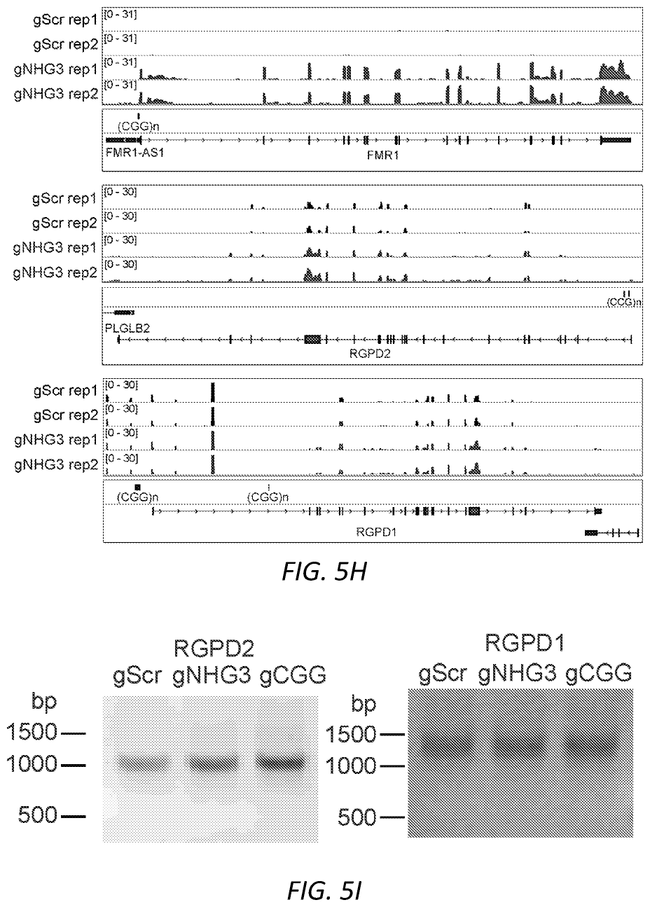

Therefore, we examined if R-loops were induced by conditions leading to CGG contraction. To monitor R-loop formation, we performed DRIP (DNA-RNA hybrid immunoprecipitation) using the established S9.6 antibody that specifically recognizes RNA-DNA hybrids (Phillips et al., 2013). We found a significant enrichment of RNA-DNA hybrids was evident at FMR1 in 848-1c cells grown in 5i media for 6 days (FIG. 4A). Pre-treatment with RNaseH (RH+) abolished DRIP signals, indicating a specific detection of R-loops. To test if the R-loops directed CGG repeat contraction, we targeted the CGG-containing nascent FMR1 mRNA with a gapmer to degrade the transcript and compared results obtained using a negative control containing all-2′MOE antisense oligonucleotide (ASO). Gapmer ASOs contain an internal stretch of unmodified DNA nucleotides which, when base-paired with RNA, degrade the RNA strand of RNA-DNA hybrid by recruiting endogenous RNaseH (Bennett and Swayze, 2010; Deleavey and Damha, 2012) (FIG. 4B, top), thereby destroying the R-loop. Using RPT-PCR, we observed an attenuation of CGG repeat contraction after FMR1 gapmer ASO treatment in comparison to scrambled gapmer ASO (Ctrl ASO) treatment (FIG. 11A). Bioanalyzer quantitation verified the loss of CGG contraction following FMR1 gapmer treatment (FIG. 5B,5C).

The sequences FMR1 gapmer ASO and the scrambled gapmer ASO used are as follows:

Gapmer-FMR1
(SEQ ID NO: 80)
/52MOErC/*/i2MOErC/*/i2MOErG/*/i2MOErC/*/i2MOErC/
*G*C*C*G*C*G*C*T*G*C*/i2MOErC/*/i2MOErG/*/
i2MOErC/*/i2MOErA/*/32MOErC/
Gapmer-Scramble
(SEQ ID NO: 81)
/52MOErG/*/i2MOErC/*/i2MOErG/*/i2MOErA/*/i2MOErC/
*T*A*T*A*C*G*C*G*C*A*/i2MOErA/*/i2MOErU/*/
i2MOErA/*/i2MOErU/*/32MOErG/

The * indicates a phosphorothioate bond, /52MOE indicates 5′end 2′MOE modified nucleotide, /32MOE indicates 3′end 2′MOE, and /12MOE indicates internal 2′MOE.

To further test the role of R-loops more directly, we introduced dCas9 without tethering Tet1 or Tet1-DEAD into 848-1c cells together with a CGG gRNA (gCGG). We transferred cells from mTeSR media to 5i media to determine whether dCas9+gCGG could accelerate the contraction associated with 5i treatment (FIG. 4D, top—timeline). DRIP analysis after 6 days of treatment confirmed much stronger enrichment of R-loops by dCas9 and gCGG in comparison to controls (FIG. 4D). Significantly, by 12 days, dCas9+gCGG together induced much stronger CGG contraction at FMR1 compared to the control without dCas9 (gCGG only) (FIGS. 4E-4G). The HpaII assay revealed an associated stronger demethylation of the FMR1 promoter region in dCas9+gCGG cells (FIG. 4E). Active demethylation by endogenous Tet proteins in an R-loop dependent way was suggested by a significant increase of the 5 hmC intermediate (FIG. 4H) (Arab et al., 2019). These effects were abolished by tethering RNaseH to dCas9 (dCas9−RH+gCGG), further demonstrating the dependence on formation of the RNA-DNA hybrid.

Significantly, treating for 6 days with dCas9+gCGG resulted in strong FMR1 gene reactivation to ˜50% of WT levels (FIG. 4I), suggesting much stronger reactivation relative to 5i alone (gCGG only). This accelerated reactivation was abolished when RNaseH was tethered to dCas9. Thus, without being bound by theory, under 5i conditions, R-loop formation at FMR1-CGG is an important feature driving the trinucleotide repeat contraction.

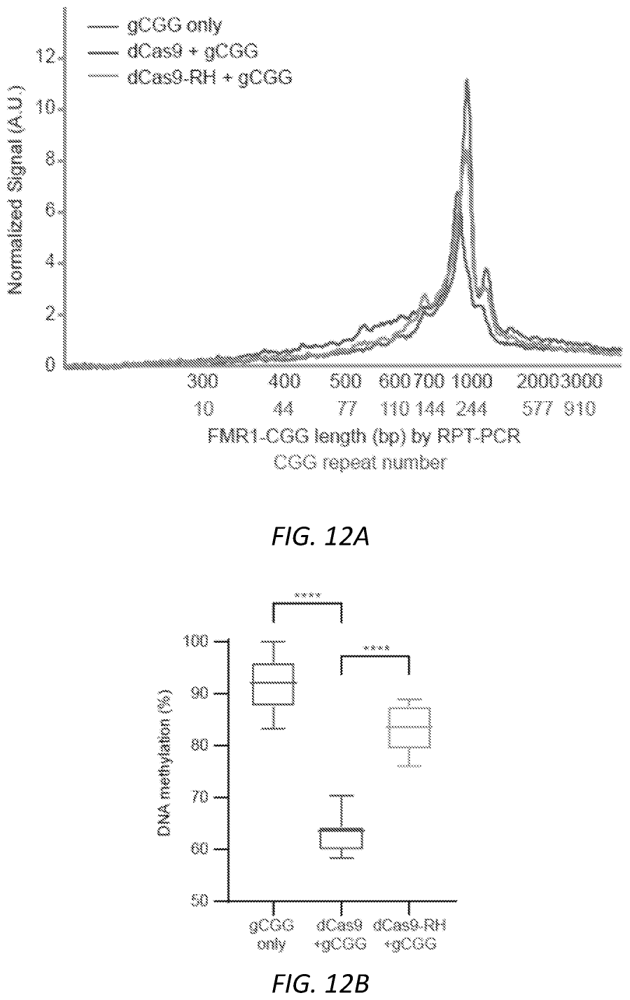

Because dCas9 by itself forms site-specific R-loops when targeted by specific gRNAs, we tested whether 5i was necessary at all. We repeated the experiment in mTeSR media without 5i (FIG. 4J). Intriguingly, dCas9+gCGG yielded 20-25% FMR1 reactivation after 24 days. This strong reactivation was also accompanied by CGG contraction (FIGS. 4K, 12A) and significant demethylation of the FMR1 promoter (FIGS. 4L, S12B). Tethering RNaseH to dCas9 significantly reduced contraction, demethylation, and FMR1 reactivation (FIGS. 4J-4L). We conclude that R-loop formation is both necessary and sufficient for CGG repeat contraction and FMR1 restoration in FXS.

### Example 6: On-Target Effects

Because CGG repeats appear in many other regions in the human genome, we designed FMR1-specific gRNAs to target dCas9 specifically to FMR1 by including unique sequences flanking the repeat (FIG. 5A). Using gNHG3, 848-1c FXS cells grown in mTeSR demonstrated effective CGG contraction (FIGS. 5B,5C), whereas gNHG2 did not. Importantly, gNHG3 showed a level of DNA demethylation and FMR1 reactivation that was as robust as seen with gCGG (FIGS. 5D-5F). Reactivation occurred at both the mRNA (FIG. 5E) and protein (FIG. 5F) levels.

The sequences used in these experiments are as follows:

FMR1-CGG-HG2 (gNHG2):
(SEQ ID NO: 8)
GGCGTGCGGCAGCGCGGCGG
FMR1-CGG-HG3 (gNHG3):
(SEQ ID NO: 9)
GTGCGGCAGCGCGGCGGCGG
NegativeControl-gRNA:
(SEQ ID NO: 11)
GGCACTGCGGCTGGAGGTGG

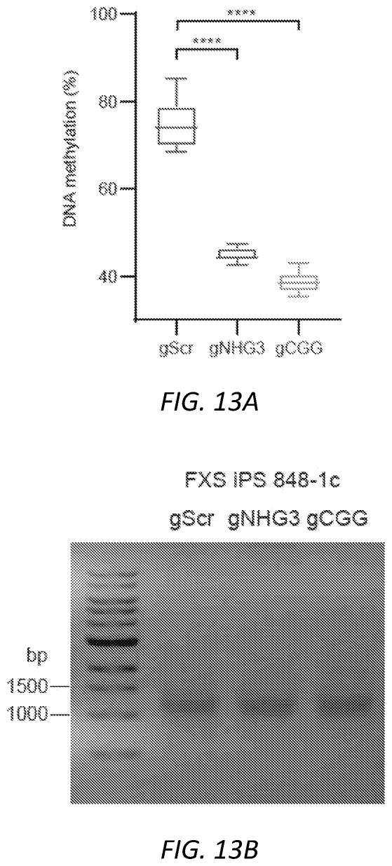

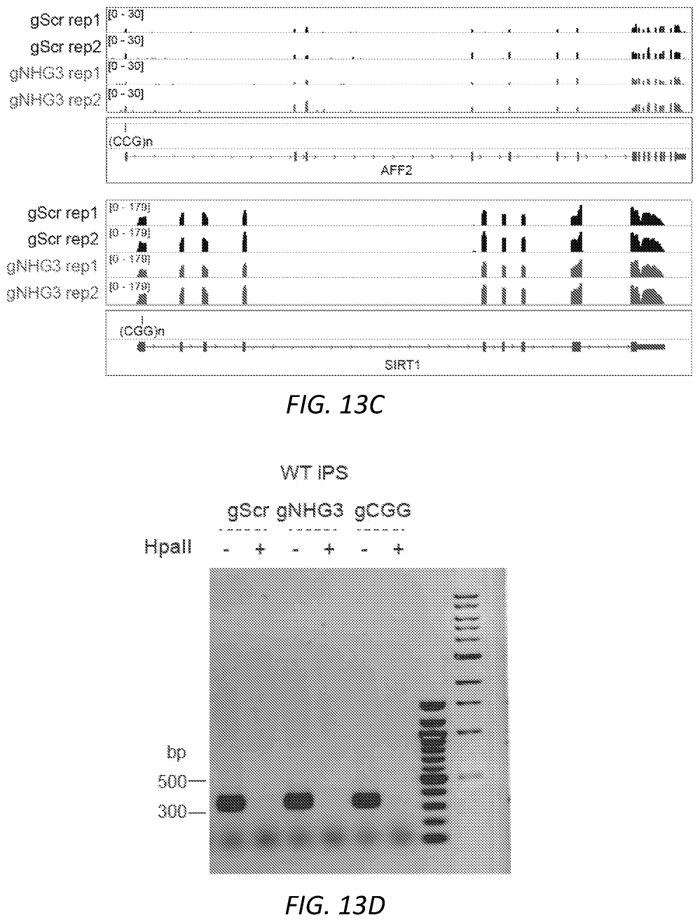

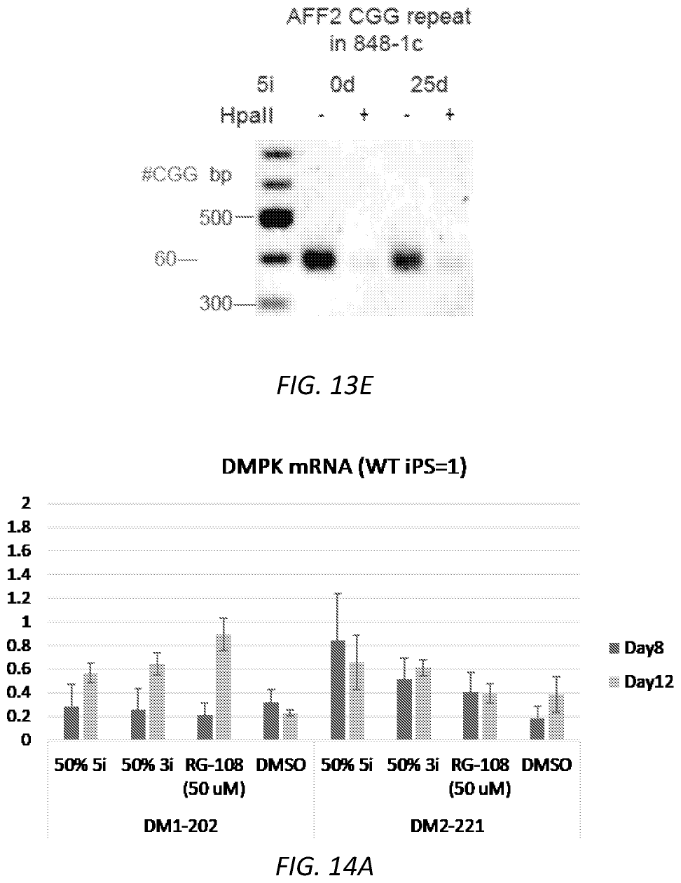

Given that gNHG3 is FMR1-specific, we examined on- versus off-target gene expression changes in this cell line after exposure to dCas9+gNHG3 for 36 days. Intriguingly, relative to a gScr control, differential gene expression analysis of two biological replicates showed only one dominant change—FMR1 (FIGS. 5G,5H). FMR1 was upregulated >40-fold compared to untreated cells. Only one other gene showed a significant, but much smaller change in expression: RGPD2 was upregulated by ˜2-fold (FIGS. 5G,5H). Closer examination of RGPD2 indicated that the locus contains two relatively long CGG repeat tracts of 174 bp and 129 bp separated by a spacer of 621 bp at its 5′ end (FIG. 5H), suggesting that it could have been an inefficient target of dCas9+gNHG3. However, RPT-PCR analysis did not reveal any obvious change in its CGG length after treatment (FIG. 5I). Interestingly, a related gene, RGPD1, also showed a minor change in expression, but the significance was below the threshold (FIG. 5H,5I). Notably, RGPD1 carries 6 short stretches of CGG repeats (177, 75, 191, 174, 202, and 55 bp) in its promoter region. This gene also showed no evident contraction after dCas9+gNHG3 treatment (FIGS. 5H, 5I, 13B). Other genes with CGG repeats around their 5′UTRs, including AFF2 and SIRT1, also showed no significant detectable gene expression level changes after treatment with dCas9+gNHG3 in the FXS cells (FIG. 13C). To determine whether normal cells respond to the treatment, we examined effects in wildtype hiPSCs expressing dCas9+gCGG or, alternatively, dCas9+gNHG3. No CGG contraction was observed at the normal CGG track of FMR1 (FIG. 13D). Altogether, our data indicate that dCas9+gNHG3 elicit strong site-specific effects at FMR1 with regards to CGG contraction and gene reactivation.

### Example 7: R-Loop Recruits the DNA Mismatch Repair Mechanism to Correct the CGG Repeat Expansion

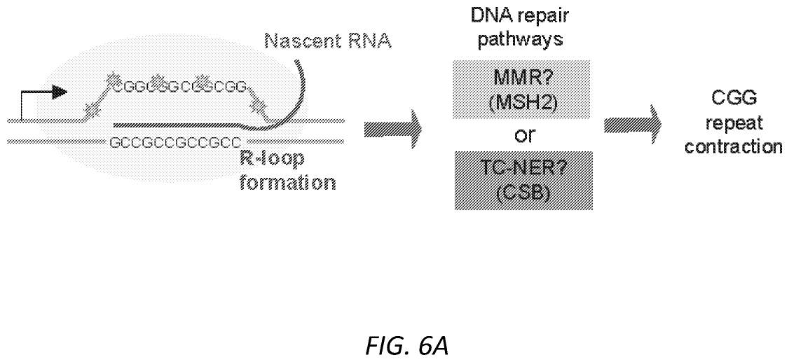

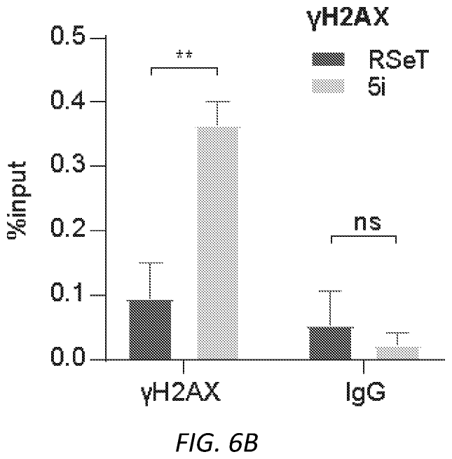

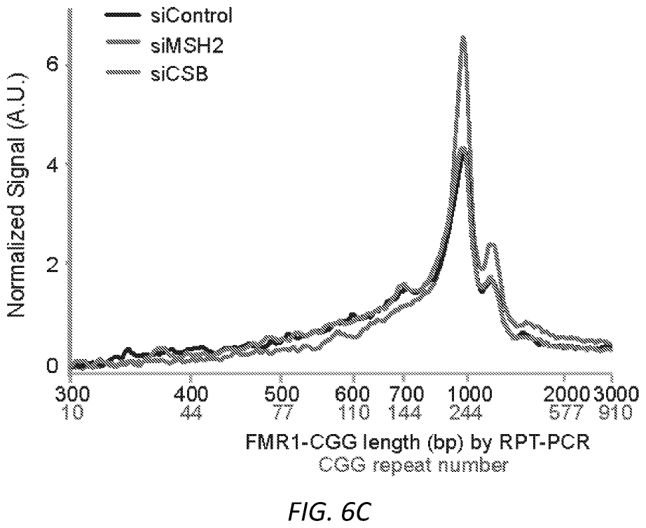

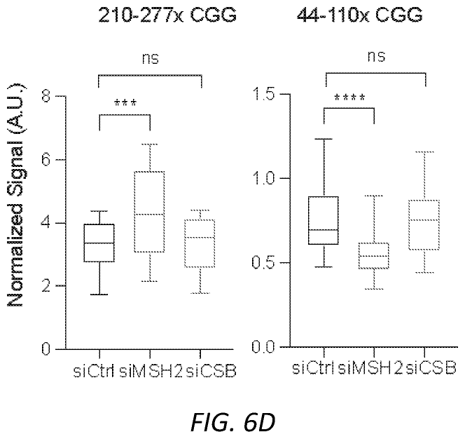

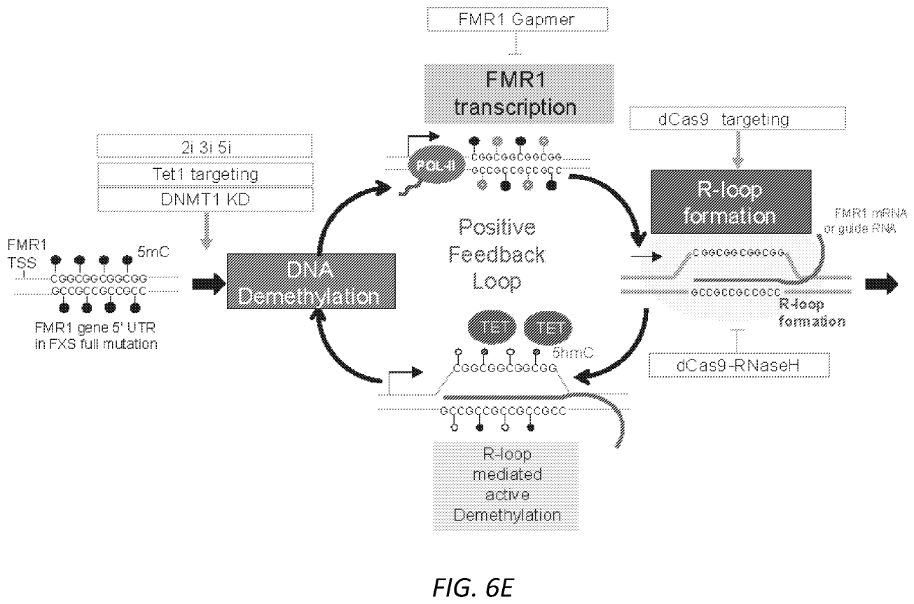

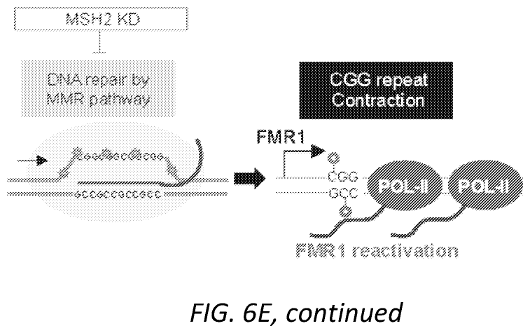

When not properly regulated, R-loops can cause DNA damage and trigger the DNA repair machinery (Rinaldi et al., 2020) and induce repeat instability (Lin et al., 2010; Freudenreich, 2018) (FIGS. 6A,6E). CGG copy number fluidity is known to depend on endogenous DNA repair mechanisms (Moore et al., 1999; Mirkin, 2007; Usdin et al., 2015). Several DNA repair pathways exist in mammalian cells to correct R-loop-mediated DNA damage (FIG. 6A). One pathway involves transcription-coupled nucleotide excision repair (TC-NER), which requires the core repair factor, CSB. Studies have shown that TC-NER plays a critical role in R-loop-induced DNA damage repair (Sollier et al., 2014) and trinucleotide repeat instability (Lin and Wilson (2007) Transcription-induced CAG repeat contraction in human cells is mediated in part by transcription-coupled nucleotide excision repair. Mol Cell Biol 27, 6209-6217; Lin and Wilson (2012) Nucleotide excision repair, mismatch repair, and R-loops modulate convergent transcription-induced cell death and repeat instability. PLOS One 7, e46807). The mismatch repair (MMR) pathway has also been shown to be involved in trinucleotide repeat instability. In this pathway, the MMR recognition factor, MSH2, binds to slipped-strand DNA structures formed within trinucleotide repeat tracts (Pearson et al., 1997; Owen et al., 2005) and causes repeat expansion and contraction in myotonic dystrophy, Huntington's disease, and other trinucleotide repeat disorders (Lin and Wilson, 2009; Lin et al., 2010; Nakatani et al., 2015).

We designed experiments to test if the observed CGG contraction induced by 5i invokes either DNA damage response. ChIP analysis showed that γ-H2AX, a histone mark for DNA damage response, was significantly elevated in 5i-treated cells at FMR1 locus (FIG. 6B), which supported the idea that aberrant R-loop formation at FMR1 creates DNA damage. We then tested if the damage is repaired by either the TC-NER/CSB or MMR/MSH2 pathway. Depleting CSB did not block CGG contraction in 5i-treated cells after 12 days, as the degree of contraction was as robust as that of control cells (FIGS. 6C-6D). By contrast, depleting MSH2 significantly attenuated CGG contraction (FIGS. 6C-6D). These data indicate that site-specific R-loop formation attracts the MMR pathway to correct the aberrant CGG repeat expansion at FMR1. Without being bound by theory, we propose that a positive feedback cycle of DNA demethylation, de novo FMR1 transcription, R-loop formation, and DNA mismatch repair leads to progressive trinucleotide repeat contraction and restorative FMR1 expression, as depicted in FIG. 6E.

### Example 8: Discussion

In this application, amongst other things, we have identified methods of FMR1 gene editing without use of exogenous nucleases such as TALENS, zing-finger nucleases, and CRISPR-Cas9. We initially observed that MEK and BRAF inhibitors together elicit CGG repeat contraction and full gene reactivation in <12 days. While MEK and BRAF kinases belong to the same MAPK pathway, inhibiting either kinase alone could not elicit the response, which suggested that non-overlapping downstream targets of MEKi and BRAFi are likely to mediate the effect. We found FMR1 promoter de-methylation occurs contemporaneously with repeat contraction and gene reactivation. Perturbation analysis showed that DNA demethylation plays an important role in unlocking FMR1 silencing but is not sufficient in itself to produce the full effect. We then traced the mechanism to formation of site-specific R-loops in the FMR1 CGG repeat and attraction of the MSH2-dependent mismatch repair pathway. Interestingly, R-loops created by targeting dCas9 to FMR1 were sufficient to induce re-expression of FMRP, and disrupting R-loops by degrading target strand RNA abolished CGG contraction and gene reactivation. Thus, without being bound by theory, we conclude that R-loops are both necessary and sufficient to achieve reactivation. Reactivation results not only in the induction of FMR1 mRNA but also production of FMRP to normal or near-normal levels.

Without being bound by theory, we propose that a positive feedback cycle of DNA demethylation, de novo FMR1 transcription, R-loop formation, and DNA mismatch repair leads to progressive trinucleotide repeat contraction and restorative FMR1 expression (FIG. 6E). A number of chromatin changes can initiate the positive cycle. A strong DNA demethylation, mediated either directly by DNMTi and/or indirectly by 2i, 3i, 5i, would open up FMR1 chromatin. Naive state media containing MEKi and BRAFi are also known to demethylate the hiPSC genome. Alternatively, unwinding of DNA by targeted dCas9 could also open up the FMR1 chromatin. The open chromatin conformation could then trigger de novo low-level FMR1 transcription. The initial transcription need not be robust. A low-level transcription would facilitate initial formation of R-loops, which would be stabilized at the FXS locus by the high GC content and sheer length of the CGG repeat. The aberrantly stable R-loops would then attract the DNA mismatch repair mechanism in an MSH2-dependent manner—through recognition of slipped-strand structures (Pearson et al., 1997; Owen et al., 2005)—to correct the structural defect in DNA, resulting in a round of CGG excision/repair. R-loops are also known to recruit endogenous TET1 demethylase and facilitate CpG demethylation (Arab et al., 2019) and protect underlying DNA loci from de novo DNA methylation (Ginno et al., 2012). Indeed, we observed increased 5 hmC levels (a chromatin mark of TET1 action) during CGG contraction (FIGS. 4H,11C). Thus, we envisage that this sequence of events would repeat itself in a positive feedback loop and lead to progressive demethylation, nascent FMR1 transcription, MMR recruitment, CGG excision, and FMR1 reactivation as depicted in FIG. 6E.

To the limits of our detection, the method is surprisingly site-specific (FIGS. 5A-5I). In FXS cells treated with dCas9+gNHG3, transcriptomic analysis indicates a single major target, FMR1, which was upregulated >30-fold compared to control-treated cells. Other CGG repeat-containing genes (e.g., RGPD1, SIRT1, AFF2) were not affected significantly, except for RGPD2, which contains two CGG tracts of 174 and 129 bp and was upregulated by ˜2-fold. However, while FMR1 repeats were contracted, RGPD2 repeats were not evidently so. We therefore believe that CGG repeat length is a key determinant of whether a gene is sensitive to R-loops. For instance, wildtype cells with <50 CGG repeats and FXS pre-mutation cells with ˜150×CGG do not initiate contraction (FIGS. 8A-8B,13E). Moreover, in FXS cells, genes with <50 CGG repeats also do not appear to be susceptible to contraction (FIGS. 7I,13D). Thus, there may be a copy number threshold below which contraction cannot initiate. However, while contraction does not initiate under these conditions, cells with the full mutation (>200 CGG repeats) can clearly contract below that threshold. For example, once induced to initiate contraction, cell line 848-1c with its ˜300 CGG repeats can continue to contract below 150 CGG repeats (FIGS. 1D,2A,5E). The reasons for a contraction threshold are currently unknown, but it is known that longer CGG repeats have the potential to form complex secondary structures in both the RNA and DNA (Gacy et al., 1995; Pearson and Sinden, 1996; Mirkin, 2007), and secondary structures formed in the non-template DNA strand could aid in R-loop stability and recruitment of additional mediators (Hirst and White, 1998). In summary, our results demonstrated that R-loop mechanisms can achieve up to 40-100% FMR1 reactivation. If FXS cellular phenotypes could be reversed with FMR1 restoration (Hagerman et al., 2014; Berry-Kravis et al., 2018; Hagerman and Hagerman, 2021), an approach to treating FXS in the future could involve targeting R-loops for trinucleotide repeat contraction and re-expression of the missing FMRP in neuronal cells.

### Example 9: DMPK and SIX5 Reactivation in DM1 Cells

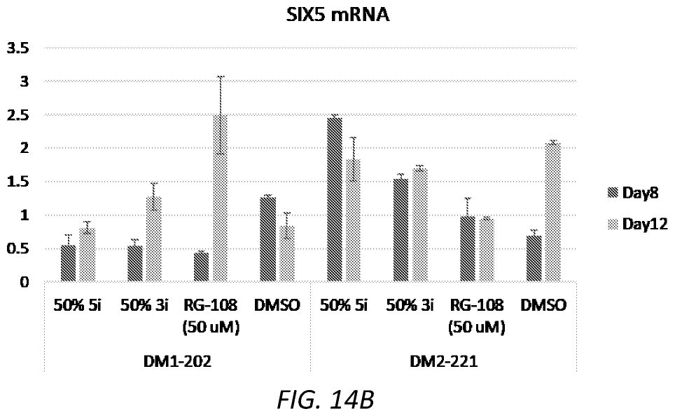

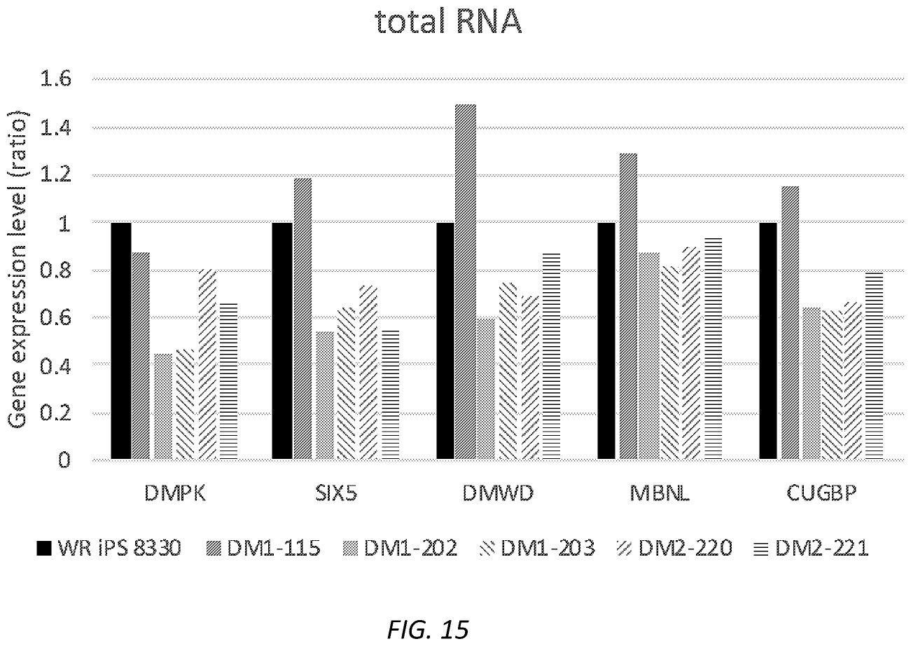

Treatment using 50:50 5i and RSET, or 50:50 3i and RSET, and even RG108 alone, gave good reactivation of the two genes flanking the DM1 CTG repeat (DMPK and SIX5; FIGS. 14A-15). Dystrophia myotonica protein kinase (DMPK) and SIX homeobox 5 (SIX5) mRNA levels were analyzed by RT-qPCR. The iPSC isolated from DM1 patient, DM1-115, and one from DM2 patient, DM2-221, were treated with 5i (PD0325901:0.5 uM, Rocki:0.75 uM, IM-12:0.5 uM, SB590885: 0.25 uM, WH4-023:05 uM), 3i (PD0325901:0.5 uM, Rocki:0.75 uM, SB590885: 0.25 uM), RG-108 (50 uM), and DMSO control for 8 days and 12 days respectively. Total RNA was extracted by Trizol reagent (Thermofisher) following the manufacture's protocol. To remove the residual genomic DNA, DNaseI (TURBO DNase, Thermofisher) was done before reverse transcription with Super ScriptIII reverse transcriptase (Thermofisher). GAPDH was used as internal control for normalization. Gene expression shown as relative level normalized with DMSO treated wild type iPSC. The primers used are as follows:

DMPK-qPCR-Ori-Fwd
(SEQ ID NO: 82)
CACCGACACATGCAACTTCGAC
DMPK-qPCR-Ori-Rev
(SEQ ID NO: 83)
AGTAGCCCACAAAAGGCAGGTG
SIX5-qPCR-Ori-Fwd
(SEQ ID NO: 84)
TCACGCAGGTCAGCAACTGGTT
GAPDH-Fwd
(SEQ ID NO: 85)
GAAGGTGAAGGTCGGAGTC
GAPDH-Rev
(SEQ ID NO: 86)
GAA GATGGTGATGGGATTTC

There was an observable reactivation in DM1 cells as both DMPK mRNA and SIX5 mRNA increased, particularly following 12 days of treatment (FIGS. 14A and 14B, respectively). The DM2 cells did not respond as strongly because DM2 is not linked to SIX5 or DMPK, and thus served as a good control.

### Example 10. Repeat Contraction in PD Cells Using dCas9+gRNA Treatment and Using Small Molecule Treatment

Experiments are conducted in PD cells to test the effect of the dCas9+gRNA on retracting nucleotide repeats. The PD cells are treated with dCas9+gRNA for 6, 12, and/or 18 days, after which the cells are assayed via RPT-PCR. The Purified RPT-PCR products are further analyzed by Bioanalyzer to monitor the changes in the length of GGC repeat fragments. The data is also analyzed to determine whether the dCas9+gRNA reactivate the NOTCH2NLC gene.

Experiments are conducted in PD cells to test the effect of the small molecule cocktails on retracting trinucleotide repeats. The PD cells are treated with combinations of small molecules for 6, 12, and/or 18 days, after which the cells are assayed via RPT-PCR. The Purified RPT-PCR products are further analyzed by Bioanalyzer to monitor the changes in the length of GGC repeat fragments. The data is analyzed to determine whether the (P+S) and (P+S+R) combinations reactivate the NOTCH2NLC gene.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of contracting an expansion of nucleotide repeats in a gene in a cell, the method comprising contacting the cell with or expressing in the cell an inactive Cas9 protein and a guide RNA (gRNA) that directs the Cas9 to the gene and/or the nucleotide repeat, in an amount sufficient to reduce the number of nucleotide repeats in the cell, and optionally contacting the cell with or expressing in the cell a DNMT inhibitor.

2. The method of claim 1, wherein the expansion is in a fragile X mental retardation 1 (FMR1) gene, wherein the FMR1 gene is inactive due to the presence of expansion of CGG nucleotide repeats in the 5′-UTR of the FMR1 gene, and wherein the number of nucleotide repeats in at least one allele of the gene is reduced after contact with the dCas9 and gRNA, e.g., wherein the number of CGG nucleotide repeats in the 5′-UTR of at least one allele of the FMR1 gene is reduced after contact with the dCas9 and gRNA.

3. The method of claim 1 or 2, wherein the cell is from a subject who has a disorder caused by the expansion of nucleotide repeats, e.g., a neurodevelopmental or neurodegenerative disorder caused by the expansion of CGG nucleotide repeats in the 5′-UTR of the FMR1 gene.

4. The method of claim 3, wherein the cell is an induced pluripotent stem cell (iPSC) derived from a somatic cell of a subject who has a disorder caused by the expansion of nucleotide repeats, e.g., a subject who has Fragile X syndrome (FXS).

5. The method of claim 1 or 2, wherein the cell is from a subject who has >200 CGG repeats, or 50-200 CGG repeats.

6. A population of cells generated by the methods of claims 1-5.

7. A method of treating a subject who has a condition associated with nucleotide repeat expansion in a gene, the method comprising:
obtaining iPSC derived from differentiated somatic cells obtained from the subject;
exposing the iPSC to, or expressing in the iPSC, an inactive Cas9 protein and a guide RNA (gRNA) that directs the dCas9 to the gene and/or the nucleotide repeat, in an amount sufficient to reduce the number of nucleotide repeats in the cell for a time and under conditions sufficient for reduction of the number of nucleotide repeats;
optionally, exposing the iPSC to, or expressing in the iPSC, a DNMT inhibitor;
promoting differentiation of the reactivated cells to neural precursor cells or other neuronal cell types; and administering the cells to the brain or spinal cord of the subject with a condition associated with nucleotide repeat expansion.

8. A method of treating a subject who has a condition associated with nucleotide repeat expansion in a gene, the method comprising administering to the subject a therapeutically effective amount of an inactive Cas9 protein and a guide RNA that directs the dCas9 to the gene and/or the nucleotide repeat, in an amount sufficient to reduce the number of nucleotide repeats in the cell, and optionally, administering to the subject a therapeutically effective amount of a DNMT inhibitor.

9. A method of contracting nucleotide repeats in a gene a living cell, e.g., a cell having a number of repeats above a reference number or in a reference range, the method comprising contacting the cell with an inactive Cas9 protein and a guide RNA that directs the dCas9 to the gene and/or the nucleotide repeat, in an amount sufficient to reduce the number of nucleotide repeats in the cell, and optionally, contacting the cell with a DNMT inhibitor.

10. The method of claim 9, wherein the reference number is 20, 21, 22, 23, 24, 25, 26, 27, 28, 29, 30, 31, 32, 33, 34, 35, 36, 37, 38, 39, 40, 45, 50, 55, 60, 75, 100, or 200 repeats; or the range is 20-40 repeats, 30-100 repeats, 40-200 repeats, or 50-200 repeats.

11. The method of claim 9, wherein the reference number is the number of nucleotide repeats in a healthy cell not having a condition associated with nucleotide repeat expansion or a control cell.

12. The method of claim 9, wherein the reference number is 0-30 repeats, 0-40, or 0-50 repeats.

13. The method of claim 9, wherein the nucleotide repeats comprise any one or more of CGG repeats, GGC repeats, CAG repeats, CTG repeats, GAA repeats, CCCCGG repeats, CCTG repeats, ATTCT repeats, CAAA repeats, and TGGAA repeats.

14. The method of claims 9-12, wherein the cell is in a living subject.

15. The method of claim 13, wherein the cell is in the brain of the subject.

16. The method of claim 8 or 14, wherein the dCas9 and gRNA are administered to the CNS, or is administered systemically to the subject.

17. The method of claim 9, wherein the cell is an iPSC derived from differentiated somatic cell obtained from a subject who has a condition associated with nucleotide repeat expansion.

18. The method of any one of claim 7, 8, 11, or 17, wherein the condition associated with nucleotide repeat expansion is any one or more of FXS, FXTAS, PD, HD, or myotonic dystrophy, and other repeat disorders.

19. A method of preparing a population of neural precursor cells or other neuronal cell type with decreased nucleotide repeats, the method comprising
obtaining iPSC derived from differentiated somatic cells obtained from a subject who has a condition associated with nucleotide repeat expansion;
exposing the iPSC to an inactive Cas9 protein and a guide RNA that directs the dCas9 to the gene and/or the nucleotide repeat for a time and under conditions sufficient for reduction of the number of nucleotide repeats;
optionally, exposing the iPSC to a DNMT inhibitor; and
promoting differentiation of the iPSC to neural precursor cells or other neuronal cell type,
thereby promoting differentiation of the reactivated cells to neural precursor cells or other neuronal cell types.

20. A population of neural precursor cells or other neuronal cell type with decreased nucleotide repeats prepared using the method of claim 19.

21. A method of reactivating an inactive gene in a living cell, wherein the gene is inactive due to the presence of expansion of nucleotide repeats, optionally wherein the inactive gene is fragile X mental retardation 1 (FMR1) and the FMR1 gene is inactive due to the presence of expansion of nucleotide repeats (e.g., CGG nucleotide repeats in the 5′-UTR of the FMR1 gene), the method comprising contacting the cell with a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor; or
(v) a MEK inhibitor, a Raf inhibitor, a ROCK1 inhibitor, a GSK-3β inhibitor, and a Src Inhibitor, for a time and under conditions sufficient for reactivation of the gene, and optionally contacting the cell with or expressing in the cell a DNMT inhibitor.

22. The method of claim 21, wherein the number of nucleotide repeats in at least one allele of the gene is reduced after contact with the reactivation cocktail, e.g., wherein the number of CGG nucleotide repeats in the 5′-UTR of at least one allele of the FMR1 gene is reduced after contact with the reactivation cocktail.

23. The method of claim 21 or 22, wherein the cell is from a subject who has a disorder caused by the expansion of nucleotide repeats, e.g., a neurodevelopmental or neurodegenerative disorder, e.g., a disorder caused by the expansion of CGG nucleotide repeats in the 5′-UTR of the FMR1 gene.

24. The method of claim 23, wherein the cell is an induced pluripotent stem cell (iPSC) derived from a somatic cell of a subject who has a disorder caused by the expansion of nucleotide repeats, e.g., a subject who has Fragile X syndrome (FXS).

25. The method of claim 21 or 22, wherein the cell is from a subject who has >200 CGG nucleotide repeats, or 50-200 CGG repeats.

26. A population of cells generated by the methods of claims 21-25.

27. A method of treating a subject who has a condition associated with nucleotide repeat expansion in a gene, the method comprising:
obtaining iPSC derived from differentiated somatic cells obtained from the subject;
exposing the iPSC to a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor;
(v) a MEK inhibitor, a Raf inhibitor, a ROCK1 inhibitor, a GSK-3β inhibitor, and a Src Inhibitor, for a time and under conditions sufficient for reduction of the number of nucleotide repeats; optionally, exposing the iPSC to, or expressing in the iPSC, a DNMT inhibitor;
promoting differentiation of the reactivated cells to neural precursor cells or other neuronal cell types; and administering the cells to the brain or spinal cord of the subject with a condition associated with nucleotide repeat expansion.

28. A method of treating a subject who has a condition associated with nucleotide repeat expansion, the method comprising administering to the subject a therapeutically effective amount of a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor;
(v) a MEK inhibitor, a Raf inhibitor, a ROCK1 inhibitor, a GSK-3β inhibitor, and a Src Inhibitor, and optionally, administering to the subject a therapeutically effective amount of a DNMT inhibitor.

29. A method of contracting nucleotide repeats in a living cell, e.g., a cell having a number of repeats above a reference number or in a reference range, the method comprising contacting the cell with a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor; or
(v) a MEK inhibitor, a Raf inhibitor, a ROCK1 inhibitor, a GSK-3β inhibitor, and a Src Inhibitor, for a time and under conditions sufficient for contraction of the nucleotide repeats, and optionally, contacting the cell with a DNMT inhibitor.

30. The method of claim 29, wherein the reference number is 20, 21, 22, 23, 24, 25, 26, 27, 28, 29, 30, 31, 32, 33, 34, 35, 36, 37, 38, 39, 40, 45, 50, 55, 60, 75, 100, or 200 repeats; or the range is 20-40 repeats, 30-100 repeats, 40-200 repeats, or 50-200 repeats.

31. The method of claim 29, wherein the reference number is the number of nucleotide repeats in a healthy cell not having a condition associated with nucleotide repeat expansion or a control cell.

32. The method of claim 29, wherein the reference number is 0-30 repeats, 0-40, or 0-50 repeats.

33. The method of claim 29, wherein the nucleotide repeats comprise any one or more of CGG repeats, GGC repeats, CAG repeats, CTG repeats, GAA repeats, CCCCGG repeats, CCTG repeats, ATTCT repeats, CAAA repeats, and TGGAA repeats.

34. The method of claims 29-32, wherein the cell is in a living subject.

35. The method of claim 33, wherein the cell is in the brain of the subject.

36. The method of claim 28 or 34, wherein the reactivation cocktail is administered to the CNS or is administered systemically to the subject.

37. The method of claim 29, wherein the cell is an iPSC derived from differentiated somatic cell obtained from a subject who has a condition associated with nucleotide repeat expansion.

38. The method of any one of claim 27, 28, 31, or 37, wherein the condition associated with nucleotide repeat expansion is any one or more of FXS, FXTAS, PD, HD, or myotonic dystrophy.

39. A method of preparing a population of neural precursor cells or other neuronal cell type with decreased nucleotide repeats, the method comprising
obtaining iPSC derived from differentiated somatic cells obtained from a subject who has a condition associated with nucleotide repeat expansion;
exposing the iPSC to a reactivation cocktail with active factors comprising or consisting of:
(i) a MEK inhibitor and a Raf inhibitor;
(ii) a MEK inhibitor, a Raf inhibitor, and a ROCK1 inhibitor;
(iii) a MEK inhibitor, a Raf inhibitor, and a GSK-3β inhibitor;
(iv) a MEK inhibitor, a Raf inhibitor, a ROCK1 inhibitor, and a GSK-3β inhibitor;
(v) a MEK inhibitor, a Raf inhibitor, a ROCK1 inhibitor, a GSK-3β inhibitor, and a Src Inhibitor, for a time and under conditions sufficient for reduction of the number of nucleotide repeats,
optionally, exposing the iPSC to a DNMT inhibitor; and
promoting differentiation of the iPSC to neural precursor cells or other neuronal cell type, thereby promoting differentiation of the reactivated cells to neural precursor cells or other neuronal cell types.

40. A population of neural precursor cells or other neuronal cell type with decreased nucleotide repeats prepared using the method of claim 39.

41. The method of any one of claims 1, 7-9, 19, 21, 27-29, and 39, wherein the DNMT inhibitor is at least one of is RG108, 5-azacytidine, decitabine, Zebularine, procainamide, procaine, psammaplin A, sinefungin, temozolomide, OM173-alphaA, DNMT3A-binding protein, theaflavin 3,3′-digallate, 1-Hydrazinophthalazine, SGI-1027, hydralazine, NSC14778, Olsalazine, Nanaomycin, SID 49645275, Δ2-isoxazoline, epigallocatechin-3-gallate (EGCG), MG98, SGI-110, SGI-1027, SW155246, SW15524601, SW155246-2, or DZNep, an ASO targeting DNMT, optionally comprising SEQ ID NO: 87, TCAAGTTGAGGCCAGAAGGA, or an siRNA targeting DNMT, optionally comprising SEQ ID NOs: 72-75.
